SEQUENCE LISTING (73 sequences)

SEQ ID NO: 1 - DNA, 348 bp, Kosakonia sacchari
  source 1..348: mol_type = other DNA
cgtcctgtaataataaccggacaattcggactgattaaaaaagcgcccttgtggcgctttttttatattcccgcctccatttaaaataaaaaatccaatcggatttcactatttaaactggccattatctaagatgaatccgatggaagctcgctgttttaacacgcgttttttaaccttttattgaaagtcggtgcttctttgagcgaacgatcaaatttaagtggattcccatcaaaaaaatattctcaacctaaaaaagtttgtgtaatacttgtaacgctacatggagattaactcaatctagagggtattaataatgaatcgtactaaactggtactgggcgc

SEQ ID NO: 2 - DNA, 333 bp, Kosakonia sacchari
  source 1..333: mol_type = other DNA
tcaccacggcgataaccataggttttcggcgtggccacatccatggtgaatcccactttttccagcacgcgcgccacttcatcgggtcttaaatacatagattttcctcgtcatctttccaaagcctcgccaccttacatgactgagcatggaccgtgactcagaaaattccacaaacgaacctgaaaggcgtgattgccgtctggccttaaaaattatggtctaaactaaaatttacatcgaaaacgagggaggatcctatgtttaacaaaccgaatcgccgtgacgtagatgaaggtgttgaggatattaaccacgatgttaaccagctcg

SEQ ID NO: 3 - DNA, 315 bp, Kosakonia sacchari
  source 1..315: mol_type = other DNA
atcatattgcgctccctggttatcatttgttactaaatgaaatgttataatataacaattataaataccacatcgctttcaattcaccagccaaatgagaggagcgccgtctgacatagccagcgctataaaacatagcattatctatatgtttatgattaataactgatttttgcgttttggatttggctgtggcatccttgccgctcttttcgcagcgtctgcgtttttgccctccggtcagggcatttaagggtcagcaatgagtttttacgcaattacgattcttgccttcggcatgtcgatggatgcttt

SEQ ID NO: 4 - DNA, 339 bp, Kosakonia sacchari
  source 1..339: mol_type = other DNA
tgacgaggcaggttacatcactggtgaaaccctgcacgtcaatggcggaatgtatatggtttaaccacgatgaaaattatttgcgttattagggcgaaaggcctcaaaatagcgtaaaatcgtggtaagaactgccgggatttagttgcaaatttttcaacattttatacactacgaaaaccatcgcgaaagcgagttttgataggaaatttaagagtatgagcactatcgaagaacgcgttaagaaaattatcggcgaacagctgggcgttaagcaggaagaagttaccaacaatgcttccttcgttgaagacctgggcgctgattctcttgacaccg

SEQ ID NO: 5 - DNA, 313 bp, Kosakonia sacchari
  source 1..313: mol_type = other DNA
ggacatcatcgcgacaaacaatattaataccggcaaccacaccggcaatttacgagactgcgcaggcatcctttctcccgtcaatttctgtcaaataaagtaaaagaggcagtctacttgaattacccccggctggttgagcgtttgttgaaaaaaagtaactgaaaaatccgtagaatagcgccactctgatggttaattaacctattcaattaagaattatctggatgaatgtgccattaaatgcgcagcataatggtgcgttgtgcgggaaaactgcttttttttgaaagggttggtcagtagcggaaac

SEQ ID NO: 6 - DNA, 330 bp, Kosakonia sacchari
  source 1..330: mol_type = other DNA
ccatccggttaagcgtatcgaagaagttcttgcccttgcgctgcagaatgaaccctttggtatgcaagtcgtaacggcaaaatagtgaccttgcgcaaagtgcgttaataaaaacaaggttggtgagtgatttcggacttgccagcctttttttgtatagctaatttagattgctggttgggtgtgccatcatcaactggtgttgtaagggcatgacaggcctgatataactgctgcgcggtcgcgctgtgaaggattcaggtgcgatataaattataaagagaggaagagtagagtgaataaatctcaactggtagacaagattgccgc

SEQ ID NO: 7 - DNA, 339 bp, Kosakonia sacchari
  source 1..339: mol_type = other DNA
cgcgtcaggttgaacgtaaaaaagtcggtctgcgcaaagcacgtcgtcgtccgcagttctccaaacgttaattggtttctgcttcggcagaacgattggcgaaaaaacccggtgcgaaccgggtttttttatggataaagatcgtgttatccacagcaatccattgattatctcttctttttcagcatttccagaatcccctcaccacaaagcccgcaaaatctggtaaactatcatccaattttctgcccaaatggctgggattgttcattttttgtttgccttacaacgagagtgacagtacgcgcgggtagttaactcaacatctgaccggtcgat

SEQ ID NO: 8 - DNA, 345 bp, Kosakonia sacchari
  source 1..345: mol_type = other DNA
cagagccgggttgttgatccgcagggcgtgacggttgcggcagcagcagaagcgccacagctgattttcgcagaggtcacgcctgaacgcgtggcgcagacacgcgagaaactgccggtattacgcaatcgccgtttcgctgtaccgcatttattgtgatgtttttttaaacaatgcttgattcatctcgttacacattgctattgtgtgcgcgcgtcgaatgaccgttaatgaagtccggttataatggcgttttatgcagcctgttttaagaaagaaggtatctatgggtgagattagtattaccaaactgctggttgtggccgcactggttgttctgctgtt

SEQ ID NO: 9 - DNA, 350 bp, Kosakonia sacchari
  source 1..350: mol_type = other DNA
atattgacaccatgacgcgcgtaatgctgattggttctgtgacgctggtaatgattgtcgaaattctgaacagtgccatcgaagccgtagtagaccgtattggtgcagaattccatgaactttccgggcgggcgaaggatatggggtcggcggcggtgctgatgtccatcctgctggcgatgtttacctggatcgcattactctggtcacattttcgataacgcttccagaattcgataacgccctggttttttgcttaaatttggttccaaaatcgcctttagctgtatatactcacagcataactgtatatacacccagggggcgggatgaaagcattaacggccagg

SEQ ID NO: 10 - DNA, 297 bp, Kosakonia sacchari
  source 1..297: mol_type = other DNA
cctgtatgaagatggcgtgcgcaaagatcgcctggataacagcgatatgattagccagcttgaagcccgcattcgcgcgaaagcgtcaatgctggacgaagcgcgtcgtatcgatgtgcaacaggtagaaaaataaggttgctgggaagcggcaggcttcccgtgtatgatgaacccgcccggcgcgacccgttgttcgtcgcggccccgagggttcattttttgtattaataaagagaataaacgtggcaaaaaatattcaagccattcgcggcatgaacgattatctgcctggcg

SEQ ID NO: 11 - DNA, 194 bp, Klebsiella variicola
  source 1..194: mol_type = other DNA
ttcgctaagtcttagcaataaatgagataagcggtgtgtcttgtggaaaaacaaggactaaagcgttacccactaaaaaagatagcgacttttatcactttttagcaaagttgcactggacaaaaggtaccacaattggtgtactgatactcgacacagcattagtgtcgatttttcatataaaggtaattttg

SEQ ID NO: 12 - DNA, 400 bp, Klebsiella variicola
  source 1..400: mol_type = other DNA
gcccgctgaccgaccagaacttccaccttggactcggctatacccttggcgtgacggcgcgcgataactgggactacatccccattccggtgatcttaccattggcgtcaataggttacggtccggcgactttccagatgacctatattcccggcacctacaataacggtaacgtttacttcgcctgggctcgtatacagttttaattcgctaagtcttagcaataaatgagataagcggtgtgtcttgtggaaaaacaaggactaaagcgttacccactaaaaaagatagcgacttttatcactttttagcaaagttgcactggacaaaaggtaccacaattggtgtactgatactcgacacagcattagtgtcgatttttcatataaaggtaattttg

SEQ ID NO: 13 - DNA, 177 bp, Klebsiella variicola
  source 1..177: mol_type = other DNA
tcccggctgtgcgtgagggagactgttcttaatctggcgcgcgaaggttgctattgccctgaaaatggaccaccctagctgaggtcgcacaaaaaacgtgcggccgactttgggttacatttcatccggtcaccaccgggtttgcccttgaaaccagaacaggataaaggagtcaga

SEQ ID NO: 14 - DNA, 400 bp, Klebsiella variicola
  source 1..400: mol_type = other DNA
cccgcagcgggtgatccctggtcattacctcggcaccccgccggagggagacagcgcggtgcgcttcacaaaaacgtatctccagcagtttgagcaggcgctgaagacgcatcaggattcggccggggtgatcaaggccatggagacgcagtggccgggcctggcggagtccagctcgctggagttaagcgccaaagttaataccggcgagatgaagtggtgatcccggctgtgcgtgagggagactgttcttaatctggcgcgcgaaggttgctattgccctgaaaatggaccaccctagctgaggtcgcacaaaaaacgtgcggccgactttgggttacatttcatccggtcaccaccgggtttgcccttgaaaccagaacaggataaaggagtcaga

SEQ ID NO: 15 - DNA, 472 bp, Klebsiella variicola
  source 1..472: mol_type = other DNA
tgagtattgtcagccggaagtttgatcggaaggatgtgggaacggtatttcgccatgcggtgacctgaaaagttttgtgccgtctggaggtaaaactgactgcagaagacagggagcagttactgtccttaatcagtctggtgtatcgcgccggagagaacgccggtagtgaacaacgggcggttgaaatccggcaggcgctgggtttacagacagaaaacgagtcaggaggtgtttgaggatatattcagttatcaggctgttagtcctgggtggattcgatacgacagggtataatgacgtcggcgcttgaggctttttgcctcatgacgtaaaggtggtttgttaccgtgttgtgcggcagaaagcagaaagccccgtagttaattttcattaacccacgaggccccctgtatgtctcatcaacaacagtatggcctcttaccgtgctcaatgcaaggaggagtaaacc

SEQ ID NO: 16 - DNA, 250 bp, Klebsiella variicola
  source 1..250: mol_type = other DNA
agtcaggaggtgtttgaggatatattcagttatcaggctgttagtcctgggtggattcgatacgacagggtataatgacgtcggcgcttgaggctttttgcctcatgacgtaaaggtggtttgttaccgtgttgtgcggcagaaagcagaaagccccgtagttaattttcattaacccacgaggccccctgtatgtctcatcaacaacagtatggcctcttaccgtgctcaatgcaaggaggagtaaacc

SEQ ID NO: 17 - DNA, 100 bp, Klebsiella variicola
  source 1..100: mol_type = other DNA
acgcttcggccgaaaaataagcgcatcggtagcacgctcagtaaatcgccgtctatactgaaagagcctgactgaaggctaattccaaggagattgcagg

SEQ ID NO: 18 - DNA, 250 bp, Klebsiella variicola
  source 1..250: mol_type = other DNA
gagcgcacgccgccgctggcgagcgccgaggtcacggcggcctggatgaatcagattatcgaacagtgcatcctgatggcgcccgagcaatatatgtggctgcaccggcgttttaagactcgcccggaaggggtaccgccgcgttactgaacgcttcggccgaaaaataagcgcatcggtagcacgctcagtaaatcgccgtctatactgaaagagcctgactgaaggctaattccaaggagattgcagg

SEQ ID NO: 19 - DNA, 251 bp, Klebsiella variicola
  source 1..251: mol_type = other DNA
ccagcgcgcagcggcatgggtcagtaagggggcttttgccgctgcaccgtaaaaaaaagtttgctatcaggtgctgaacgtgcgttaatgctcgcaggtttgatgtacagaccacagagcagtcgaatagagcagtccttctaaggttatccaaagatacccccgtagtgaactttccctttatcgctttaaatctgtagtccagaccgctacgccgcaaggctcacttatttttttaaaggtaattcact

SEQ ID NO: 20 - DNA, 350 bp, Klebsiella variicola
  source 1..350: mol_type = other DNA
agtgggagtcgagacgggttagaccgtctcccaccgagctgaaattgatgcgcctgattcaggccaatccacagctttcacgacagttactcgattaatccagcgcgcagcggcatgggtcagtaagggggcttttgccgctgcaccgtaaaaaaaagtttgctatcaggtgctgaacgtgcgttaatgctcgcaggtttgatgtacagaccacagagcagtcgaatagagcagtccttctaaggttatccaaagatacccccgtagtgaactttccctttatcgctttaaatctgtagtccagaccgctacgccgcaaggctcacttatttttttaaaggtaattcact

SEQ ID NO: 21 - DNA, 156 bp, Klebsiella variicola
  source 1..156: mol_type = other DNA
gccggcgatcaaaaaagcagcgatttaatcgttgcatagggcgcgaaattggcatacaatttcgcgccttttgtttttatgggcctggcccgtaaaacgatgtttaatcacggggagcttctctgaagcgttaatacccaatttgaggatttaaga

SEQ ID NO: 22 - DNA, 300 bp, Klebsiella variicola
  source 1..300: mol_type = other DNA
gctaaagttctcggctaatcgctgataacatttgacgcaatgcgcaataaaagggcatcatttgatgccctttttgcacgctttcataccagaacctggctcatcagtgattttttttgtcataatcattgctgagacaggctctgaagagggcgtttatacaccaaaccattcgagcggtagcgcgacggcaagtcagcgttctcctttgcaatagcagggaagaggcgccagaaccgccagcgttgaagcagtttgaacgcgttcagtgtataatccgaaacttaatttcggtttgga

SEQ ID NO: 23 - DNA, 771 bp, Klebsiella variicola
  source 1..771: mol_type = other DNA
cttgtggctgaacgactcatcattgtttgtaaacaggatgtagcgccagagtaactggcaacaaagcagatgctgcaggcagtataaaggctaatggcgtaaatccatactacagaatggtgccagcggcgcgataccctccaggaattatcttagaatcgaagcgcaaatgaaaccgcgccaacaacgctgaccagtcgcgatattgacaaagtacaggcggaagaatcgcacgaaataacaagacattggctgaataagggcaattgacaggctaattgattgattaatagtcgttagggaattttttgccgtagcacagataaattaaagttgtgtaaagaagggtaaaaaaaaccggatgcgaggcatccggttgaaataggggtaaacagacattcagaactgaatgacggtaataaataaagttaatgatgatagcgactgttattttagtcaccaatgatagttttgttttaccattcagtgctatagagttatttgtctgtatgtgattgattgtgaggaaataaatattttttttgattattagtgcgtatttcccagaccattttgtggtgcaaaaagttccgccatttttacaaattgaaacatcttgtgggcattttgaaacatcttagaagttttagtatcatattcttgttggattattctgcattttgcagcacaatgaaatagccgactgattagaagggtaatcagtaagcagtggcataataaaaggcatataacaaacagagggttaataac

SEQ ID NO: 24 - DNA, 249 bp, Klebsiella variicola
  source 1..249: mol_type = other DNA
tgaggaaataaatattttttttgattattagtgcgtatttcccagaccattttgtggtgcaaaaagttccgccatttttacaaattgaaacatcttgtgggcattttgaaacatcttagaagttttagtatcatattcttgttggattattctgcattttgcagcacaatgaaatagccgactgattagaagggtaatcagtaagcagtggcataataaaaggcatataacaaacagagggttaataac

SEQ ID NO: 25 - DNA, 242 bp, Klebsiella variicola
  source 1..242: mol_type = other DNA
ttctgcgagtttcagaaaaaggggcctgacggccccttttttcgaccgggcggcagcaattcattcaaaactcatgtattgttgctagtaatgatcttcatgcagaggttcgcgcggctaatgagaggcttcatccgcaggggcgggtaaaggttgtcattagtcgcgaggatgcagaggatcgggtcaatagacgctatatctttgatatggcgtgatttatagataaaaaggatagaatt

SEQ ID NO: 26 - DNA, 299 bp, Klebsiella variicola
  source 1..299: mol_type = other DNA
cgccgtcctcgcagtaccattgcaaccgactttacagcaagaagtgattctggcacgcatggaacaaattcttgccagtcgggctttatccgatgacgaacgcgcacagcttttatatgagcgcggagtgttgtatgatagtctcggtctgagggcattagcgcgaaatgatttttcacaagcgctggcaatccgacccgatatgcctgaagtattcaattacttaggcatttacttaacgcaggcaggcaattttgatgctgcctatgaagcgtttgattctgtacttgagcttgatc

SEQ ID NO: 27 - DNA, 270 bp, Klebsiella variicola
  source 1..270: mol_type = other DNA
aaacaagggtataaggctatcttgtttgccattttagctccggggtgtgctcgaaatgctcacgtactacgtgtacgctccgctttctgcgcgcacgccggaactaaactagctgcaccgatatacgccttctatcccttgtttaatgctcagtaccaagatgctgattgcattttccccagaaatcagtaaaattttcgggcttttaatatgacaccgggctccgttcctcgatggggcccggttgttttattcacacaagaggatgtt

SEQ ID NO: 28 - DNA, 300 bp, Klebsiella variicola
  source 1..300: mol_type = other DNA
tcggttcagggcaattccattggtctgataaagataatatgtccccgttctcagggggaaaagattgtcgccgcattcaccaaaaatgcgatattccgcgcagggcctccatcttaatacgataaaaggccgctacaagccgttgttacataaccccttcattgtggatctcgcggttaatcgccaaaaatagcgctaaatgacaacaaatatcatttgccttccattcagataatacttacattcataactattagtaatgttttggcgccagggcgctttttatatttcgaggtggat

SEQ ID NO: 29 - DNA, 250 bp, Klebsiella variicola
  source 1..250: mol_type = other DNA
actatcgcgaagacgcgcaaatcccggtgatgattttctaaacagcgcttgcgtcgtgccagaatttgcgtataatgcgcgggcctgtcaaagttgacagccggttcgatatgaaccctgatagtgctttttgctatcaaacaatgtccccaatcgggggactatgtaagaacggttacactctcccatcaatcgtaatgggtatgaggagtaatcatttcgtctataaaataattggagctctggactc

SEQ ID NO: 30 - DNA, 128 bp, Klebsiella variicola
  source 1..128: mol_type = other DNA
atatcgatcaataaatttgaacaatgacagcaaatccttccgctttttgtttagcgatgtgcgggctactatttaacacatcaaggcacggcgccttatctaaacaactaaatgaaagggtttatatc

SEQ ID NO: 31 - DNA, 300 bp, Klebsiella variicola
  source 1..300: mol_type = other DNA
tgaaatggtgcagaaggccgcgatgtgcggcgtcgagatcctgttcgcagtctcggcggccactaccttagcggtggaagtggccgagcgctgcaatctgacgctggtgggcttttgcaagccgggcagggcgacagtctacacccatccgcagcgtttaattgcgggttaaatatcgatcaataaatttgaacaatgacagcaaatccttccgctttttgtttagcgatgtgcgggctactatttaacacatcaaggcacggcgccttatctaaacaactaaatgaaagggtttatatc

SEQ ID NO: 32 - DNA, 352 bp, Klebsiella variicola
  source 1..352: mol_type = other DNA
atgaaattaggattattcctggaattttttttaccgatggtaaagacacagcgtttttcagggactttttcgcgcaatgcctgtcacacggggatttctgccttttttctgcgtacgaaaatcaaccatatttgttaaatattgtgtacacaacccttttttttcatatgcctgacagagttcacacttgtaagtttcgaactaagttgtagactttacatcgccaggggtgatcggcttacgctgcatgtatcagcatagttaacaacaagtcacgccccgggtgaaggatttaaccgtgaggtcttttgtaacttcatggcgaattttggatgataatgaggcgcaaaaa

SEQ ID NO: 33 - DNA, 200 bp, Klebsiella variicola
  source 1..200: mol_type = other DNA
acccttttttttcatatgcctgacagagttcacacttgtaagtttcgaactaagttgtagactttacatcgccaggggtgatcggcttacgctgcatgtatcagcatagttaacaacaagtcacgccccgggtgaaggatttaaccgtgaggtcttttgtaacttcatggcgaattttggatgataatgaggcgcaaaaa

SEQ ID NO: 34 - DNA, 300 bp, Klebsiella variicola
  source 1..300: mol_type = other DNA
gccgacgaagcctcgccgcgccgcttcgttatatacctcaacaggagtactccggttgtatcgataatgcgagggctgcaggtattatttccctgcacacagtaagttagcggtgatgtgccgtctggttatttttaatgtgtgttgtagaattattccgaattactgctgaaagacgtcgggaaaacggaataataatttgactaaccagcattacccgctagagttaaatatcgaacgacgagtgatacggaatattttcgtatcgtactgacataaccgatatacatgaggtgaaat

SEQ ID NO: 35 - DNA, 165 bp, Klebsiella variicola
  source 1..165: mol_type = other DNA
tagagtacgcattctcgatacggataaacggctcagcgatgagccgtttattttttctacccatatctggtttgtggtgttataatgccgcgccctcgatatggggctttttaacgaccctaattttcgggactcagtagtagttgacattagcggagcactaaa

SEQ ID NO: 36 - DNA, 300 bp, Klebsiella variicola
  source 1..300: mol_type = other DNA
acgaccaaactgcacgtacatgacgagaacaacgaatgcggtatcggtgacgtggttgaaatccgcgaatgccgtccgctgtccaagactaagtcctggacgctggttcgcgttgtagagaaagcggttctgtaatagagtacgcattctcgatacggataaacggctcagcgatgagccgtttattttttctacccatatctggtttgtggtgttataatgccgcgccctcgatatggggctttttaacgaccctaattttcgggactcagtagtagttgacattagcggagcactaaa

SEQ ID NO: 37 - DNA, 71 bp, Klebsiella variicola
  source 1..71: mol_type = other DNA
gaatttacttacattaaggcggcgaggggcgcctatacttgatagttctgataccagaagaaggaagaact

SEQ ID NO: 38 - DNA, 151 bp, Klebsiella variicola
  source 1..151: mol_type = other DNA
atgccacggcctccccggatcgggtggtggagcagattatgaccatgctgtgcggcgcgacggcaaccccggtaagttaagaatttacttacattaaggcggcgaggggcgcctatacttgatagttctgataccagaagaaggaagaact

SEQ ID NO: 39 - DNA, 364 bp, Klebsiella variicola
  source 1..364: mol_type = other DNA
taactataaacgcctataccctaaataattcgagtggcaggaaggcggcgacgcagcgaatccccaggagcttactcaagtaagtgactggggtgagtgaggaaagccaacacacaggcaacttgaagtatggcgggtataggtgccgtaacctcgggggaacggcaccttgcgtcataagtactgataacgataaagtcgggttgaaattgtgtatatcggctaaacttaggtttaacagaatgtgatgccatgactgccttataccgcaaggtatttgtcatcgcttactttttggcgttatatgatggataatgccgggatacgagagtcccgactcttttaatctttcaaggagcaaaga

SEQ ID NO: 40 - DNA, 200 bp, Klebsiella variicola
  source 1..200: mol_type = other DNA
gcaccttgcgtcataagtactgataacgataaagtcgggttgaaattgtgtatatcggctaaacttaggtttaacagaatgtgatgccatgactgccttataccgcaaggtatttgtcatcgcttactttttggcgttatatgatggataatgccgggatacgagagtcccgactcttttaatctttcaaggagcaaaga

SEQ ID NO: 41 - DNA, 204 bp, Rahnella aquatilis
  source 1..204: mol_type = other DNA
agtattaaaggcggaaaacgagttcaaccggcgcgtcctaatcgcattaacaaagagattcgcgcgcaagaagttcgcctcacaggcgtcgatggcgagcagattggtattgtcagtctgaatgaagctcttgaaaaagctgaggaagcgggcgtcgatttagtagaaatcagtccgaatgccgagccgccagtttgtcgaatc

SEQ ID NO: 42 - DNA, 500 bp, Rahnella aquatilis
  source 1..500: mol_type = other DNA
tgaacatcactgatgcacaagctacctatgtcgaagaattaactaaaaaactgcaagatgcaggcattcgcgttaaagccgacttgagaaatgagaagattggctttaaaattcgcgaacacacgctacgccgtgttccttatatgttagtttgtggcgataaagaggtcgaagcaggcaaagttgctgttcgtacccgccgcggcaaagacttaggaagcatggatgttagcgaagtcgttgacaaactgctggcggaaatccgcagcagaagtcttcatcaactggaggaataaagtattaaaggcggaaaacgagttcaaccggcgcgtcctaatcgcattaacaaagagattcgcgcgcaagaagttcgcctcacaggcgtcgatggcgagcagattggtattgtcagtctgaatgaagctcttgaaaaagctgaggaagcgggcgtcgatttagtagaaatcagtccgaatgccgagccgccagtttgtcgaatc

SEQ ID NO: 43 - DNA, 120 bp, Rahnella aquatilis
  source 1..120: mol_type = other DNA
tctggccttaatctggtgctgaagaatattcagtgccggttttggctatagttttttttaacctcgccgcaaggatctgtagcggggcatttgaaacaaccccatccagcaggacgccag

SEQ ID NO: 44 - DNA, 400 bp, Rahnella aquatilis
  source 1..400: mol_type = other DNA
tgcaccggtgaagatatttctggatgccagttcggaagaacgtgcaaacagaagaatgctacagttgcaggaaaaaggctttagtgttaactttgaacggcttttagccgagatcaaagaacgcgatgaccgtgatcgtaacaggcctatcgcgcctttagtggctgcttccgatgcactgttgctggattcaaccagtatgtctatcgacgaagtcatcgaaaaagcactggcttatgccacagaaattctaggattaccgcaaaaacaaacccggtaatctggccttaatctggtgctgaagaatattcagtgccggttttggctatagttttttttaacctcgccgcaaggatctgtagcggggcatttgaaacaaccccatccagcaggacgccag

SEQ ID NO: 45 - DNA, 170 bp, Rahnella aquatilis
  source 1..170: mol_type = other DNA
tacagtagcgcctctcaaaaatagataaacggctcatgtacgtgggccgtttattttttctacccataatcgggaaccggtgttataatgccgcgccctcatattgtggggatttcttaatgacctatcctgggtcctaaagttgtagttgacattagcggagcactaac

SEQ ID NO: 46 - DNA, 129 bp, Rahnella aquatilis
  source 1..129: mol_type = other DNA
tctgtaacagaagttttacagctcctttccatctggaaaggagctgttcgtctcacggacgcaggacgcgtttgtgttaagcaagcggatgacaggatgttcatccaatgtttgtctccgggagtagaa

SEQ ID NO: 47 - DNA, 273 bp, Rahnella aquatilis
  source 1..273: mol_type = other DNA
tcaagcgagtttcagtgtaaaggggccaataggcccctttattctaggaagcgcagccaaatcagggtactgtatggctgcggtttctactgttattctaagaacatgaacttccgttacagatgttttcgcgcggctaatgagagactttattaccacattgccaggtatataaggattgtcattagtcgcgagaatgtagtgagaagctcggatatttatcggcgtgaactgctgtcataacagctgcgcgtcatacaaaaggatattaca

SEQ ID NO: 48 - DNA, 153 bp, Rahnella aquatilis
  source 1..153: mol_type = other DNA
aaattacgaaattatttgcgttttttgcggtaaaaaccgcaaaatagagcaaattcgtggtttgaccagcctggatttagttgcatctttttcaacattttatacactacgaaaaccatcgcgaaagcgagttttgataggaaatttaagagt

SEQ ID NO: 49 - DNA, 750 bp, Rahnella aquatilis
  source 1..750: mol_type = other DNA
gaatatttaggcgaaaatggcaagggtatcatgctcaatgtggttgattctgcatctattgagcaagtattggcgacgattcgagctgaatttggcgaaattgatattttagttaataatgccggcatcacccgtgataaccttctcatgcgtatgaaggatgatgagtggcaggatatcctggatacgaacctgacttcagtgtttcggctgtcaaaagctgtcatgcgagctatgatgaagaaacggtgtggacggattattacaattggttccgttgttggcaccatgggtaacgcagggcaggcgaactacgcggcggctaaagctggcttgattggttttagtaagtctttggcacgtgaggtcgcttcacgtggcattactgtcaacgtcgtggctcccggctttattgagacggatatgacaagggcgttgacagatgatcaacgcgcaggcattttgtcatcagttccagccaaccggttgggcgatgccaaagaaattgccagcgccgttgcttttttagcctctgacgaggccagctacatcacgggtgaaacattacatgtcaatggcggcatgtatatgatttaaaaattacgaaattatttgcgttttttgcggtaaaaaccgcaaaatagagcaaattcgtggtttgaccagcctggatttagttgcatctttttcaacattttatacactacgaaaaccatcgcgaaagcgagttttgataggaaatttaagagt

SEQ ID NO: 50 - DNA, 142 bp, Rahnella aquatilis
  source 1..142: mol_type = other DNA
aattttttttcacaaagcgtagcgttattgaatcgcacattttaaactgttggccgctgtggaagcgaatattggtgaaaggtgcggttttaaggcctttttctttgactctctgtcgttacaaagttaatatgcgcgccct

SEQ ID NO: 51 - DNA, 293 bp, Rahnella aquatilis
  source 1..293: mol_type = other DNA
ttaaaaacgtgaccacgagcattaataaacgccacgaaatgtggcgtttatttattcaaaaagtatcttctttcataaaaagtgctaaatgcagtagcagcaaaattgggataagtcccatggaatacggctgttttcgctgcaatttttaactttttcgtaaaaaaagatgtttctttgagcgaacgatcaaaatatagcgttaaccggcaaaaaattattctcattagaaaatagtttgtgtaatacttgtaacgctacatggagattaacttaatctagagggttttata

SEQ ID NO: 52 - DNA, 198 bp, Rahnella aquatilis
  source 1..198: mol_type = other DNA
acaccctccttccatcaccattgtgattatggttattaaatttttatagaaataacttaacgatcattattaaaaatagttgcgcacaagtccagcggagtttattatttaattatcgagcgataagaaaatcgctcaaacccgcaaaactgcgcagctaaaaacgctttttaagcatactatccaggacgtaacatc

SEQ ID NO: 53 - DNA, 285 bp, Rahnella aquatilis
  source 1..285: mol_type = other DNA
taaaaaattcctgaacgggcggtaaatgaaaaaggttttatcaatcattcatgctgtgagcacggtttgcaaggcttgcagtatgaattgatgcaacaatgtgtggtgaccagaaatcactgccggttcattcagataggtcaaaggtatcggactgacaggtaattcctgcttttttttatgatctgcaacaggcatagtatcgacatcaaaaaggtgatgtggataacaaaaaacaaacattcccttttcatttatctcgttggcattaacaaaggagtctcc

SEQ ID NO: 54 - DNA, 224 bp, Rahnella aquatilis
  source 1..224: mol_type = other DNA
tcaatctcatcagttctgtgaaccgtcccgcaattccctgcaatacaagaggttgttgttaaagaactctgagacttacgtcaaagactgatagccggatactatctgattgattggtgcgatggggtttattcacccgcagcttgcccctatactgacagtcgttttgttcatcctttcctttcacctacgacgccctcttgggtttcataaggagtaatatt

SEQ ID NO: 55 - DNA, 1256 bp, Rahnella aquatilis
  source 1..1256: mol_type = other DNA
taccggcggtttgcaaccaggtaaagacatccggccactgccagcttgattcatcgataatcacctgcgtgttgtccggcaatacgcgggggatattttcccagaagccgccaccggtcagatggacgatgccgtgaacatccacgttttcgatcaggttcaggatcgatttcacgtaaattttggtcggtgcgagcaaatgatcagccagcggtttgcctgccagatcggtggtttccgggtcggtcttgctgacttccagaattttgcgcaccagagaataaccgttagaatgcgggccactggcggccagaccaatcagcacatccccgtcagccactttgctgccgtcgatgatttctgatttttccaccacgcccacgcagaagcctgccacgtcgtaatcttcgccgtgatacatgcccggcatttcagcggtttcaccgccaactaacgcacagccagactgtttacagccttctgcgatacccgtgatcacgctggcagccgtatcgacgtccagtttgccggtagcgtaataatcgaggaagaacaggggttcggcgccctgaacgatcaaatcgttgacgcacatcgcgaccaggtcgataccgatagtatcgtggcgtttcaaatccatcgccagacgcagcttggtgccaacgccgtcggtacccgataccagcacgggttcacgatatttttgcggcagcgcgcagagggcaccaaaaccgcccagtccacccatgacttcagggcggcgagtctgttttactacacctttaatgcggtctaccaatgcgttaccggcatcgatatctacgcctgcgtctttatagctgagagaggttttgtcggtcactgcgaagtccccacggcggtttgggttggtggttgaagaataaagcggggcaattctaacagtgcaagcaaacgtttgcgagcgccttattcagagtcactatctatacttaaaaatacaacacttagccgaagtcattggagttgcagcaaggcagcaaacgagcgaatcccgatgagctgacttgagtcagtgattcgggtgagagagagcagctaacgcagctgcggcttcaatgaagcagggtaagttgatccagatcaggctatttggtatggcgttcaaaaaaaatggcgttataatctcgcgatttttttttgcagctcaaccaccttaggagaataaataatgaagatcgtcgaggtgaaacacccgctggtgaaacacaagctgg

SEQ ID NO: 56 - DNA, 449 bp, Rahnella aquatilis
  source 1..449: mol_type = other DNA
cggcatcgatatctacgcctgcgtctttatagctgagagaggttttgtcggtcactgcgaagtccccacggcggtttgggttggtggttgaagaataaagcggggcaattctaacagtgcaagcaaacgtttgcgagcgccttattcagagtcactatctatacttaaaaatacaacacttagccgaagtcattggagttgcagcaaggcagcaaacgagcgaatcccgatgagctgacttgagtcagtgattcgggtgagagagagcagctaacgcagctgcggcttcaatgaagcagggtaagttgatccagatcaggctatttggtatggcgttcaaaaaaaatggcgttataatctcgcgatttttttttgcagctcaaccaccttaggagaataaataatgaagatcgtcgaggtgaaacacccgctggtgaaacacaagctgg

SEQ ID NO: 57 - DNA, 312 bp, Rahnella aquatilis
  source 1..312: mol_type = other DNA
cgcggtttggttggtcaaatttcacacaaaatcacgttgatcgactatactggtttcgtcgcgctgactgagaaacatgcccagcaaacagcatggtgaaacatcgatgtgctgtatatttcttgacaccctcttaggtcagccctaaaattctgcgtccccatattagctaatgctttttatggggcgatttatcacgcgtttacaaagtagtttatgaaccaaaatccaggagctttttaatggcaacaattaatcagctggtacgcaaaccacgctctacgaaggttgctaaaagcaacgttccagcgc

SEQ ID NO: 58 - DNA, 489 bp, Rahnella aquatilis
  source 1..489: mol_type = other DNA
cctggttgagtctgctccagcagctctgaaagaaggcatcagcaaagatgacgctgaagctctgaaaaaatctctggaagaagctggtgcttctgttgaagttaagtaagtttaacttcccggagtgcagtctgtcctaacaggctgatggctggtgactttttagtcaccagcctttttgcgctatagagtgtcagtgatgtttcacactgtttgagcactgaactactctaatatctctttctatagacgccttaatatattgttgcctcttgctgtagctcatctacagataacgcacaacgaaatgatttaagagtggtagaaaacagatattgcggaaagcgtttctgctttccggtcgacataaacggtgttgcatgaactgtccttctcagggcagacaagattgggtcactgatcagcgagctgaggaaccctatggtttactcctataccgagaaaaaacgcattcgtaaggattttggt

SEQ ID NO: 59 - DNA, 498 bp, Rahnella aquatilis
  source 1..498: mol_type = other DNA
tgaatatcactgactcacaagctacctatgtcgaagaattaactaaaaaactgcaagatgcaggcattcgcgttaaagccgacttgagaaatgagaagattggctttaaaattcgcgaacacacgctacgccgtgttccttatatgttagtttgtggcgataaagaggtcgaagcaggcaaagttgctgttcgtactcgtcgcggcaaagacttaggaagcatggatgttagcgaagtcgttacaaactgcggcggaaatccgcagcagaagtcttcatcaactggaggaataaagtattaaaggcggaaaacgagttcaaccggcgcgtcctaatcgcattaacaaagagattcgcgcgcaagaagttcgcctcaccggcgtcgatggcgagcagattggtattgtcagtctgaatgaagctcttgaaaaagctgaggaagcgggcgtcgatttagtagaaatcagtccgaatgccgagccgccagtttgtcgaatc

SEQ ID NO: 60 - DNA, 498 bp, Kluyvera intermedia
  source 1..498: mol_type = other DNA
ctggggtcactggagcgctttatcggcatcctgaccgaagaatttgccggtttcttcccgacctggctggcccctgttcaggttgtggtgatgaatatcactgattctcaagctgaatatgtcaacgaattgacccgtaaattgcaaaatgcgggcattcgtgtaaaagcggacttgagaaacgagaagattggctttaaaatccgcgagcacactttacgtcgtgtcccttatatgttggtctgtggtgataaagaggtggaagcaggcaaagtggccgttcgcacccgccgcggtaaagacctgggcagcctggacgtaagtgaagtgattgagaagctgcaacaagagattcgcagccgcagtcttcaacaactggaggaataaggtattaaaggcggaaaacgagttcaaacggcacgtccgaatcgtatcaatggcgagattcgcgcccaggaagttcgcttaactggtctggaaggtgagcagctgggtatt

SEQ ID NO: 61 - DNA, 500 bp, Kosakonia pseudosacchari
  source 1..500: mol_type = other DNA
ttcttggttctctggagcgctttatcggcatcctgactgaagaatttgcaggcttcttcccaacctggcttgcacccgtgcaggtagttgtgatgaacatcactgattcgcaggctgaatacgttaacgaattgacccgtaaactgcaaaatgcgggcattcgtgtaaaagcagacttgagaaacgagaagattggctttaaaatccgcgagcacactttacgtcgtgtcccttatatgctggtttgtggtgacaaagaggtcgaagccggcaaagttgctgtgcgtacccgtcgcggtaaagacctgggtagcctggacgtaaatgatgttatcgagaagctgcaacaagagattcgcagccgcagtcttcaacaactggaggaataaggtattaaaggcggaaaacgagttcaaacggcgcgtcccaatcgtattaatggcgagattcgcgccacggaagttcgcttaacaggtctggaaggcgagcagcttggtatt

SEQ ID NO: 62 - DNA, 500 bp, Enterobacter sp.
  source 1..500: mol_type = other DNA
tgaatattaccgattctcaggcggattacgttaaagaattgacgcagaaacttcaaaatgcgggcattcgcgtaaaagcagacttgagaaatgagaagattggctttaaaatccgcgagcacactttacgtcgtgtcccgtatatgttggtctgtggtgataaagaggtggaagcaggcaaagttgccgttcgcacccgccgtggtaaagacctgggcagcctggacgtaagtgaagtgattgagaagctgcaacaagagattcgcagccgcagtcttcaacaactggaggaataaggtattaaaggcggaaaacgagttcaaacggcacgtccgaatcgtatcaatggcgagattcgcgcccaggaagttcgcttaacagatcttgaaggtgaaccactggggattgtgagtctgagagaagcgatcgaaaaagctgaagaagctggagtagatttagttgaaatcagccctaacgccgaaccgccagtttgtcgtatt

SEQ ID NO: 63 - DNA, 500 bp, Rahnella aquatilis
  source 1..500: mol_type = other DNA
tgaatatcactgactcacaagctacctatgtcgaagaattaactaaaaaactgcaagatgcaggcattcgcgttaaagccgacttgagaaatgagaagattggctttaaaattcgcgaacacacgctacgccgtgttccttatatgttagtttgtggcgataaagaggtcgaagcaggcaaagttgctgttcgtactcgtcgcggcaaagacttaggaagcatggatgttagcgaagtcgttgacaaactgctggcggaaatccgcagcagaagtcttcatcaactggaggaataaagtattaaaggcggaaaacgagttcaaccggcgcgtcctaatcgcattaacaaagagattcgcgcgcaagaagttcgcctcaccggcgtcgatggcgagcagattggtattgtcagtctgaatgaagctcttgaaaaagctgaggaagcgggcgtcgatttagtagaaatcagtccgaatgccgagccgccagtttgtcgaatc

SEQ ID NO: 64 - DNA, 170 bp, Rahnella aquatilis
  source 1..170: mol_type = other DNA
tacagtagcgcctctcaaaaatagataaacggctcatgtacgtgggccgtttattttttctacccataatcgggaaccggtgttataatgccgcgccctcatattgtggggatttcttaacgacctatcctgggtcctaaagttgtagttgacattagcggagcactaac

SEQ ID NO: 65 - DNA, 142 bp, Rahnella aquatilis; identical to SEQ ID NO: 50
  source 1..142: mol_type = other DNA

SEQ ID NO: 66 - DNA, 293 bp, Rahnella aquatilis
  source 1..293: mol_type = other DNA
ttaaaaacgtgaccacgagcattaatgaacgctgcgaaatgtggcgtttatttattcaaaaagtatcttctttcataaaaagtgctaaatgcagtagccgcaaaattgggataagtcccatggaatacggctgttttcgctgcaatttttaactttttcgtaaaaaaagatgcttctttgagcgaacgatcaaaatatagcgcttaccgacaaaaaattattctcattagaaaatagtttgtgtaatacttgtaacgctacatggagattaacttaatctagagggttttata

SEQ ID NO: 67 - DNA, 500 bp, Klebsiella sp.
  source 1..500: mol_type = other DNA
agcgtcaggtaccggtcatgattcaccgtgcgattctcggttccctggagcgcttcattggcatcctgaccgaagagttcgctggcttcttcccaacctggattgcaccagtgcaggtagtggtcatgaatattaccgattctcaggctgaatacgttaacgaattgacgcgtaaactacaaaatgcgggcattcgtgtaaaagcagacttgagaaatgagaagattggctttaaaatccgcgagcacactttacgtcgtgtcccgtatatgttggtctgtggcgacaaagaagtcgaagccggcaaagtggccgtgcgcacccgtcgcgggaaagacctcggcagcatggacgtaagtgaagtgattgagaagctgcaacaagagattcgcagccgcagtcttcaacaactggaggaataaggtattaaaggcggaaaacgagttcaaacggcacgtccgaatcgtatcaatggcgagattcgcgccctggaagttcgc

SEQ ID NO: 68 - DNA, 400 bp, Klebsiella sp.; identical to SEQ ID NO: 12
  source 1..400: mol_type = other DNA

SEQ ID NO: 69 - DNA, 300 bp, Klebsiella sp.; identical to SEQ ID NO: 22
  source 1..300: mol_type = other DNA

SEQ ID NO: 70 - DNA, 333 bp, Kosakonia pseudosacchari
  variation 56: note = a, c, t, g, unknown, or other
  source 1..333: mol_type = other DNA
tcgccacggcgataaccataggttttcggcgtggccacatccatggtaaatcccantttttccagcacgcgcgccacttcatcgggtcttaaatacatagattttcctcgtcatctttccaaagcctcgccaccttacatgactgagcatggaccgtgactcagaaaattccacaaacgaacctgaaaggcgtgattgccgtctggccttaaaaattatggtctaaactaaaattcacatcgaaaacgagggaggatcctatgtttaacagaccgaatcgccgtgacgtagatgaaggtgttgaggatattaaccacgatgttaaccagctcg

SEQ ID NO: 71 - DNA, 339 bp, Kosakonia pseudosacchari; identical to SEQ ID NO: 7
  source 1..339: mol_type = other DNA

SEQ ID NO: 72 - DNA, 350 bp, Kosakonia pseudosacchari
  source 1..350: mol_type = other DNA
atattgacacaatgacgcgcgtactgctgattggttctgtgacgctggtgatgattgtcgaaattctgaacagcgccatcgaagctgtggtagaccgtattggtgcggaattccatgaactttccgggcgggcgaaggatatggggtcggcggcggtgctgatgtccatcctgctggcgctgtttacctggatcgcattactctggtcacattttggataacgcttccagaattcgataacgccctggttttttgcttaaatttggttccaaaatcgcctttagctgtatatactcacagcataactgtatatacacccagggggcgggatgaaagcattaacggccagg

SEQ ID NO: 73 - DNA, 90 bp, synthetic construct
  misc_feature 1..90: note = Description of Artificial Sequence: Synthetic oligonucleotide
  source 1..90: mol_type = other DNA
gttgatcagaccgatgttcggaccttccaaggtttcgatcggacatacgcgaccgtagtgggtcgggtgtacgtctcgaacttcaaagcc

(19) United States
(12) Patent Application Publication
(10) Pub. No.: US 2024/0327851 A1
(43) Pub. Date: Oct. 3, 2024

(54) METHODS AND COMPOSITIONS FOR IMPROVING ENGINEERED MICROBES THAT FIX NITROGEN

(71) Applicant: Pivot Bio, Inc., Berkeley, CA (US)

(72) Inventors: Alvin Tamsir, San Francisco, CA (US); Sarah Bloch, Emeryville, CA (US); Douglas Higgins, Berkeley, CA (US)

(21) Appl. No.: 18/632,573

(22) Filed: Apr. 11, 2024

Related U.S. Application Data
(62) Division of application No. 16/759,212, filed on Apr. 24, 2020, now Pat. No. 11,993,778.
(60) Provisional application No. 62/577,148, filed on Oct. 25, 2017.

(51) Int. Cl. C12N 15/82; C07K 14/195; C12N 9/02; C12N 15/10

(52) U.S. Cl. CPC C12N 15/8202; C07K 14/195; C12N 9/0095; C12N 15/102; C12N 15/8262; C12Y 118/06001

(57) ABSTRACT

Methods and systems are provided for generating and utilizing a bacterial composition that comprises at least one genetically engineered bacterial strain that fixes atmospheric nitrogen in an agricultural system that has been fertilized with more than 20 lbs of Nitrogen per acre.

### CROSS-REFERENCE

This application is a divisional of U.S. application Ser. No. 16/759,212, filed on Apr. 24, 2020, which is a National Stage application under 35 U.S.C. § 371 of International Application No. PCT/US2018/057174 having an International Filing Date of Oct. 23, 2018, which claims priority to U.S. Provisional Patent Application No. 62/577,148, filed Oct. 25, 2017, each of which is entirely incorporated herein by reference.

### STATEMENT AS TO FEDERALLY SPONSORED RESEARCH

This invention was made with the support of the United States government under SBIR grant 1520545 awarded by the National Science Foundation. The government has certain rights in the disclosed subject matter.

### SEQUENCE LISTING

This application contains a Sequence Listing that has been submitted electronically as an XML file named 48624-0011002_ST26_SL.XML.” The XML file, created on Jun. 24, 2024, is 88,058 bytes in size. The material in the XML file is hereby incorporated by reference in its entirety.

### BACKGROUND OF THE INVENTION

Plants are linked to the microbiome via a shared metabolome. A multidimensional relationship between a particular crop trait and the underlying metabolome is characterized by a landscape with numerous local maxima. Optimizing from an inferior local maximum to another representing a better trait by altering the influence of the microbiome on the metabolome may be desirable for a variety of reasons, such as for crop optimization. Economically-, environmentally-, and socially-sustainable approaches to agriculture and food production are required to meet the needs of a growing global population. By 2050 the United Nations' Food and Agriculture Organization projects that total food production must increase by 70% to meet the needs of the growing population, a challenge that is exacerbated by numerous factors, including diminishing freshwater resources, increasing competition for arable land, rising energy prices, increasing input costs, and the likely need for crops to adapt to the pressures of a drier, hotter, and more extreme global climate.

One area of interest is in the improvement of nitrogen fixation. Nitrogen gas (N₂) is a major component of the atmosphere of Earth. In addition, elemental nitrogen (N) is an important component of many chemical compounds which make up living organisms. However, many organisms cannot use N₂ directly to synthesize the chemicals used in physiological processes, such as growth and reproduction. In order to utilize the N₂, the N₂ must be combined with hydrogen. The combining of hydrogen with N₂ is referred to as nitrogen fixation. Nitrogen fixation, whether accomplished chemically or biologically, requires an investment of large amounts of energy. In biological systems, an enzyme known as nitrogenase catalyzes the reaction which results in nitrogen fixation. An important goal of nitrogen fixation research is the extension of this phenotype to non-leguminous plants, particularly to important agronomic grasses such as wheat, rice, and maize. Despite enormous progress in understanding the development of the nitrogen-fixing symbiosis between rhizobia and legumes, the path to use that knowledge to induce nitrogen-fixing nodules on non-leguminous crops is still not clear. Meanwhile, the challenge of providing sufficient supplemental sources of nitrogen, such as in fertilizer, will continue to increase with the growing need for increased food production.

### SUMMARY OF THE INVENTION

In some embodiments, the present disclosure provides a genetically engineered bacterium comprising an inserted sequence having at least about 80% sequence identity to a sequence selected from the group consisting of: SEQ ID Nos. 1-72. In some cases, the inserted sequence replaces a native promoter sequence. In some cases, the inserted sequence comprises at least about 85% sequence identity to a sequence selected from the group consisting of: SEQ ID Nos. 1-72. In some cases, the inserted sequence comprises at least about 90% sequence identity to a sequence selected from the group consisting of: SEQ ID Nos. 1-72. In some cases, the inserted sequence comprises at least about 95% sequence identity to a sequence selected from the group consisting of: SEQ ID Nos. 1-72. In some cases, the inserted sequence comprises at least about 97% sequence identity to a sequence selected from the group consisting of: SEQ ID Nos. 1-72. In some cases, the said inserted sequence comprises at least about 98% sequence identity to a sequence selected from the group consisting of: SEQ ID Nos. 1-72. In some cases, the inserted sequence comprises at least about 99% sequence identity to a sequence selected from the group consisting of: SEQ ID Nos. 1-72.

In some embodiments, the present disclosure provides a genetically engineered bacterium, comprising a native coding sequence operably linked to an inserted sequence with at least 80% sequence identity to a sequence selected from the group consisting of: SEQ ID Nos. 1-72. In some cases, the inserted sequence replaces anative promoter sequence. In some cases, the inserted sequence comprises at least about 85% sequence identity to a sequence selected from the group consisting of: SEQ ID Nos. 1-72. In some cases, the sequence comprises at least about 90% sequence identity to a sequence selected from the group consisting of: SEQ ID Nos. 1-72. In some cases, the inserted sequence comprises at least about 95% sequence identity to a sequence selected from the group consisting of: SEQ ID Nos. 1-72. In some cases, the inserted sequence comprises at least about 97% sequence identity to a sequence selected from the group consisting of: SEQ ID Nos. 1-72. In some cases, the said inserted sequence comprises at least about 98% sequence identity to a sequence selected from the group consisting of: SEQ ID Nos. 1-72. In some cases, the inserted sequence comprises at least about 99% sequence identity to a sequence selected from the group consisting of: SEQ ID Nos. 1-72. In some cases, the native coding sequence is selected from the group consisting of: cysZ, otsB, a bcs gene, and treZ. In some cases, the native coding sequence is selected from the group consisting of: a transporter gene, an ion transporter gene, an exopolysaccharide biosynthesis gene, a cellulose biosynthesis gene, and a trehalose biosynthesis gene.

In some embodiments, the present disclosure provides a genetically engineered bacterium, comprising a nitrogen fixation or nitrogen assimilation coding sequence operably linked to an inserted sequence having at least about 80% sequence identity to a sequence selected from the group consisting of: SEQ ID Nos. 1-72. In some cases, the inserted sequence having at least about 80% sequence identity to a sequence selected from the group consisting of: SEQ ID Nos. 1-72 replaces a native promoter sequence. In some cases, the inserted sequence comprises at least about 85% sequence identity to a sequence selected from the group consisting of: SEQ ID Nos. 1-72. In some cases, the inserted sequence comprises at least about 90% sequence identity to a sequence selected from the group consisting of: SEQ ID Nos. 1-72. In some cases, the inserted sequence comprises at least about 95% sequence identity to a sequence selected from the group consisting of: SEQ ID Nos. 1-72. In some cases, the inserted sequence comprises at least about 97% sequence identity to a sequence selected from the group consisting of: SEQ ID Nos. 1-72. In some cases, the inserted sequence comprises at least about 98% sequence identity to a sequence selected from the group consisting of: SEQ ID Nos. 1-72. In some cases, the inserted sequence comprises at least about 99% sequence identity to a sequence selected from the group consisting of: SEQ ID Nos. 1-72. In some cases, the nitrogen fixation or nitrogen assimilation coding sequence is selected from the group consisting of: nifA, nifL, ntrB, ntrC, polynucleotide encoding glutamine synthetase, glnA, glnB, glnK, drat, amtB, polynucleotide encoding glutaminase, glnD, glnE, nifJ, nifH, nifD, nifK, nifY, nifE, nifN, nifU, nifS, nifV, nifW, nifZ, nifM, nifF, nifB, nifQ, and a gene associated with biosynthesis of a nitrogenase enzyme. In some cases, the genetically engineered bacterium is a genetically engineered diazotrophic bacterium. In some cases, the genetically engineered bacterium is non-intergeneric. In some cases, the genetically engineered bacterium is intergeneric. In some cases, the genetically engineered bacterium fixes atmospheric nitrogen under non nitrogen limiting conditions. In some cases, the genetically engineered bacterium fixes more atmospheric nitrogen than a non-engineered bacterium of the same species. In some cases, the genetically engineered bacterium is selected from the group consisting of Rahnella aquatilis, Klebsiella variicola, Kosakonia pseudosacchari, Kluyvera intermedia, Klebsiella sp., Enterobacter sp., and Kosakonia sacchari. In some cases, the genetically engineered bacterium is of the genus Rahnella and said inserted sequence comprises at least about 80% sequence identity to a sequence selected from the group consisting of: SEQ ID Nos. 41-59, and 63-66. In some cases, the genetically engineered bacterium is Rahnella aquatilis and said inserted sequence comprises at least about 80% sequence identity to a sequence selected from the group consisting of: SEQ ID Nos. 41-59, and 63-66. In some cases, the genetically engineered bacterium is of the genus Kosakonia and said inserted sequence comprises at least about 80% sequence identity to a sequence selected from the group consisting of: SEQ ID Nos. 1-10. In some cases, the genetically engineered bacterium is Kosakonia sacchari and said inserted sequence comprises at least about 80% sequence identity to a sequence selected from the group consisting of: SEQ ID Nos. 1-10. In some cases, the genetically engineered bacterium is of the genus Klebsiella and said inserted sequence comprises at least about 80% sequence identity to a sequence selected from the group consisting of: SEQ ID Nos. 11-40. In some cases, the said genetically engineered bacterium is Klebsiella variicola and said inserted sequence comprises at least about 80% sequence identity to a sequence selected from the group consisting of: SEQ ID Nos. 11-40. In some cases, the genetically engineered bacterium is of the genus Kluyvera and said inserted sequence comprises at least about 80% sequence identity to SEQ ID NO. 60. In some cases, the genetically engineered bacterium is Kluyvera intermedia and said inserted sequence comprises at least about 80% sequence identity to SEQ ID NO. 60. In some cases, the genetically engineered bacterium is Kosakonia pseudosacchari and said inserted sequence comprises at least about 80% sequence identity to a sequence selected from the group consisting of: SEQ ID Nos. 61, and 70-72. In some cases, the genetically engineered bacterium is an Enterobacter species and said inserted sequence comprises at least about 80% sequence identity to SEQ ID NO. 62. In some cases, the genetically engineered bacterium is a Klebsiella species and said inserted sequence comprises at least about 80% sequence identity to a sequence selected from the group consisting of: SEQ ID Nos. 67-69. In some cases, the inserted sequence is a native sequence inserted in a non-native context.

In some embodiments, the present disclosure provides a composition comprising a plant seed and a genetically engineered bacterium described herein. In some cases, the plant seed is selected from the group consisting of: corn seeds, wheat seeds, rice seeds, barley seeds, soy seeds, sorghum seeds, and rye seeds.

In some embodiments, the present disclosure provides a composition comprising a plant and a genetically engineered bacterium provided herein. In some cases, the plant is a seedling. In some cases, the plant is selected from the group consisting of corn, wheat, rice, barley, rye, soy, and sorghum.

In some embodiments, the present disclosure provides a method of increasing expression of a microbial gene in a microbe by replacing a native promoter sequence of the microbial gene with a promoter sequence comprising at least 80% sequence identity to a sequence selected from the group consisting of: SEQ ID Nos. 1-72. In some cases, the promoter sequence comprises at least 85% sequence identity to a sequence selected from the group consisting of: SEQ ID Nos. 1-72. In some cases, the promoter sequence comprises at least 90% sequence identity to a sequence selected from the group consisting of: SEQ ID Nos. 1-72. In some cases, the promoter sequence comprises at least 95% sequence identity to a sequence selected from the group consisting of: SEQ ID Nos. 1-72. In some cases, the promoter sequence comprises at least 97% sequence identity to a sequence selected from the group consisting of: SEQ ID Nos. 1-72. In some cases, the promoter sequence comprises at least 98% sequence identity to a sequence selected from the group consisting of: SEQ ID Nos. 1-72. In some cases, the promoter sequence comprises at least 99% sequence identity to a sequence selected from the group consisting of: SEQ ID Nos. 1-72. In some cases, increasing expression of said microbial gene increases ammonium excretion by said microbe. In some cases, increasing expression of said microbial gene increases nitrogen fixation by said microbe. In some cases, increasing expression of said microbial gene increases colonization of a plant by said microbe.

In some embodiments, the present disclosure provides polynucleotide comprising a coding sequence for a protein related to nitrogen fixation or assimilation and a sequence comprising at least 80% sequence identity to a sequence selected from the group consisting of: SEQ ID Nos. 1-72. In some cases, the coding sequence for a protein related to nitrogen fixation or assimilation is selected from the group consisting of: nifA, nifL, ntrB, ntrC, polynucleotide encoding glutamine synthetase, glnA, glnB, glnK, drat, amtB, polynucleotide encoding glutaminase, glnD, glnE, nifJ, nifH, nifD, nifK, nifY, nifE, nifN, nifU, nifS, nifV, nifW, nifZ, nifM, nifF, nifB, nifQ, and a gene associated with biosynthesis of a nitrogenase enzyme.

In some embodiments, the present disclosure provides a polynucleotide comprising a coding sequence for a protein of interest and a sequence comprising at least 80% sequence identity to a sequence selected from the group consisting of: SEQ ID Nos. 1-72. In some cases, the protein of interest is selected from the group consisting of: a transporter gene, an ion transporter gene, an exopolysaccharide biosynthesis gene, a cellulose biosynthesis gene, and a trehalose biosynthesis gene. In some cases, the protein of interest is selected from the group consisting of: a CysZ gene, a bcs gene, a treZ gene and an otsB gene. In some cases, the polynucleotide comprises a sequence with at least 85% sequence identity to a sequence selected from the group consisting of: SEQ ID Nos. 1-72. In some cases, the polynucleotide comprises a sequence with at least 90% sequence identity to a sequence selected from the group consisting of: SEQ ID Nos. 1-72. In some cases, the polynucleotide comprises a sequence with at least 95% sequence identity to a sequence selected from the group consisting of: SEQ ID Nos. 1-72. In some cases, the polynucleotide comprises a sequence with at least 97% sequence identity to a sequence selected from the group consisting of: SEQ ID Nos. 1-72. In some cases, the polynucleotide comprises a sequence with at least 98% sequence identity to a sequence selected from the group consisting of: SEQ ID Nos. 1-72. In some cases, the polynucleotide comprises a sequence with at least 99% sequence identity to a sequence selected from the group consisting of: SEQ ID Nos. 1-72.

In some embodiments, the present disclosure provides a method of increasing an amount of atmosphere derived nitrogen in a plant, comprising contacting said plant with a genetically engineered bacterium, wherein said genetically engineered bacterium comprises a nitrogen fixation coding sequence operably linked to a promoter comprising at least 80% sequence identity to a sequence selected from the group consisting of SEQ ID Nos. 1-72. In some cases, the promoter comprises at least 85% sequence identity to a sequence selected from the group consisting of SEQ ID Nos. 1-72. In some cases, the promoter comprises at least 90% sequence identity to a sequence selected from the group consisting of SEQ ID Nos. 1-72. In some cases, the promoter comprises at least 95% sequence identity to a sequence selected from the group consisting of SEQ ID Nos. 1-72. In some cases, the promoter comprises at least 97% sequence identity to a sequence selected from the group consisting of SEQ ID Nos. 1-72. In some cases, the promoter comprises at least 98% sequence identity to a sequence selected from the group consisting of SEQ ID Nos. 1-72. In some cases, increasing expression of said microbial gene increases ammonium excretion by said microbe. In some cases, increasing expression of said microbial gene increases nitrogen fixation by said microbe. In some cases, increasing expression of said microbial gene increases colonization of a plant by said microbe.

In some cases, the promoter comprises at least 99% sequence identity to a sequence selected from the group consisting of SEQ ID Nos. 1-72. In some cases, the genetically engineered bacterium is selected from the group consisting of Rahnella aquatilis, Klebsiella variicola, Achromobacter spiritinus, Achromobacter marplatensis, Microbacterium murale, Kluyvera intermedia, Kosakonia pseudosacchari, Enterobacter sp., Azospirillum lipoferum, and Kosakonia sacchari.

In some embodiments, the present disclosure provides method of decreasing an amount of nitrogen fertilizer required between planting and harvesting of a crop, the method comprising inoculating said crop with the genetically engineered bacterium described herein.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A-B depicts enrichment and isolation of nitrogen fixing bacteria. (A) Nfb agar plate was used to isolate single colonies of nitrogen fixing bacteria. (B) Semi-solid Nfb agar casted in Balch tube. The arrow points to pellicle of enriched nitrogen fixing bacteria.

FIG. 2 depicts a representative nifH PCR screen. Positive bands were observed at ˜350 bp for two colonies in this screen. Lower bands represent primer-dimers.

FIG. 3 depicts an example of a PCR screen of colonies from CRISPR-Cas-selected mutagenesis. CI006 colonies were screened with primers specific for the nifL locus. The wild type PCR product is expected at ˜2.2 kb, whereas the mutant is expected at ˜1.1 kb. Seven of ten colonies screened unambiguously show the desired deletion.

FIGS. 4A-D depict in vitro phenotypes of various strains. The Acetylene Reduction Assay (ARA) activities of mutants of strain CI010 (FIG. 4A) and mutants of strain CI006 (FIG. 4B) grown in nitrogen fixation media supplemented with 0 to 10 mM glutamine. ARA activities of additional strains are shown in FIG. 4C, and the ammonium excretion profile across time of two strains is shown in FIG. 4D.

FIG. 5 depicts in culture expression profile of 9 different genes in strains CI006 involved in diazaotrophic nitrogen fixation. Numbers represent counts of each transcript. Various conditions (0, 1, 10 mM Glutamine and 0%, 10%, 20% atmospheric air in N₂) are indicated.

FIG. 6 depicts CI006 colonization of corn roots. Corn seedlings were inoculated with CI006 harboring an RFP expression plasmid. After two weeks of growth and plasmid maintenance through watering with the appropriate antibiotic, roots were harvested and imaged through fluorescence microscopy. Colonization of the root intercellular space is observed.

FIG. 7 depicts nitrogen derived from microbe level in WT (C1050) and optimized (CM002) strain.

FIG. 8 shows an experimental setup for a Micro-Tom fruiting mass assay.

FIG. 9 shows a screen of 10 strains for increase in Micro-Tom plant fruit mass. Results for six replicates are presented. For column 3, p=0.07. For column 7, p=0.05.

FIGS. 10A-C depicts additional results for ARA activities of candidate microbes and counterpart candidate mutants grown in nitrogen fixation media supplemented with 0 to 10 mM glutamine.

FIG. 11 depicts a double mutant that exhibits higher ammonia excretion than the single mutant from which it was derived.

FIG. 12 depicts NDFA obtained from 15N Gas Uptake experiment (extrapolated back using days exposed) to measure NDFA in Corn plants in fertilized condition.

FIG. 13 depicts NDFA value obtained from 15N Gas Uptake experiment (extrapolated back using days exposed) to measure NDFA in Setaria plants in fertilized condition.

FIG. 14A depicts rate of incorporation of 15N gas. Plants inoculated with evolved strain showed increase in 15N gas incorporation compared to uninoculated plants.

FIG. 14B depicts 4 weeks after planting, up to 7% of the nitrogen in plants inoculated with an evolved strain is derived from microbially fixed nitrogen.

FIG. 14C depicts leaf area (and other biomass measurement, data not shown) is increased in plants inoculated with an evolved strain when compared to uninoculated or wild type inoculated plants.

FIG. 15A depicts evolved strains that show significantly higher nifH production in the root tissue, as measured by in planta transcriptomic study.

FIG. 15B depicts that rate of fixed nitrogen found in plant tissue is correlated with the rate in which that particular plant is colonized by HoME optimized strain.

FIG. 16A depicts a soil texture map of various field soils tested for colonization.

Soils in which a few microbes were originally source from are indicated as stars.

FIG. 16B depicts the colonization rate of Strain 1 and Strain 5 that are tested across four different soil types (circles). Both strains showed relatively robust colonization profile across diverse soil types.

FIG. 16C depicts colonization of Strain 1 as tested in a field trial over the span of a growing season. Strain 1 persists in the corn tissue up to week 12 after planting and starts to show decline in colonization after that time.

FIG. 17 depicts a schematic of microbe breeding, in accordance with embodiments.

FIG. 18 depicts an expanded view of the measurement of microbiome composition as shown in FIG. 17.

FIG. 19A illustrates several examples of promoter insertions showing an increase in nifA transcription in Kosakonia sacchari. NifA transcription was measured by qPCR, with 3 replicates, and using helD as a housekeeping gene for normalization of transcript counts, in accordance with some embodiments. Cells were cultured in minimal nitrogen free media.

FIG. 19B illustrates several examples of promoter insertions upstream of the nifA gene which lead to increased nitrogenase activity in Kosakonia sacchari. Activity was measured in an ARA assay in minimal media supplemented with 5 mM glutamine as the sole N source, in accordance with some embodiments.

FIG. 20A illustrates examples of promoter insertions showing an increase in nifA transcription in Klebsiella variicola. NifA transcription was measured by qPCR, using helD as a housekeeping gene for normalization of transcript counts, in accordance with some embodiments. Cells were cultured in minimal media supplemented with 10 mM glutamine as the sole N source.

FIG. 20B illustrates that the modifications of FIG. 20A lead to increased nitrogenase activity in Klebsiella variicola. Activity was measured in an ARA assay in minimal media supplemented with 10 mM glutamine as the sole N source, in accordance with some embodiments.

FIG. 21 and FIG. 22 illustrate further examples of promoter insertions showing an increase in nifA transcription in Klebsiella variicola. NifA transcription was measured by qPCR, using helD as a housekeeping gene for normalization of transcript counts, in accordance with some embodiments. Cells were cultured in minimal media supplemented with 10 mM glutamine as the sole N source.

FIG. 23 illustrates that the modifications of FIG. 22 lead to increased nitrogenase activity in Klebsiella variicola. Activity was measured in an ARA assay in minimal media supplemented with 10 mM glutamine as the sole N source, in accordance with some embodiments. Error bars represent standard deviation of the mean.

FIG. 24A and FIG. 24B illustrate examples of promoter insertions upstream of the nifA gene which lead to increased nitrogenase activity in Klebsiella variicola, in accordance with some embodiments. Scatter plots of two biological replicates are shown, measured in an ARA assay in minimal media supplemented with 5 mM ammonium phosphate.

FIG. 25A illustrates examples of promoter insertions upstream of the nifA gene which lead to increased nitrogenase activity in Rahnella aquatilis, in accordance with some embodiments. Activity was measured in an ARA assay in minimal media supplemented with 5 mM ammonium phosphate.

FIG. 25B illustrates an example of a promoter insertion upstream of the nifA gene which lead to increased nitrogenase activity in Rahnella aquatilis, in accordance with some embodiments. Activity was measured in an ARA assay in minimal media supplemented with 10 mM ammonium phosphate.

FIG. 26 illustrates examples of promoter insertions upstream of the nifA gene which lead to increased nitrogenase activity in Kosakonia pseudosacchari, in accordance with some embodiments. Activity was measured in an ARA assay in minimal media supplemented with 5 mM glutamine.

FIG. 27 illustrates an example of a promoter insertion upstream of the nifA gene which leads to increased nitrogenase activity in an Enterobacter species, in accordance with some embodiments. Activity was measured in an ARA assay in minimal media supplemented with 5 mM ammonium phosphate.

FIG. 28 illustrates examples of promoter insertions upstream of the nifA gene which lead to increased nitrogenase activity in a Klebsiella species, in accordance with some embodiments. Activity was measured in an ARA assay in minimal media supplemented with 5 mM ammonium phosphate.

FIG. 29 illustrates two examples of promoter insertions upstream of the nifA gene which lead to increased nitrogenase activity, in accordance with some embodiments. Activity was measured in an ARA assay in minimal media supplemented with 5 mM glutamine.

FIG. 30 illustrates an example of a promoter insertion showing an increase in bcsI transcription in Kosakonia sacchari. BcsI transcription was measured by qPCR, using helD as a housekeeping gene for normalization of transcript counts, in accordance with some embodiments. Cells were cultured in minimal nitrogen free media.

FIG. 31A illustrates an example of promoter insertions showing an increase in cysZ transcription in Kosakonia sacchari. CysZ transcription was measured by qPCR using helD as a housekeeping gene for normalization of transcript counts, in accordance with some embodiments. Cells were cultured in minimal media supplemented with 5 mM glutamine.

FIG. 31B illustrates an example of a promoter insertion showing an increase in otsB transcription in Kosakonia sacchari. OtsB transcription was measured by qPCR, using helD as a housekeeping gene for normalization of transcript counts, in accordance with some embodiments. Cells were cultured in minimal media supplemented with 5 mM glutamine.

FIG. 31C illustrates an example of a promoter insertion showing an increase in treZ transcription in Kosakonia sacchari. TreZ transcription was measured by qPCR, and using helD as a housekeeping gene for normalization of transcript counts, in accordance with some embodiments. Cells were cultured in minimal media supplemented with 5 mM glutamine.

FIG. 32A illustrates the promoter insertion method used in FIGS. 20A-23.

FIG. 32B illustrates the promoter insertion method used in FIGS. 19A, 19B, and 24A-31C.

### DETAILED DESCRIPTION OF THE INVENTION

The terms “polynucleotide”, “nucleotide”, “nucleotide sequence”, “nucleic acid” and “oligonucleotide” are used interchangeably. They refer to a polymeric form of nucleotides of any length, either deoxyribonucleotides or ribonucleotides, or analogs thereof.

Polynucleotides may have any three dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA (tRNA), ribosomal RNA (rRNA), short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, and primers. A polynucleotide may comprise one or more modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

“Hybridization” refers to a reaction in which one or more polynucleotides react to form a complex that is stabilized via hydrogen bonding between the bases of the nucleotide residues. The hydrogen bonding may occur by Watson Crick base pairing, Hoogstein binding, or in any other sequence specific manner according to base complementarity. The complex may comprise two strands forming a duplex structure, three or more strands forming a multi stranded complex, a single self-hybridizing strand, or any combination of these. A hybridization reaction may constitute a step in a more extensive process, such as the initiation of PCR, or the enzymatic cleavage of a polynucleotide by an endonuclease. A second sequence that is complementary to a first sequence is referred to as the “complement” of the first sequence. The term “hybridizable” as applied to a polynucleotide refers to the ability of the polynucleotide to form a complex that is stabilized via hydrogen bonding between the bases of the nucleotide residues in a hybridization reaction.

“Complementarity” refers to the ability of a nucleic acid to form hydrogen bond(s) with another nucleic acid sequence by either traditional Watson-Crick or other non-traditional types. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary, respectively). “Perfectly complementary” means that all the contiguous residues of a nucleic acid sequence will hydrogen bond with the same number of contiguous residues in a second nucleic acid sequence. “Substantially complementary” as used herein refers to a degree of complementarity that is at least 60%, 65%, 70%, 75%, 80%, 85%, 90%, 95%, 97%, 98%, 99%, or 100% over a region of 8, 9, 10, 11, 12, 13, 14, 15, 16, 17, 18, 19, 20, 21, 22, 23, 24, 25, 30, 35, 40, 45, 50, or more nucleotides, or refers to two nucleic acids that hybridize under stringent conditions. Sequence identity, such as for the purpose of assessing percent complementarity, may be measured by any suitable alignment algorithm, including but not limited to the Needleman-Wunsch algorithm (see e.g. the EMBOSS Needle aligner available at www.ebi.ac.uk/Tools/psa/emboss_needle/nucleotide.html, optionally with default settings), the BLAST algorithm (see e.g. the BLAST alignment tool available at blast.ncbi.nlm.nih.gov/Blast.cgi, optionally with default settings), or the Smith-Waterman algorithm (see e.g. the EMBOSS Water aligner available at www.ebi.ac.uk/Tools/psa/emboss_water/nucleotide.html, optionally with default settings). Optimal alignment may be assessed using any suitable parameters of a chosen algorithm, including default parameters.

In general, “stringent conditions” for hybridization refer to conditions under which a nucleic acid having complementarity to a target sequence predominantly hybridizes with a target sequence, and substantially does not hybridize to non-target sequences. Stringent conditions are generally sequence-dependent and vary depending on a number of factors. In general, the longer the sequence, the higher the temperature at which the sequence specifically hybridizes to its target sequence. Non-limiting examples of stringent conditions are described in detail in Tijssen (1993), Laboratory Techniques In Biochemistry And Molecular Biology-Hybridization With Nucleic Acid Probes Part I, Second Chapter “Overview of principles of hybridization and the strategy of nucleic acid probe assay”, Elsevier, N.Y.

In general, “sequence identiity” refers to an exact nucleotide-to-nucleotide or amino acid-to-amino acid correspondence of two polynucleotides or polypeptide sequences, respectively. Typically, techniques for determining sequence identity include determining the nucleotide sequence of a polynucleotide and/or determining the amino acid sequence encoded thereby, and comparing these sequences to a second nucleotide or amino acid sequence. Two or more sequences (polynucleotide or amino acid) can be compared by determining their “percent identity.” The percent identity of two sequences, whether nucleic acid or amino acid sequences, may be calculated as the number of exact matches between two aligned sequences divided by the length of the shorter sequences and multiplied by 100. In some cases, the percent identity of a test sequence and a reference sequence, whether nucleic acid or amino acid sequences, may be calculated as the number of exact matches between two aligned sequences divided by the length of the reference sequence and multiplied by 100. Percent identity may also be determined, for example, by comparing sequence information using the advanced BLAST computer program, including version 2.2.9, available from the National Institutes of Health. The BLAST program is based on the alignment method of Karlin and Altschul, Proc. Natl. Acad. Sci. USA 87:2264-2268 (1990) and as discussed in Altschul, et al., J. Mol. Biol. 215:403-410 (1990); Karlin And Altschul, Proc. Natl. Acad. Sci. USA 90:5873-5877 (1993); and Altschul et al., Nucleic Acids Res. 25:3389-3402 (1997). Briefly, the BLAST program defines identity as the number of identical aligned symbols (generally nucleotides or amino acids), divided by the total number of symbols in the shorter of the two sequences. The program may be used to determine percent identity over the entire length of the proteins being compared. Default parameters are provided to optimize searches with short query sequences in, for example, with the blastp program. The program also allows use of an SEG filter to mask-off segments of the query sequences as determined by the SEG program of Wootton and Federhen, Computers and Chemistry 17:149-163 (1993). Ranges of desired degrees of sequence identity are approximately 80% to 100% and integer values therebetween. Typically, the percent identities between a disclosed sequence and a claimed sequence are at least 80%, at least 85%, at least 90%, at least 95%, at least 98% or at least 99%.

As used herein, “expression” refers to the process by which a polynucleotide is transcribed from a DNA template (such as into and mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell.

The terms “polypeptide”, “peptide” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. As used herein the term “amino acid” includes natural and/or unnatural or synthetic amino acids, including glycine and both the D or L optical isomers, and amino acid analogs and peptidomimetics.

As used herein, the term “about” is used synonymously with the term “approximately.” Illustratively, the use of the term “about” with regard to an amount indicates that values slightly outside the cited values, e.g., plus or minus 0.1% to 10%.

The term “biologically pure culture” or “substantially pure culture” refers to a culture of a bacterial species described herein containing no other bacterial species in quantities sufficient to interfere with the replication of the culture or be detected by normal bacteriological techniques.

“Plant productivity” refers generally to any aspect of growth or development of a plant that is a reason for which the plant is grown. For food crops, such as grains or vegetables, “plant productivity” can refer to the yield of grain or fruit harvested from a particular crop. As used herein, improved plant productivity refers broadly to improvements in yield of grain, fruit, flowers, or other plant parts harvested for various purposes, improvements in growth of plant parts, including stems, leaves and roots, promotion of plant growth, maintenance of high chlorophyll content in leaves, increasing fruit or seed numbers, increasing fruit or seed unit weight, reducing NO₂ emission due to reduced nitrogen fertilizer usage and similar improvements of the growth and development of plants.

Microbes in and around food crops can influence the traits of those crops. Plant traits that may be influenced by microbes include: yield (e.g., grain production, biomass generation, fruit development, flower set); nutrition (e.g., nitrogen, phosphorus, potassium, iron, micronutrient acquisition); abiotic stress management (e.g., drought tolerance, salt tolerance, heat tolerance); and biotic stress management (e.g., pest, weeds, insects, fungi, and bacteria). Strategies for altering crop traits include: increasing key metabolite concentrations; changing temporal dynamics of microbe influence on key metabolites; linking microbial metabolite production/degradation to new environmental cues; reducing negative metabolites; and improving the balance of metabolites or underlying proteins.

As used herein, a “control sequence” refers to an operator, promoter, silencer, or terminator.

In some embodiments, native or endogenous control sequences of genes of the present disclosure are replaced with one or more intrageneric control sequences.

As used herein, “introduced” refers to the introduction by means of modern biotechnology, and not a naturally occurring introduction.

In some embodiments, the bacteria of the present disclosure have been modified such that they are not naturally occurring bacteria.

In some embodiments, the bacteria of the present disclosure are present in the plant in an amount of at least 10³ cfu, 10⁴ cfu, 10⁵ cfu, 10⁶ cfu, 10⁷ cfu, 10⁸ cfu, 10⁹ cfu, 10¹⁰ cfu, 10¹¹ cfu, or 10¹² cfu per gram of fresh or dry weight of the plant. In some embodiments, the bacteria of the present disclosure are present in the plant in an amount of at least about 10³ cfu, about 10⁴ cfu, about 10⁵ cfu, about 10⁶ cfu, about 10⁷ cfu, about 10⁸ cfu, about 10⁹ cfu, about 10¹⁰ cfu, about 10¹¹ cfu, or about 10¹² cfu per gram of fresh or dry weight of the plant. In some embodiments, the bacteria of the present disclosure are present in the plant in an amount of at least 10³ to 10⁹, 10³ to 10⁷, 10³ to 10⁵, 10⁵ to 10⁹, 10⁵ to 10⁷, 10⁶ to 10¹⁰, 10⁶ to 10⁷ cfu per gram of fresh or dry weight of the plant.

Fertilizers and exogenous nitrogen of the present disclosure may comprise the following nitrogen-containing molecules: ammonium, nitrate, nitrite, ammonia, glutamine, etc. Nitrogen sources of the present disclosure may include anhydrous ammonia, ammonia sulfate, urea, diammonium phosphate, urea-form, monoammonium phosphate, ammonium nitrate, nitrogen solutions, calcium nitrate, potassium nitrate, sodium nitrate, etc.

As used herein, “exogenous nitrogen” refers to non-atmospheric nitrogen readily available in the soil, field, or growth medium that is present under non-nitrogen limiting conditions, including ammonia, ammonium, nitrate, nitrite, urea, uric acid, ammonium acids, etc.

As used herein, “non-nitrogen limiting conditions” refers to non-atmospheric nitrogen available in the soil, field, media at concentrations greater than about 4 mM nitrogen, as disclosed by Kant et al. (2010. J. Exp. Biol. 62(4):1499-1509), which is incorporated herein by reference.

As used herein, “introduced genetic material” means genetic material that is added to, and remains as a component of, the genome of the recipient.

In some embodiments, the nitrogen fixation and assimilation genetic regulatory network comprises polynucleotides encoding genes and non-coding sequences that direct, modulate, and/or regulate microbial nitrogen fixation and/or assimilation and can comprise polynucleotide sequences of the nif cluster (e.g., nifA, nifB, nifC, . . . , nifZ), polynucleotides encoding nitrogen regulatory protein C, polynucleotides encoding nitrogen regulatory protein B, polynucleotide sequences of the gln cluster (e.g. glnA and glnD), draT, and ammonia transporters/permeases. In some cases, the Nif cluster may comprise NifB, NifH, NifD, NifK, NifE, NifN, NifX, hesa, and NifV. In some cases, the Nif cluster may comprise a subset of NifB, NifH, NifD, NifK, NifE, NifN, NifX, hesa, and NifV.

In some embodiments, fertilizer of the present disclosure comprises at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% nitrogen by weight.

In some embodiments, fertilizer of the present disclosure comprises at least about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% nitrogen by weight.

In some embodiments, fertilizer of the present disclosure comprises about 5% to 50%, about 5% to 75%, about 10% to 50%, about 10% to 75%, about 15% to 50%, about 15% to 75%, about 20% to 50%, about 20% to 75%, about 25% to 50%, about 25% to 75%, about 30% to 50%, about 30% to 75%, about 35% to 50%, about 35% to 75%, about 40% to 50%, about 40% to 75%, about 45% to 50%, about 45% to 75%, or about 50% to 75% nitrogen by weight.

In some embodiments, the increase of nitrogen fixation and/or the production of 1% or more of the nitrogen in the plant are measured relative to control plants, which have not been exposed to the bacteria of the present disclosure. All increases or decreases in bacteria are measured relative to control bacteria. All increases or decreases in plants are measured relative to control plants.

As used herein, a “constitutive promoter” is a promoter, which is active under most conditions and/or during most development stages. There are several advantages to using constitutive promoters in expression vectors used in biotechnology, such as: high level of production of proteins used to select transgenic cells or organisms; high level of expression of reporter proteins or scorable markers, allowing easy detection and quantification; high level of production of a transcription factor that is part of a regulatory transcription system; production of compounds that requires ubiquitous activity in the organism; and production of compounds that are required during all stages of development. Non-limiting exemplary constitutive promoters include, CaMV 35S promoter, opine promoters, ubiquitin promoter, alcohol dehydrogenase promoter, etc.

As used herein, a “non-constitutive promoter” is a promoter which is active under certain conditions, in certain types of cells, and/or during certain development stages. For example, tissue specific, tissue preferred, cell type specific, cell type preferred, inducible promoters, and promoters under development control are non-constitutive promoters. Examples of promoters under developmental control include promoters that preferentially initiate transcription in certain tissues.

As used herein, “inducible” or “repressible” promoter is a promoter which is under chemical or environmental factors control. Examples of environmental conditions that may affect transcription by inducible promoters include anaerobic conditions, certain chemicals, the presence of light, acidic or basic conditions, etc.

As used herein, a “tissue specific” promoter is a promoter that initiates transcription only in certain tissues. Unlike constitutive expression of genes, tissue-specific expression is the result of several interacting levels of gene regulation. As such, in the art sometimes it is preferable to use promoters from homologous or closely related species to achieve efficient and reliable expression of transgenes in particular tissues. This is one of the main reasons for the large amount of tissue-specific promoters isolated from particular tissues found in both scientific and patent literature.

As used herein, the term “operably linked” refers to the association of nucleic acid sequences on a single nucleic acid fragment so that the function of one is regulated by the other. For example, a promoter is operably linked with a coding sequence when it is capable of regulating the expression of that coding sequence (i.e., that the coding sequence is under the transcriptional control of the promoter). Coding sequences can be operably linked to regulatory sequences in a sense or antisense orientation. In another example, the complementary RNA regions of the disclosure can be operably linked, either directly or indirectly, 5′ to the target mRNA, or 3′ to the target mRNA, or within the target mRNA, or a first complementary region is 5′ and its complement is 3′ to the target mRNA.

### Regulation of Nitrogen Fixation

One trait that may be targeted for regulation by the methods described herein is nitrogen fixation. Nitrogen fertilizer is the largest operational expense on a farm and the biggest driver of higher yields in row crops like corn and wheat. Described herein are microbial products that can deliver renewable forms of nitrogen in non-leguminous crops. While some endophytes have the genetics necessary for fixing nitrogen in pure culture, the fundamental technical challenge is that wild-type endophytes of cereals and grasses stop fixing nitrogen in fertilized fields. The application of chemical fertilizers and residual nitrogen levels in field soils signal the microbe to shut down the biochemical pathway for nitrogen fixation.

Changes to the transcriptional and post-translational levels of nitrogen fixation regulatory network are required to develop a microbe capable of fixing and transferring nitrogen to corn in the presence of fertilizer. To that end, described herein is Host-Microbe Evolution (HoME) technology to precisely evolve regulatory networks and elicit novel phenotypes. Also described herein are unique, proprietary libraries of nitrogen-fixing endophytes isolated from corn, paired with extensive omics data surrounding the interaction of microbes and host plant under different environmental conditions like nitrogen stress and excess. This technology enables precision evolution of the genetic regulatory network of endophytes to produce microbes that actively fix nitrogen even in the presence of fertilizer in the field. Also described herein are evaluations of the technical potential of evolving microbes that colonize corn root tissues and produce nitrogen for fertilized plants and evaluations of the compatibility of endophytes with standard formulation practices and diverse soils to determine feasibility of integrating the microbes into modern nitrogen management strategies.

In order to utilize elemental nitrogen (N) for chemical synthesis, life forms combine nitrogen gas (N₂) available in the atmosphere with hydrogen in a process known as nitrogen fixation. Because of the energy-intensive nature of biological nitrogen fixation, diazotrophs (bacteria and archaea that fix atmospheric nitrogen gas) have evolved sophisticated and tight regulation of the nif gene cluster in response to environmental oxygen and available nitrogen. Nif genes encode enzymes involved in nitrogen fixation (such as the nitrogenase complex) and proteins that regulate nitrogen fixation. Shamseldin (2013. Global J. Biotechnol. Biochem. 8(4):84-94) discloses detailed descriptions of nif genes and their products, and is incorporated herein by reference. Described herein are methods of producing a plant with an improved trait comprising isolating bacteria from a first plant, introducing a genetic variation into a nif gene of the isolated bacteria, exposing a second plant to the variant bacteria, isolating bacteria from the second plant having an improved trait relative to the first plant, and repeating the steps with bacteria isolated from the second plant.

In Proteobacteria, regulation of nitrogen fixation centers around the σ₅₄-dependent enhancer-binding protein NifA, the positive transcriptional regulator of the nif cluster. Intracellular levels of active NifA are controlled by two key factors: transcription of the nifLA operon, and inhibition of NifA activity by protein-protein interaction with NifL. Both of these processes are responsive to intraceullar glutamine levels via the PII protein signaling cascade. This cascade is mediated by GlnD, which directly senses glutamine and catalyzes the uridylylation or deuridylylation of two PII regulatory proteins—GlnB and GlnK—in response the absence or presence, respectively, of bound glutamine. Under conditions of nitrogen excess, unmodified GlnB signals the deactivation of the nifLA promoter. However, under conditions of nitrogen limitation, GlnB is post-translationally modified, which inhibits its activity and leads to transcription of the nifLA operon. In this way, nifLA transcription is tightly controlled in response to environmental nitrogen via the PII protein signaling cascade. On the post-translational level of NifA regulation, GlnK inhibits the NifL/NifA interaction in a matter dependent on the overall level of free GlnK within the cell.

NifA is transcribed from the nifLA operon, whose promoter is activated by phosphorylated NtrC, another σ₅₄-dependent regulator. The phosphorylation state of NtrC is mediated by the histidine kinase NtrB, which interacts with deuridylylated GlnB but not uridylylated GlnB. Under conditions of nitrogen excess, a high intracellular level of glutamine leads to deuridylylation of GlnB, which then interacts with NtrB to deactivate its phosphorylation activity and activate its phosphatase activity, resulting in dephosphorylation of NtrC and the deactivation of the nifLA promoter. However, under conditions of nitrogen limitation, a low level of intracellular glutamine results in uridylylation of GlnB, which inhibits its interaction with NtrB and allows the phosphorylation of NtrC and transcription of the nifLA operon. In this way, nifLA expression is tightly controlled in response to environmental nitrogen via the PII protein signaling cascade. nifA, ntrB, ntrC, and glnB, are all genes that can be mutated in the methods described herein. These processes may also be responsive to intracellular, or extracellular, levels of ammonia, urea or nitrates.

The activity of NifA is also regulated post-translationally in response to environmental nitrogen, most typically through NifL-mediated inhibition of NifA activity. In general, the interaction of NifL and NifA is influenced by the PII protein signaling cascade via GlnK, although the nature of the interactions between GlnK and NifL/NifA varies significantly between diazotrophs. In Klebsiella pneumoniae, both forms of GlnK inhibit the NifL/NifA interaction, and the interaction between GlnK and NifL/NifA is determined by the overall level of free GlnK within the cell. Under nitrogen-excess conditions, deuridylylated GlnK interacts with the ammonium transporter AmtB, which serves to both block ammonium uptake by AmtB and sequester GlnK to the membrane, allowing inhibition of NifA by NifL. On the other hand, in Azotobacter vinelandii, interaction with deuridylylated GlnK is required for the NifL/NifA interaction and NifA inhibition, while uridylylation of GlnK inhibits its interaction with NifL. In diazotrophs lacking the nifL gene, there is evidence that NifA activity is inhibited directly by interaction with the deuridylylated forms of both GlnK and GlnB under nitrogen-excess conditions. In some bacteria the Nif cluster may be regulated by glnR, and further in some cases this may comprise negative regulation. Regardless of the mechanism, post-translational inhibition of NifA is an important regulator of the nif cluster in most known diazotrophs. Additionally, nifL, amtB, glnK, and glnR are genes that can be mutated in the methods described herein.

In addition to regulating the transcription of the nif gene cluster, many diazotrophs have evolved a mechanism for the direct post-translational modification and inhibition of the nitrogenase enzyme itself, known as nitrogenase shutoff. This is mediated by ADP-ribosylation of the Fe protein (NifH) under nitrogen-excess conditions, which disrupts its interaction with the MoFe protein complex (NifDK) and abolishes nitrogenase activity. DraT catalyzes the ADP-ribosylation of the Fe protein and shutoff of nitrogenase, while DraG catalyzes the removal of ADP-ribose and reactivation of nitrogenase. As with nifLA transcription and NifA inhibition, nitrogenase shutoff is also regulated via the PII protein signaling cascade. Under nitrogen-excess conditions, deuridylylated GlnB interacts with and activates DraT, while deuridylylated GlnK interacts with both DraG and AmtB to form a complex, sequestering DraG to the membrane. Under nitrogen-limiting conditions, the uridylylated forms of GlnB and GlnK do not interact with DraT and DraG, respectively, leading to the inactivation of DraT and the diffusion of DraG to the Fe protein, where it removes the ADP-ribose and activates nitrogenase. The methods described herein also contemplate introducing genetic variation into the nifH, nifD, nifK, and draT genes.

Although some endophytes have the ability to fix nitrogen in vitro, often the genetics are silenced in the field by high levels of exogenous chemical fertilizers. One can decouple the sensing of exogenous nitrogen from expression of the nitrogenase enzyme to facilitate field-based nitrogen fixation. Improving the integral of nitrogenase activity across time further serves to augment the production of nitrogen for utilization by the crop. Specific targets for genetic variation to facilitate field-based nitrogen fixation using the methods described herein include one or more genes selected from the group consisting of nifA, nifL, ntrB, ntrC, glnA, glnB, glnK, draT, amtB, glnD, glnE, nifJ, nifH, nifD, nifK, nifY nifE, nifN nifU, nifS, nifV, nifW, nifZ, nifM, nifF, nifB, and nifQ.

An additional target for genetic variation to facilitate field-based nitrogen fixation using the methods described herein is the NifA protein. The NifA protein is typically the activator for expression of nitrogen fixation genes. Increasing the production of NifA (either constitutively or during high ammonia condition) circumvents the native ammonia-sensing pathway. In addition, reducing the production of NifL proteins, a known inhibitor of NifA, also leads to an increased level of freely active NifA. In addition, increasing the transcription level of the nifAL operon (either constitutively or during high ammonia condition) also leads to an overall higher level of NifA proteins. Elevated level of nifAL expression is achieved by altering the promoter itself or by reducing the expression of NtrB (part of ntrB and ntrC signaling cascade that originally would result in the shutoff of nifAL operon during high nitrogen condition). High level of NifA achieved by these or any other methods described herein increases the nitrogen fixation activity of the endophytes.

Another target for genetic variation to facilitate field-based nitrogen fixation using the methods described herein is the GlnD/GlnB/GlnK PII signaling cascade. The intracellular glutamine level is sensed through the GlnD/GlnB/GlnK PII signaling cascade. Active site mutations in GlnD that abolish the uridylyl-removing activity of GlnD disrupt the nitrogen-sensing cascade. In addition, reduction of the GlnB concentration short circuits the glutamine-sensing cascade. These mutations “trick” the cells into perceiving a nitrogen-limited state, thereby increasing the nitrogen fixation level activity. These processes may also be responsive to intracellular, or extracellular, levels of ammonia, urea or nitrates.

The amtB protein is also a target for genetic variation to facilitate field-based nitrogen fixation using the methods described herein. Ammonia uptake from the environment can be reduced by decreasing the expression level of amtB protein. Without intracellular ammonia, the endophyte is not able to sense the high level of ammonia, preventing the down-regulation of nitrogen fixation genes. Any ammonia that manages to get into the intracellular compartment is converted into glutamine. Intracellular glutamine level is the major currency of nitrogen sensing. Decreasing the intracellular glutamine level prevents the cells from sensing high ammonium levels in the environment. This effect can be achieved by increasing the expression level of glutaminase, an enzyme that converts glutamine into glutamate. In addition, intracellular glutamine can also be reduced by decreasing glutamine synthase (an enzyme that converts ammonia into glutamine). In diazotrophs, fixed ammonia is quickly assimilated into glutamine and glutamate to be used for cellular processes. Disruptions to ammonia assimilation may enable diversion of fixed nitrogen to be exported from the cell as ammonia. The fixed ammonia is predominantly assimilated into glutamine by glutamine synthetase (GS), encoded by glnA, and subsequently into glutamine by glutamine oxoglutarate aminotransferase (GOGAT). In some examples, glnS encodes a glutamine synthetase. GS is regulated post-translationally by GS adenylyl transferase (GlnE), a bi-functional enzyme encoded by glnE that catalyzes both the adenylylation and de-adenylylation of GS through activity of its adenylyl-transferase (AT) and adenylyl-removing (AR) domains, respectively. Under nitrogen limiting conditions, glnA is expressed, and GlnE's AR domain de-adynylylates GS, allowing it to be active. Under conditions of nitrogen excess, glnA expression is turned off, and GlnE's AT domain is activated allosterically by glutamine, causing the adenylylation and deactivation of GS.

Furthermore, the draT gene may also be a target for genetic variation to facilitate field-based nitrogen fixation using the methods described herein. Once nitrogen fixing enzymes are produced by the cell, nitrogenase shut-off represents another level in which cell downregulates fixation activity in high nitrogen condition. This shut-off could be removed by decreasing the expression level of DraT.

Methods for imparting new microbial phenotypes can be performed at the transcriptional, translational, and post-translational levels. The transcriptional level includes changes at the promoter (such as changing sigma factor affinity or binding sites for transcription factors, including deletion of all or a portion of the promoter) or changing transcription terminators and attenuators. The translational level includes changes at the ribosome binding sites and changing mRNA degradation signals. The post-translational level includes mutating an enzyme's active site and changing protein-protein interactions. These changes can be achieved in a multitude of ways. Reduction of expression level (or complete abolishment) can be achieved by swapping the native ribosome binding site (RBS) or promoter with another with lower strength/efficiency. ATG start sites can be swapped to a GTG, TTG, or CTG start codon, which results in reduction in translational activity of the coding region. Complete abolishment of expression can be done by knocking out (deleting) the coding region of a gene. Frameshifting the open reading frame (ORF) likely will result in a premature stop codon along the ORF, thereby creating a non-functional truncated product. Insertion of in-frame stop codons will also similarly create a non-functional truncated product. Addition of a degradation tag at the N or C terminal can also be done to reduce the effective concentration of a particular gene.

Conversely, expression level of the genes described herein can be achieved by using a stronger promoter. To ensure high promoter activity during high nitrogen level condition (or any other condition), a transcription profile of the whole genome in a high nitrogen level condition could be obtained and active promoters with a desired transcription level can be chosen from that dataset to replace the weak promoter. Weak start codons can be swapped out with an ATG start codon for better translation initiation efficiency. Weak ribosomal binding sites (RBS) can also be swapped out with a different RBS with higher translation initiation efficiency. In addition, site specific mutagenesis can also be performed to alter the activity of an enzyme.

Examples of promoters which may be used to drive expression of a gene as described herein, include the promoters in Table 8. Further details about these sequences are provided in Table 9. Table 9 lists the species each sequence was derived from, as well as the native gene, and native gene function of several of the sequences. In addition some of the sequences have been validated as promoters either in an in vitro transcription assay, an in planta transcription assay or in both, see Example 8.

Increasing the level of nitrogen fixation that occurs in a plant can lead to a reduction in the amount of chemical fertilizer needed for crop production and reduce greenhouse gas emissions (e.g., nitrous oxide).

### Generation of Bacterial Populations

### Isolation of Bacteria

Microbes useful in methods and compositions disclosed herein can be obtained by extracting microbes from surfaces or tissues of native plants. Microbes can be obtained by grinding seeds to isolate microbes. Microbes can be obtained by planting seeds in diverse soil samples and recovering microbes from tissues. Additionally, microbes can be obtained by inoculating plants with exogenous microbes and determining which microbes appear in plant tissues. Non-limiting examples of plant tissues may include a seed, seedling, leaf, cutting, plant, bulb, or tuber.

A method of obtaining microbes may be through the isolation of bacteria from soils. Bacteria may be collected from various soil types. In some example, the soil can be characterized by traits such as high or low fertility, levels of moisture, levels of minerals, and various cropping practices. For example, the soil may be involved in a crop rotation where different crops are planted in the same soil in successive planting seasons. The sequential growth of different crops on the same soil may prevent disproportionate depletion of certain minerals. The bacteria can be isolated from the plants growing in the selected soils. The seedling plants can be harvested at 2-6 weeks of growth. For example, at least 400 isolates can be collected in a round of harvest. Soil and plant types reveal the plant phenotype as well as the conditions, which allow for the downstream enrichment of certain phenotypes.

Microbes can be isolated from plant tissues to assess microbial traits. The parameters for processing tissue samples may be varied to isolate different types of associative microbes, such as rhizopheric bacteria, epiphytes, or endophytes. The isolates can be cultured in nitrogen-free media to enrich for bacteria that perform nitrogen fixation. Alternatively, microbes can be obtained from global strain banks.

In planta analytics are performed to assess microbial traits. In some embodiments, the plant tissue can be processed for screening by high throughput processing for DNA and RNA. Additionally, non-invasive measurements can be used to assess plant characteristics, such as colonization. Measurements on wild microbes can be obtained on a plant-by-plant basis. Measurements on wild microbes can also be obtained in the field using medium throughput methods. Measurements can be done successively over time. Model plant system can be used including, but not limited to, Setaria.

Microbes in a plant system can be screened via transcriptional profiling of a microbe in a plant system. Examples of screening through transcriptional profiling are using methods of quantitative polymerase chain reaction (qPCR), molecular barcodes for transcript detection, Next Generation Sequencing, and microbe tagging with fluorescent markers. Impact factors can be measured to assess colonization in the greenhouse including, but not limited to, microbiome, abiotic factors, soil conditions, oxygen, moisture, temperature, inoculum conditions, and root localization. Nitrogen fixation can be assessed in bacteria by measuring 15N gas/fertilizer (dilution) with IRMS or NanoSIMS as described herein. NanoSIMS is high-resolution secondary ion mass spectrometry technique. The NanoSIMS technique is a way to investigate chemical activity from biological samples. The catalysis of reduction of oxidation reactions that drive the metabolism of microorganisms can be investigated at the cellular, subcellular, molecular and elemental level. NanoSIMS can provide high spatial resolution of greater than 0.1 μm. NanoSIMS can detect the use of isotope tracers such as ¹³C, ¹⁵N, and ¹⁸O. Therefore, NanoSIMS can be used to measure the activity of nitrogen fixation in the cell.

Automated greenhouses can be used for planta analytics. Plant metrics in response to microbial exposure include, but are not limited to, biomass, chloroplast analysis, CCD camera, volumetric tomography measurements.

One way of enriching a microbe population is according to genotype. For example, a polymerase chain reaction (PCR) assay with a targeted primer or specific primer. Primers designed for the nifH gene can be used to identify diazotrophs because diazotrophs express the nifH gene in the process of nitrogen fixation. A microbial population can also be enriched via single-cell culture-independent approaches and chemotaxis-guided isolation approaches. Alternatively, targeted isolation of microbes can be performed by culturing the microbes on selection media. Premeditated approaches to enriching microbial populations for desired traits can be guided by bioinformatics data and are described herein.

Enriching for Microbes with Nitrogen Fixation Capabilities Using Bioinformatics

Bioinformatic tools can be used to identify and isolate plant growth promoting Rhizobacteria (PGPRs), which are selected based on their ability to perform nitrogen fixation. Microbes with high nitrogen fixing ability can promote favorable traits in plants. Bioinformatic modes of analysis for the identification of PGPRs include, but are not limited to, genomics, metagenomics, targeted isolation, gene sequencing, transcriptome sequencing, and modeling.

Genomics analysis can be used to identify PGPRs and confirm the presence of mutations with methods of Next Generation Sequencing as described herein and microbe version control.

Metagenomics can be used to identify and isolate PGPR using a prediction algorithm for colonization. Metadata can also be used to identify the presence of an engineered strain in environmental and greenhouse samples.

Transcriptomic sequencing can be used to predict genotypes leading to PGPR phenotypes. Additionally, transcriptomic data is used to identify promoters for altering gene expression. Transcriptomic data can be analyzed in conjunction with the Whole Genome Sequence (WGS) to generate models of metabolism and gene regulatory networks.

### Domestication of Microbes

Microbes isolated from nature can undergo a domestication process wherein the microbes are converted to a form that is genetically trackable and identifiable. One way to domesticate a microbe is to engineer it with antibiotic resistance. The process of engineering antibiotic resistance can begin by determining the antibiotic sensitivity in the wild type microbial strain. If the bacteria are sensitive to the antibiotic, then the antibiotic can be a good candidate for antibiotic resistance engineering. Subsequently, an antibiotic resistant gene or a counterselectable suicide vector can be incorporated into the genome of a microbe using recombineering methods. A counterselectable suicide vector may consist of a deletion of the gene of interest, a selectable marker, and the counterselectable marker sacB. Counterselection can be used to exchange native microbial DNA sequences with antibiotic resistant genes. A medium throughput method can be used to evaluate multiple microbes simultaneously allowing for parallel domestication. Alternative methods of domestication include the use of homing nucleases to prevent the suicide vector sequences from looping out or from obtaining intervening vector sequences.

DNA vectors can be introduced into bacteria via several methods including electroporation and chemical transformations. A standard library of vectors can be used for transformations. An example of a method of gene editing is CRISPR preceded by Cas9 testing to ensure activity of Cas9 in the microbes.

### Non-transgenic Engineering of Microbes

A microbial population with favorable traits can be obtained via directed evolution. Direct evolution is an approach wherein the process of natural selection is mimicked to evolve proteins or nucleic acids towards a user-defined goal. An example of direct evolution is when random mutations are introduced into a microbial population, the microbes with the most favorable traits are selected, and the growth of the selected microbes is continued. The most favorable traits in growth promoting Rhizobacteria (PGPRs) may be in nitrogen fixation. The method of directed evolution may be iterative and adaptive based on the selection process after each iteration.

Plant growth promoting Rhizobacteria (PGPRs) with high capability of nitrogen fixation can be generated. The evolution of PGPRs can be carried out via the introduction of genetic variation. Genetic variation can be introduced via polymerase chain reaction mutagenesis, oligonucleotide-directed mutagenesis, saturation mutagenesis, fragment shuffling mutagenesis, homologous recombination, CRISPR/Cas9 systems, chemical mutagenesis, and combinations thereof. These approaches can introduce random mutations into the microbial population. For example, mutants can be generated using synthetic DNA or RNA via oligonucleotide-directed mutagenesis. Mutants can be generated using tools contained on plasmids, which are later cured.

In some cases, mutants, or heterologous sequences endogenous to the host cell may be introduced without introducing any sequences exogenous to the host cell. In some cases, heterologous sequences from a cell of the same species as the host cell may be introduced without introducing any sequences exogenous to species of the host cell. Any plasmids used to construct such a cell may then be cured to produce a genetically engineered cell which contains to genetic material exogenous to the cell. In some cases, heterologous sequences from a cell of the same genus as the host cell may be introduced without introducing any sequences exogenous to genus of the host cell. Any plasmids used to construct such a cell may then be cured to produce a genetically engineered cell which contains to genetic material exogenous to the genus of the host cell, thus creating a non-intergeneric cell.

Genes of interest can be identified using libraries from other species with improved traits including, but not limited to, improved PGPR properties, improved colonization of cereals, increased oxygen sensitivity, increased nitrogen fixation, and increased ammonium excretion. Intrageneric genes can be designed based on these libraries using software such as Geneious or Platypus design software. Mutations can be designed with the aid of machine learning. Mutations can be designed with the aid of a metabolic model. Automated design of the mutation can be done using a la Platypus and will guide RNAs for Cas-directed mutagenesis.

The intra-generic genes can be transferred into the host microbe. Additionally, reporter systems can also be transferred to the microbe. The reporter systems characterize promoters, determine the transformation success, screen mutants, and act as negative screening tools.

The microbes carrying the mutation can be cultured via serial passaging. A microbial colony contains a single variant of the microbe. Microbial colonies are screened with the aid of an automated colony picker and liquid handler. Mutants with gene duplication and increased copy number express a higher genotype of the desired trait.

### Selection of Plant Growth Promoting Microbess Based on Nitrogen Fixation

The microbial colonies can be screened using various assays to assess nitrogen fixation. One way to measure nitrogen fixation is via a single fermentative assay, which measures nitrogen excretion. An alternative method is the acetylene reduction assay (ARA) with in-line sampling over time. ARA can be performed in high throughput plates of microtube arrays. ARA can be performed with live plants and plant tissues. The media formulation and media oxygen concentration can be varied in ARA assays. Another method of screening microbial variants is by using biosensors. The use of NanoSIMS and Raman microspectroscopy can be used to investigate the activity of the microbes. In some cases, bacteria can also be cultured and expanded using methods of fermentation in bioreactors. The bioreactors are designed to improve robustness of bacteria growth and to decrease the sensitivity of bacteria to oxygen. Medium to high TP plate-based microfermentors are used to evaluate oxygen sensitivity, nutritional needs, nitrogen fixation, and nitrogen excretion. The bacteria can also be co-cultured with competitive or beneficial microbes to elucidate cryptic pathways. Flow cytometry can be used to screen for bacteria that produce high levels of nitrogen using chemical, colorimetric, or fluorescent indicators. The bacteria may be cultured in the presence or absence of a nitrogen source. For example, the bacteria may be cultured with glutamine, ammonia, urea or nitrates.

### Microbe Breeding

Microbe breeding is a method to systematically identify and improve the role of species within the crop microbiome. The method comprises three steps: 1) selection of candidate species by mapping plant-microbe interactions and predicting regulatory networks linked to a particular phenotype, 2) pragmatic and predictable improvement of microbial phenotypes through intra-species crossing of regulatory networks and gene clusters, and 3) screening and selection of new microbial genotypes that produce desired crop phenotypes. To systematically assess the improvement of strains, a model is created that links colonization dynamics of the microbial community to genetic activity by key species. The model is used to predict genetic targets breeding and improve the frequency of selecting improvements in microbiome-encoded traits of agronomic relevance.

Production of bacteria to improve plant traits (e.g., nitrogen fixation) can be achieved through serial passage. The production of this bacteria can be done by selecting plants, which have a particular improved trait that is influenced by the microbial flora, in addition to identifying bacteria and/or compositions that are capable of imparting one or more improved traits to one or more plants. One method of producing a bacteria to improve a plant trait includes the steps of: (a) isolating bacteria from tissue or soil of a first plant; (b) introducing a genetic variation into one or more of the bacteria to produce one or more variant bacteria; (c) exposing a plurality of plants to the variant bacteria; (d) isolating bacteria from tissue or soil of one of the plurality of plants, wherein the plant from which the bacteria is isolated has an improved trait relative to other plants in the plurality of plants; and (e) repeating steps (b) to (d) with bacteria isolated from the plant with an improved trait (step (d)). Steps (b) to (d) can be repeated any number of times (e.g., once, twice, three times, four times, five times, ten times, or more) until the improved trait in a plant reaches a desired level. Further, the plurality of plants can be more than two plants, such as 10 to 20 plants, or 20 or more, 50 or more, 100 or more, 300 or more, 500 or more, or 1000 or more plants.

In addition to obtaining a plant with an improved trait, a bacterial population comprising bacteria comprising one or more genetic variations introduced into one or more genes (e.g., genes regulating nitrogen fixation) is obtained. By repeating the steps described above, a population of bacteria can be obtained that include the most appropriate members of the population that correlate with a plant trait of interest. The bacteria in this population can be identified and their beneficial properties determined, such as by genetic and/or phenotypic analysis. Genetic analysis may occur of isolated bacteria in step (a). Phenotypic and/or genotypic information may be obtained using techniques including: high through-put screening of chemical components of plant origin, sequencing techniques including high throughput sequencing of genetic material, differential display techniques (including DDRT-PCR, and DD-PCR), nucleic acid microarray techniques, RNA-sequencing (Whole Transcriptome Shotgun Sequencing), and qRT-PCR (quantitative real time PCR).

Information gained can be used to obtain community profiling information on the identity and activity of bacteria present, such as phylogenetic analysis or microarray-based screening of nucleic acids coding for components of rRNA operons or other taxonomically informative loci. Examples of taxonomically informative loci include 16S rRNA gene, 23S rRNA gene, 5S rRNA gene, 5.8S rRNA gene, 12S rRNA gene, 18S rRNA gene, 28S rRNA gene, gyrB gene, rpoB gene, fusA gene, recA gene, coxl gene, nifD gene. Example processes of taxonomic profiling to determine taxa present in a population are described in US20140155283. Bacterial identification may comprise characterizing activity of one or more genes or one or more signaling pathways, such as genes associated with the nitrogen fixation pathway. Synergistic interactions (where two components, by virtue of their combination, increase a desired effect by more than an additive amount) between different bacterial species may also be present in the bacterial populations.

The genetic variation may be a gene selected from the group consisting of: nifA, nifL, ntrB, ntrC, glnA, glnB, glnK, draT, amtB, glnD, glnE, nifJ, nifH, nifD, nifK, nifY, nifE, nifN, nifJ, nifS, nifV, nifW, nifZ, nifM, nifF, nifB, and nifQ. The genetic variation may be a variation in a gene encoding a protein with functionality selected from the group consisting of: glutamine synthetase, glutaminase, glutamine synthetase adenylyltransferase, transcriptional activator, anti-transcriptional activator, pyruvate flavodoxin oxidoreductase, flavodoxin, or NAD+-dinitrogen-reductase aDP-D-ribosyltransferase. The genetic variation may be a mutation that results in one or more of increased expression or activity of NifA or glutaminase; decreased expression or activity of NifL, NtrB, glutamine synthetase, GlnB, GlnK, DraT, AmtB; decreased adenylyl-removing activity of GlnE; or decreased uridylyl-removing activity of GlnD. Introducing a genetic variation may comprise insertion and/or deletion of one or more nucleotides at a target site, such as 1, 2, 3, 4, 5, 10, 25, 50, 100, 250, 500, or more nucleotides. The genetic variation introduced into one or more bacteria of the methods disclosed herein may be a knock-out mutation (e.g. deletion of a promoter, insertion or deletion to produce a premature stop codon, deletion of an entire gene), or it may be elimination or abolishment of activity of a protein domain (e.g. point mutation affecting an active site, or deletion of a portion of a gene encoding the relevant portion of the protein product), or it may alter or abolish a regulatory sequence of a target gene. One or more regulatory sequences may also be inserted, including heterologous regulatory sequences and regulatory sequences found within a genome of a bacterial species or genus corresponding to the bacteria into which the genetic variation is introduced. Moreover, regulatory sequences may be selected based on the expression level of a gene in a bacterial culture or within a plant tissue. In some cases, a regulatory sequence inserted may be selected from SEQ ID NOs.: 1-72. The genetic variation may be a pre-determined genetic variation that is specifically introduced to a target site. The genetic variation may be a random mutation within the target site. The genetic variation may be an insertion or deletion of one or more nucleotides. In some cases, a plurality of different genetic variations (e.g. 2, 3, 4, 5, 10, or more) are introduced into one or more of the isolated bacteria before exposing the bacteria to plants for assessing trait improvement. The plurality of genetic variations can be any of the above types, the same or different types, and in any combination. In some cases, a plurality of different genetic variations are introduced serially, introducing a first genetic variation after a first isolation step, a second genetic variation after a second isolation step, and so forth so as to accumulate a plurality of genetic variations in bacteria imparting progressively improved traits on the associated plants.

In some embodiments, a microbe may be genetically altered by introducing a regulatory sequence. For example, a sequence selected from SEQ ID NOs.: 1-72, or a sequence comprising a fragment of one of SEQ ID NOs.: 1-72, or a sequence which has at least about 60%, 70%, 80%, 85%, 90%, 95%, 97%, 98%, or 99% sequence identity to a sequence selected from SEQ ID NOs.: 1-72. In some cases, a regulatory sequence may comprise a sequence which has at least about 60%, 70%, 80%, 85%, 90%, 95%, 97%, 98%, or 99% sequence identity to a fragment of a sequence selected from SEQ ID NOs.: 1-72.

In some cases, a genetically engineered microbe, as described herein, may comprise an inserted sequence. In some cases, said inserted sequence may comprise a native sequence inserted in a non-native context. For example said inserted sequence may comprise a promoter from a first gene of said genetically engineered microbe inserted in the location of a promoter of a second gene of said microbe. In some cases, said inserted sequence may comprise a non-native promoter inserted in a non-native context. In some cases, said inserted sequence may comprise a non-native promoter inserted in a position equivalent to its native context. For example, said microbe of a first species may comprise a gene operably linked to the native promoter of said gene in a second species.

In some cases, a regulatory sequence may be inserted upstream of a gene so as to be operably linked to said gene. In some cases, inserting a regulatory sequence upstream of a gene may also involve deleting a native regulatory sequence of said gene. In some cases, a regulatory sequence may be inserted upstream of a Nif cluster gene so as to control said Nif cluster gene. In some cases, a regulatory sequence may be inserted upstream of a transporter gene so as to control said transporter gene. In some cases, a regulatory sequence may be inserted upstream of an ion transporter gene so as to control said ion transporter gene. In some cases, a regulatory sequence may be inserted upstream of a CysZ gene so as to control said CysZ gene. In some cases, a regulatory sequence may be inserted upstream of an exopolysaccharide biosynthesis gene so as to control said exopolysaccharide biosynthesis gene. In some cases, a regulatory sequence may be inserted upstream of a cellulose biosynthesis gene so as to control said cellulose biosynthesis gene. In some cases, a regulatory sequence may be inserted upstream of a bcs gene so as to control said bcs gene. In some cases, a regulatory sequence may be inserted upstream of a trehalose biosynthesis gene so as to control said trehalose biosynthesis gene. In some cases, a regulatory sequence may be inserted upstream of a treZ gene so as to control said treZ gene. In some cases, a regulatory sequence may be inserted so as to increase expression of a gene with a desired phenotype. Desired phenotypes may include nitrogen fixation, ammonium excretion, transport of components required for nitrogenase enzyme cofactors, biofilm formation, plant colonization, fitness or competitiveness in the rhizosphere, reactive oxygen species scavenging, expression of plant cell wall degrading enzymes, and root attachment.

In some cases, a regulatory sequence may be selected such that the genus of the microbe of origin of the regulatory sequence is the same as the genus of the microbe into which it is inserted. In some cases, a regulatory sequence may be selected such that the species of origin of the regulatory sequence is the same as the species into which it is inserted. For example, a regulatory element identified in a first Kosakonia sacchari bacterium is inserted into a new location in a second Kosakonia sacchari bacterium, thus creating a genetically modified Kosakonia sacchari bacterium which does not contain an intergeneric DNA sequence. In another example, a regulatory element identified in a Kosakonia pseudosacchari bacterium is inserted into a Kosakonia sacchari bacterium, thus creating a genetically modified Kosakonia sacchari bacterium which does not contain an intergeneric DNA sequence. In some cases, a Rahnella aquatilis bacterium is modified using a sequence selected from the group consisting of: SEQ ID Nos. 41-59, and 63-66. In some cases, a Kosakonia sacchari bacterium is modified using a sequence selected from the group consisting of: SEQ ID Nos. 1-10. In some cases, a Kosakonia sacchari bacterium is modified using a sequence selected from the group consisting of: SEQ ID Nos. 61, and 70-72. In some cases, a Klebsiella variicola bacterium is modified using a sequence selected from the group consisting of: SEQ ID Nos. 11-40. In some cases, a Kluyvera intermedia bacterium is modified using SEQ ID NO. 60. In some cases, a Kosakonia pseudosacchari bacterium is modified using a sequence selected from the group consisting of: SEQ ID Nos. 1-10. In some cases, a Kosakonia pseudosacchari bacterium is modified using a sequence selected from the group consisting of: SEQ ID Nos. 61, and 70-72. In some cases, an Enterobacter species bacterium is modified using SEQ ID NO. 62. In some cases, a Klebsiella species bacterium is modified using a sequence selected from the group consisting of: SEQ ID Nos. 67-69. In some cases, a regulatory sequence may be selected such that the species of origin of the regulatory sequence is the same as the species into which it is inserted, however the regulatory sequence may comprise one of more mutations.

In some cases, a regulatory sequence may be selected such that the species of origin of the regulatory sequence is not the same as the species into which it is inserted. For example, a regulatory element identified in a Kosakonia sacchari bacterium is inserted into a Klebsiella variicola bacterium, thus creating a genetically modified Klebsiella variicola bacterium which contains a intergeneric DNA sequence.

In general, the term “genetic variation” refers to any change introduced into a polynucleotide sequence relative to a reference polynucleotide, such as a reference genome or portion thereof, or reference gene or portion thereof. A genetic variation may be referred to as a “mutation,” and a sequence or organism comprising a genetic variation may be referred to as a “genetic variant” or “mutant”. Genetic variations can have any number of effects, such as the increase or decrease of some biological activity, including gene expression, metabolism, and cell signaling. Genetic variations can be specifically introduced to a target site, or introduced randomly. A variety of molecular tools and methods are available for introducing genetic variation. For example, genetic variation can be introduced via polymerase chain reaction mutagenesis, oligonucleotide-directed mutagenesis, saturation mutagenesis, fragment shuffling mutagenesis, homologous recombination, recombineering, lambda red mediated recombination, CRISPR/Cas9 systems, chemical mutagenesis, and combinations thereof. Chemical methods of introducing genetic variation include exposure of DNA to a chemical mutagen, e.g., ethyl methanesulfonate (EMS), methyl methanesulfonate (MMS), N-nitrosourea (EN U), N-methyl-N-nitro-N′-nitrosoguanidine, 4-nitroquinoline N-oxide, diethylsulfate, benzopyrene, cyclophosphamide, bleomycin, triethylmelamine, acrylamide monomer, nitrogen mustard, vincristine, diepoxyalkanes (for example, diepoxybutane), ICR-170, formaldehyde, procarbazine hydrochloride, ethylene oxide, dimethylnitrosamine, 7,12 dimethylbenz(a)anthracene, chlorambucil, hexamethylphosphoramide, bisulfan, and the like. Radiation mutation-inducing agents include ultraviolet radiation, γ-irradiation, X-rays, and fast neutron bombardment. Genetic variation can also be introduced into a nucleic acid using, e.g., trimethylpsoralen with ultraviolet light. Random or targeted insertion of a mobile DNA element, e.g., a transposable element, is another suitable method for generating genetic variation. Genetic variations can be introduced into a nucleic acid during amplification in a cell-free in vitro system, e.g., using a polymerase chain reaction (PCR) technique such as error-prone PCR. Genetic variations can be introduced into a nucleic acid in vitro using DNA shuffling techniques (e.g., exon shuffling, domain swapping, and the like). Genetic variations can also be introduced into a nucleic acid as a result of a deficiency in a DNA repair enzyme in a cell, e.g., the presence in a cell of a mutant gene encoding a mutant DNA repair enzyme is expected to generate a high frequency of mutations (i.e., about 1 mutation/100 genes-1 mutation/10,000 genes) in the genome of the cell. Examples of genes encoding DNA repair enzymes include but are not limited to Mut H, Mut S, Mut L, and Mut U, and the homologs thereof in other species (e.g., MSH 1 6, PMS 1 2, MLH 1, GTBP, ERCC-1, and the like). Example descriptions of various methods for introducing genetic variations are provided in e.g., Stemple (2004) Nature 5:1-7; Chiang et al. (1993) PCR Methods Appl 2(3): 210-217; Stemmer (1994) Proc. Natl. Acad. Sci. USA 91:10747-10751; and U.S. Pat. Nos. 6,033,861, and 6,773,900.

Genetic variations introduced into microbes may be classified as transgenic, cisgenic, intragenomic, intrageneric, intergeneric, synthetic, evolved, rearranged, or SNPs.

Genetic variation may be introduced into numerous metabolic pathways within microbes to elicit improvements in the traits described above. Representative pathways include sulfur uptake pathways, glycogen biosynthesis, the glutamine regulation pathway, the molybdenum uptake pathway, the nitrogen fixation pathway, ammonia assimilation, ammonia excretion or secretion, Nitrogen uptake, glutamine biosynthesis, annamox, phosphate solubilization, organic acid transport, organic acid production, agglutinins production, reactive oxygen radical scavenging genes, Indole Acetic Acid biosynthesis, trehalose biosynthesis, plant cell wall degrading enzymes or pathways, root attachment genes, exopolysaccharide secretion, glutamate synthase pathway, iron uptake pathways, siderophore pathway, chitinase pathway, ACC deaminase, glutathione biosynthesis, phosphorous signaling genes, quorum quenching pathway, cytochrome pathways, hemoglobin pathway, bacterial hemoglobin-like pathway, small RNA rsmZ, rhizobitoxine biosynthesis, lapA adhesion protein, AHL quorum sensing pathway, phenazine biosynthesis, cyclic lipopeptide biosynthesis, and antibiotic production.

CRISPR/Cas9 (Clustered regularly interspaced short palindromic repeats)/CRISPR-associated (Cas) systems can be used to introduce desired mutations. CRISPR/Cas9 provide bacteria and archaea with adaptive immunity against viruses and plasmids by using CRISPR RNAs (crRNAs) to guide the silencing of invading nucleic acids. The Cas9 protein (or functional equivalent and/or variant thereof, i.e., Cas9-like protein) naturally contains DNA endonuclease activity that depends on the association of the protein with two naturally occurring or synthetic RNA molecules called crRNA and tracrRNA (also called guide RNAs). In some cases, the two molecules are covalently link to form a single molecule (also called a single guide RNA (“sgRNA”). Thus, the Cas9 or Cas9-like protein associates with a DNA-targeting RNA (which term encompasses both the two-molecule guide RNA configuration and the single-molecule guide RNA configuration), which activates the Cas9 or Cas9-like protein and guides the protein to a target nucleic acid sequence. If the Cas9 or Cas9-like protein retains its natural enzymatic function, it will cleave target DNA to create a double-stranded break, which can lead to genome alteration (i.e., editing: deletion, insertion (when a donor polynucleotide is present), replacement, etc.), thereby altering gene expression. Some variants of Cas9 (which variants are encompassed by the term Cas9-like) have been altered such that they have a decreased DNA cleaving activity (in some cases, they cleave a single strand instead of both strands of the target DNA, while in other cases, they have severely reduced to no DNA cleavage activity). Further exemplary descriptions of CRISPR systems for introducing genetic variation can be found in, e.g. U.S. Pat. No. 8,795,965.

As a cyclic amplification technique, polymerase chain reaction (PCR) mutagenesis uses mutagenic primers to introduce desired mutations. PCR is performed by cycles of denaturation, annealing, and extension. After amplification by PCR, selection of mutated DNA and removal of parental plasmid DNA can be accomplished by: 1) replacement of dCTP by hydroxymethylated-dCTP during PCR, followed by digestion with restriction enzymes to remove non-hydroxymethylated parent DNA only; 2) simultaneous mutagenesis of both an antibiotic resistance gene and the studied gene changing the plasmid to a different antibiotic resistance, the new antibiotic resistance facilitating the selection of the desired mutation thereafter; 3) after introducing a desired mutation, digestion of the parent methylated template DNA by restriction enzyme Dpnl which cleaves only methylated DNA, by which the mutagenized unmethylated chains are recovered; or 4) circularization of the mutated PCR products in an additional ligation reaction to increase the transformation efficiency of mutated DNA. Further description of exemplary methods can be found in e.g. U.S. Pat. Nos. 7,132,265, 6,713,285, 6,673,610, 6,391,548, 5,789,166, 5,780,270, 5,354,670, 5,071,743, and US20100267147.

Oligonucleotide-directed mutagenesis, also called site-directed mutagenesis, typically utilizes a synthetic DNA primer. This synthetic primer contains the desired mutation and is complementary to the template DNA around the mutation site so that it can hybridize with the DNA in the gene of interest. The mutation may be a single base change (a point mutation), multiple base changes, deletion, or insertion, or a combination of these. The single-strand primer is then extended using a DNA polymerase, which copies the rest of the gene. The gene thus copied contains the mutated site, and may then be introduced into a host cell as a vector and cloned. Finally, mutants can be selected by DNA sequencing to check that they contain the desired mutation.

Genetic variations can be introduced using error-prone PCR. In this technique the gene of interest is amplified using a DNA polymerase under conditions that are deficient in the fidelity of replication of sequence. The result is that the amplification products contain at least one error in the sequence. When a gene is amplified and the resulting product(s) of the reaction contain one or more alterations in sequence when compared to the template molecule, the resulting products are mutagenized as compared to the template. Another means of introducing random mutations is exposing cells to a chemical mutagen, such as nitrosoguanidine or ethyl methanesulfonate (Nestmann, Mutat Res 1975 June; 28(3):323-30), and the vector containing the gene is then isolated from the host.

Saturation mutagenesis is another form of random mutagenesis, in which one tries to generate all or nearly all possible mutations at a specific site, or narrow region of a gene. In a general sense, saturation mutagenesis is comprised of mutagenizing a complete set of mutagenic cassettes (wherein each cassette is, for example, 1-500 bases in length) in defined polynucleotide sequence to be mutagenized (wherein the sequence to be mutagenized is, for example, from 15 to 100,000 bases in length). Therefore, a group of mutations (e.g. ranging from 1 to 100 mutations) is introduced into each cassette to be mutagenized. A grouping of mutations to be introduced into one cassette can be different or the same from a second grouping of mutations to be introduced into a second cassette during the application of one round of saturation mutagenesis. Such groupings are exemplified by deletions, additions, groupings of particular codons, and groupings of particular nucleotide cassettes.

Fragment shuffling mutagenesis, also called DNA shuffling, is a way to rapidly propagate beneficial mutations. In an example of a shuffling process, DNAse is used to fragment a set of parent genes into pieces of e.g. about 50-100 bp in length. This is then followed by a polymerase chain reaction (PCR) without primers—DNA fragments with sufficient overlapping homologous sequence will anneal to each other and are then be extended by DNA polymerase. Several rounds of this PCR extension are allowed to occur, after some of the DNA molecules reach the size of the parental genes. These genes can then be amplified with another PCR, this time with the addition of primers that are designed to complement the ends of the strands. The primers may have additional sequences added to their 5′ ends, such as sequences for restriction enzyme recognition sites needed for ligation into a cloning vector. Further examples of shuffling techniques are provided in US20050266541.

Homologous recombination mutagenesis involves recombination between an exogenous DNA fragment and the targeted polynucleotide sequence. After a double-stranded break occurs, sections of DNA around the 5′ ends of the break are cut away in a process called resection. In the strand invasion step that follows, an overhanging 3′ end of the broken DNA molecule then “invades” a similar or identical DNA molecule that is not broken. The method can be used to delete a gene, remove exons, add a gene, and introduce point mutations. Homologous recombination mutagenesis can be permanent or conditional.

Typically, a recombination template is also provided. A recombination template may be a component of another vector, contained in a separate vector, or provided as a separate polynucleotide. In some embodiments, a recombination template is designed to serve as a template in homologous recombination, such as within or near a target sequence nicked or cleaved by a site-specific nuclease. A template polynucleotide may be of any suitable length, such as about or more than about 10, 15, 20, 25, 50, 75, 100, 150, 200, 500, 1000, or more nucleotides in length. In some embodiments, the template polynucleotide is complementary to a portion of a polynucleotide comprising the target sequence. When optimally aligned, a template polynucleotide might overlap with one or more nucleotides of a target sequences (e.g. about or more than about 1, 5, 10, 15, 20, 25, 30, 35, 40, 45, 50, 60, 70, 80, 90, 100 or more nucleotides). In some embodiments, when a template sequence and a polynucleotide comprising a target sequence are optimally aligned, the nearest nucleotide of the template polynucleotide is within about 1, 5, 10, 15, 20, 25, 50, 75, 100, 200, 300, 400, 500, 1000, 5000, 10000, or more nucleotides from the target sequence. Non-limiting examples of site-directed nucleases useful in methods of homologous recombination include zinc finger nucleases, CRISPR nucleases, TALE nucleases, and meganuclease. For a further description of the use of such nucleases, see e.g. U.S. Pat. No. 8,795,965 and US20140301990.

Mutagens that create primarily point mutations and short deletions, insertions, transversions, and/or transitions, including chemical mutagens or radiation, may be used to create genetic variations. Mutagens include, but are not limited to, ethyl methanesulfonate, methylmethane sulfonate, N-ethyl-N-nitrosurea, triethylmelamine, N-methyl-N-nitrosourea, procarbazine, chlorambucil, cyclophosphamide, diethyl sulfate, acrylamide monomer, melphalan, nitrogen mustard, vincristine, dimethylnitrosamine, N-methyl-N′-nitro-Nitrosoguanidine, nitrosoguanidine, 2-aminopurine, 7,12 dimethyl-benz(a)anthracene, ethylene oxide, hexamethylphosphoramide, bisulfan, diepoxyalkanes (diepoxyoctane, diepoxybutane, and the like), 2-methoxy-6-chloro-9[3-(ethyl-2-chloro-ethyl)aminopropylamino]acridine dihydrochloride and formaldehyde.

Introducing genetic variation may be an incomplete process, such that some bacteria in a treated population of bacteria carry a desired mutation while others do not. In some cases, it is desirable to apply a selection pressure so as to enrich for bacteria carrying a desired genetic variation. Traditionally, selection for successful genetic variants involved selection for or against some functionality imparted or abolished by the genetic variation, such as in the case of inserting antibiotic resistance gene or abolishing a metabolic activity capable of converting a non-lethal compound into a lethal metabolite. It is also possible to apply a selection pressure based on a polynucleotide sequence itself, such that only a desired genetic variation need be introduced (e.g. without also requiring a selectable marker). In this case, the selection pressure can comprise cleaving genomes lacking the genetic variation introduced to a target site, such that selection is effectively directed against the reference sequence into which the genetic variation is sought to be introduced. Typically, cleavage occurs within 100 nucleotides of the target site (e.g. within 75, 50, 25, 10, or fewer nucleotides from the target site, including cleavage at or within the target site). Cleaving may be directed by a site-specific nuclease selected from the group consisting of a Zinc Finger nuclease, a CRISPR nuclease, a TALE nuclease (TALEN), or a meganuclease. Such a process is similar to processes for enhancing homologous recombination at a target site, except that no template for homologous recombination is provided. As a result, bacteria lacking the desired genetic variation are more likely to undergo cleavage that, left unrepaired, results in cell death. Bacteria surviving selection may then be isolated for use in exposing to plants for assessing conferral of an improved trait.

A CRISPR nuclease may be used as the site-specific nuclease to direct cleavage to a target site. An improved selection of mutated microbes can be obtained by using Cas9 to kill non-mutated cells. Plants are then inoculated with the mutated microbes to re-confirm symbiosis and create evolutionary pressure to select for efficient symbionts. Microbes can then be re-isolated from plant tissues. CRISPR nuclease systems employed for selection against non-variants can employ similar elements to those described above with respect to introducing genetic variation, except that no template for homologous recombination is provided. Cleavage directed to the target site thus enhances death of affected cells.

Other options for specifically inducing cleavage at a target site are available, such as zinc finger nucleases, TALE nuclease (TALEN) systems, and meganuclease. Zinc-finger nucleases (ZFNs) are artificial DNA endonucleases generated by fusing a zinc finger DNA binding domain to a DNA cleavage domain. ZFNs can be engineered to target desired DNA sequences and this enables zinc-finger nucleases to cleave unique target sequences. When introduced into a cell, ZFNs can be used to edit target DNA in the cell (e.g., the cell's genome) by inducing double stranded breaks. Transcription activator-like effector nucleases (TALENs) are artificial DNA endonucleases generated by fusing a TAL (Transcription activator-like) effector DNA binding domain to a DNA cleavage domain. TALENS can be quickly engineered to bind practically any desired DNA sequence and when introduced into a cell, TALENs can be used to edit target DNA in the cell (e.g., the cell's genome) by inducing double strand breaks. Meganucleases (homing endonuclease) are endodeoxyribonucleases characterized by a large recognition site (double-stranded DNA sequences of 12 to 40 base pairs. Meganucleases can be used to replace, eliminate or modify sequences in a highly targeted way. By modifying their recognition sequence through protein engineering, the targeted sequence can be changed. Meganucleases can be used to modify all genome types, whether bacterial, plant or animal and are commonly grouped into four families: the LAGLIDADG family, the GIY-YIG family, the His-Cyst box family and the HNH family. Exemplary homing endonucleases include I-SceI, I-CeuI, PI-PspI, PI-Sce, I-SceIV, I-CsmI, I-PanI, I-SceII, I-PpoI, I-SceIII, I-CreI, I-TevI, I-TevII and I-TevIII.

Methods of the present disclosure may be employed to introduce or improve one or more of a variety of desirable traits. Examples of traits that may introduced or improved include: root biomass, root length, height, shoot length, leaf number, water use efficiency, overall biomass, yield, fruit size, grain size, photosynthesis rate, tolerance to drought, heat tolerance, salt tolerance, resistance to nematode stress, resistance to a fungal pathogen, resistance to a bacterial pathogen, resistance to a viral pathogen, level of a metabolite, and proteome expression. The desirable traits, including height, overall biomass, root and/or shoot biomass, seed germination, seedling survival, photosynthetic efficiency, transpiration rate, seed/fruit number or mass, plant grain or fruit yield, leaf chlorophyll content, photosynthetic rate, root length, or any combination thereof, can be used to measure growth, and compared with the growth rate of reference agricultural plants (e.g., plants without the improved traits) grown under identical conditions.

A preferred trait to be introduced or improved is nitrogen fixation, as described herein. In some cases, a plant resulting from the methods described herein exhibits a difference in the trait that is at least about 5% greater, for example at least about 5%, at least about 8%, at least about 10%, at least about 15%, at least about 20%, at least about 25%, at least about 30%, at least about 40%, at least about 50%, at least about 60%, at least about 75%, at least about 80%, at least about 80%, at least about 90%, or at least 100%, at least about 200%, at least about 300%, at least about 400% or greater than a reference agricultural plant grown under the same conditions in the soil. In additional examples, a plant resulting from the methods described herein exhibits a difference in the trait that is at least about 5% greater, for example at least about 5%, at least about 8%, at least about 10%, at least about 15%, at least about 20%, at least about 25%, at least about 30%, at least about 40%, at least about 50%, at least about 60%, at least about 75%, at least about 80%, at least about 80%, at least about 90%, or at least 100%, at least about 200%, at least about 300%, at least about 400% or greater than a reference agricultural plant grown under similar conditions in the soil.

The trait to be improved may be assessed under conditions including the application of one or more biotic or abiotic stressors. Examples of stressors include abiotic stresses (such as heat stress, salt stress, drought stress, cold stress, and low nutrient stress) and biotic stresses (such as nematode stress, insect herbivory stress, fungal pathogen stress, bacterial pathogen stress, and viral pathogen stress).

The trait improved by methods and compositions of the present disclosure may be nitrogen fixation, including in a plant not previously capable of nitrogen fixation. In some cases, bacteria isolated according to a method described herein produce 1% or more (e.g. 2%, 3%, 4%, 5%, 6%, 7%, 8%, 9%, 10%, 15%, 20%, or more) of a plant's nitrogen, which may represent an increase in nitrogen fixation capability of at least 2-fold (e.g. 3-fold, 4-fold, 5-fold, 6-fold, 7-fold, 8-fold, 9-fold, 10-fold, 20-fold, 50-fold, 100-fold, 1000-fold, or more) as compared to bacteria isolated from the first plant before introducing any genetic variation. In some cases, the bacteria produce 5% or more of a plant's nitrogen. The desired level of nitrogen fixation may be achieved after repeating the steps of introducing genetic variation, exposure to a plurality of plants, and isolating bacteria from plants with an improved trait one or more times (e.g. 1, 2, 3, 4, 5, 10, 15, 25, or more times). In some cases, enhanced levels of nitrogen fixation are achieved in the presence of fertilizer supplemented with glutamine, ammonia, or other chemical source of nitrogen. Methods for assessing degree of nitrogen fixation are known, examples of which are described herein.

Microbe breeding is a method to systematically identify and improve the role of species within the crop microbiome. The method comprises three steps: 1) selection of candidate species by mapping plant-microbe interactions and predicting regulatory networks linked to a particular phenotype, 2) pragmatic and predictable improvement of microbial phenotypes through intra-species crossing of regulatory networks and gene clusters, and 3) screening and selection of new microbial genotypes that produce desired crop phenotypes. To systematically assess the improvement of strains, a model is created that links colonization dynamics of the microbial community to genetic activity by key species. The model is used to predict genetic targets breeding and improve the frequency of selecting improvements in microbiome-encoded traits of agronomic relevance.

### Nitrogen Fixation

Described herein are methods of increasing nitrogen fixation in a plant, comprising exposing the plant to bacteria comprising one or more genetic variations introduced into one or more genes regulating nitrogen fixation, wherein the bacteria produce 1% or more of nitrogen in the plant (e.g. 2%, 5%, 10%, or more), which may represent a nitrogen-fixation capability of at least 2-fold as compared to the plant in the absence of the bacteria. The bacteria may produce the nitrogen in the presence of fertilizer supplemented with glutamine, urea, nitrates or ammonia. Genetic variations can be any genetic variation described herein, including examples provided above, in any number and any combination. The genetic variation may be introduced into a gene selected from the group consisting of nifA, nifL, ntrB, ntrC, glutamine synthetase, glnA, glnB, glnK, draT, amtB, glutaminase, glnD, glnE, nifJ, nifH, nifD, nifK, nifY, nifE, nifN, nifU, nifS, nifV, nifW, nifZ, nifM, nifF, nifB, and nifQ. The genetic variation may be a mutation that results in one or more of: increased expression or activity of nifA or glutaminase; decreased expression or activity of nifL, ntrB, glutamine synthetase, glnB, glnK, draT, amtB; decreased adenylyl-removing activity of GlnE; or decreased uridylyl-removing activity of GlnD. The genetic variation introduced into one or more bacteria of the methods disclosed herein may be a knock-out mutation or it may abolish a regulatory sequence of a target gene, or it may comprise insertion of a heterologous regulatory sequence, for example, insertion of a regulatory sequence found within the genome of the same bacterial species or genus. The regulatory sequence can be chosen based on the expression level of a gene in a bacterial culture or within plant tissue. The genetic variation may be produced by chemical mutagenesis. The plants grown in step (c) may be exposed to biotic or abiotic stressors.

The amount of nitrogen fixation that occurs in the plants described herein may be measured in several ways, for example by an acetylene-reduction (AR) assay. An acetylene-reduction assay can be performed in vitro or in vivo. Evidence that a particular bacterium is providing fixed nitrogen to a plant can include: 1) total plant N significantly increases upon inoculation, preferably with a concomitant increase in N concentration in the plant; 2) nitrogen deficiency symptoms are relieved under N-limiting conditions upon inoculation (which should include an increase in dry matter); 3) N₂ fixation is documented through the use of an ¹⁵N approach (which can be isotope dilution experiments, ¹⁵N₂ reduction assays, or ¹⁵N natural abundance assays); 4) fixed N is incorporated into a plant protein or metabolite; and 5) all of these effects are not be seen in non-inoculated plants or in plants inoculated with a mutant of the inoculum strain.

The wild-type nitrogen fixation regulatory cascade can be represented as a digital logic circuit where the inputs O2 and NH₄⁺ pass through a NOR gate, the output of which enters an AND gate in addition to ATP. In some embodiments, the methods disclosed herein disrupt the influence of NH₄⁺ on this circuit, at multiple points in the regulatory cascade, so that microbes can produce nitrogen even in fertilized fields. However, the methods disclosed herein also envision altering the impact of ATP or O2 on the circuitry, or replacing the circuitry with other regulatory cascades in the cell, or altering genetic circuits other than nitrogen fixation. Gene clusters can be re-engineered to generate functional products under the control of a heterologous regulatory system. By eliminating native regulatory elements outside of, and within, coding sequences of gene clusters, and replacing them with alternative regulatory systems, the functional products of complex genetic operons and other gene clusters can be controlled and/or moved to heterologous cells, including cells of different species other than the species from which the native genes were derived. Once re-engineered, the synthetic gene clusters can be controlled by genetic circuits or other inducible regulatory systems, thereby controlling the products' expression as desired. The expression cassettes can be designed to act as logic gates, pulse generators, oscillators, switches, or memory devices. The controlling expression cassette can be linked to a promoter such that the expression cassette functions as an environmental sensor, such as an oxygen, temperature, touch, osmotic stress, membrane stress, or redox sensor.

As an example, the nifL, nifA, nifT, and nifX genes can be eliminated from the nif gene cluster. Synthetic genes can be designed by codon randomizing the DNA encoding each amino acid sequence. Codon selection is performed, specifying that codon usage be as divergent as possible from the codon usage in the native gene. Proposed sequences are scanned for any undesired features, such as restriction enzyme recognition sites, transposon recognition sites, repetitive sequences, sigma 54 and sigma 70 promoters, cryptic ribosome binding sites, and rho independent terminators. Synthetic ribosome binding sites are chosen to match the strength of each corresponding native ribosome binding site, such as by constructing a fluorescent reporter plasmid in which the 150 bp surrounding a gene's start codon (from −60 to +90) is fused to a fluorescent gene. This chimera can be expressed under control of the Ptac promoter, and fluorescence measured via flow cytometry. To generate synthetic ribosome binding sites, a library of reporter plasmids using 150 bp (−60 to +90) of a synthetic expression cassette is generated. Briefly, a synthetic expression cassette can consist of a random DNA spacer, a degenerate sequence encoding an RBS library, and the coding sequence for each synthetic gene. Multiple clones are screened to identify the synthetic ribosome binding site that best matched the native ribosome binding site. Synthetic operons that consist of the same genes as the native operons are thus constructed and tested for functional complementation. A further exemplary description of synthetic operons is provided in US20140329326.

### Bacterial Species

Microbes useful in the methods and compositions disclosed herein may be obtained from any source. In some cases, microbes may be bacteria, archaea, protozoa or fungi. The microbes of this disclosure may be nitrogen fixing microbes, for example a nitrogen fixing bacteria, nitrogen fixing archaea, nitrogen fixing fungi, nitrogen fixing yeast, or nitrogen fixing protozoa. Microbes useful in the methods and compositions disclosed herein may be spore forming microbes, for example spore forming bacteria. In some cases, bacteria useful in the methods and compositions disclosed herein may be Gram positive bacteria or Gram negative bacteria. In some cases, the bacteria may be an endospore forming bacteria of the Firmicute phylum. In some cases, the bacteria may be a diazatroph. In some cases, the bacteria may not be a diazotroph.

The methods and compositions of this disclosure may be used with an archaea, such as, for example, Methanothermobacter thermoautotrophicus.

In some cases, bacteria which may be useful include, but are not limited to, Agrobacterium radiobacter, Bacillus acidocaldarius, Bacillus acidoterrestris, Bacillus agri, Bacillus aizawai, Bacillus albolactis, Bacillus alcalophidus, Bacillus alvei, Bacillus aininoglucosidicus, Bacillus aminovorans, Bacillus amylolyticus (also known as Paenibacillus amylolyticus) Bacillus amyloliquefaciens, Bacillus aneurinolyticus, Bacillus atrophaeus, Bacillus azotoformans, Bacillus badius, Bacillus cereus (synonyms. Bacillus endorhythmos, Bacillus medusa), Bacillus chitinosporus, Bacillus circulans. Bacillus coagulans, Bacillus endoparasiticus Bacillus fastidiosus, Bacillus firmus, Bacillus kurstali, Bacillus lacticola, Bacillus lactimorbus, Bacillus lactis, Bacillus laterosporus (also known as Brevibacillus laterosporus), Bacillus lautus, Bacillus lentimorbus, Bacillus lentus, Bacillus licheniformis, Bacillus maroccanus, Bacillus megateriun, Bacillus metiens, Bacillus mycoides, Bacillus natto, Bacillus nemnatocida, Bacillus nigrificans, Bacillus nigrum, Bacillus pantotenticts, Bacillus popillae, Bacillus psychrosaccharolyticus, Bacillus pumilus, Bacillus siamensis, Bacillus smithii, Bacillus sphaericus, Bacillus subtilis, Bacillus thuringiensis, Bacillus uniflagellatus, Bradyrhizobium japonicum, Brevibacillus brevis Brevibacillus laterosporus (formerly Bacillus laterosporus), Chromobacterium subtsugae. Delftia acidovorans, Lactobacillus acidophilus, Lysobacter antibioticus, Lysobacter enzymogenes, Paenibacillus alvei, Paenibacillus polymyxa, Paenibacillus popilliae (formerly Bacillus popilliae), Pantoea agglomerans, Pasteuria penetrans (formerly Bacillus penetrans), Pasteuria usgae, Pectobacterium carotovorm (formerly Erwinia carotovora), Pseudomonas aeruginosa, Pseudomonas aureofociens, Pseudomonas cepacia (formerly known as Burkholderia cepacia), Pseudomonas chlororaphis, Pseudomonas fluorescens, Pseudomonas proradix, Pseudomonas putida, Pseudomonas syringae, Serratia entomophila, Serratia marcescens, Streptomyces colombiensis, Streptomyces galbus, Streptomyces goshikiensis, Strepomyces griseoviridis, Streptomyces lavendulae, Streptomyces prasinus, Streptomyces saraceticus, Streptomyces venezuelae, Xanthomonas campestris, Xenorhabdus luminescens, Xenorhabdus nematophila, Rhodococcus globerulus AQ719 (NRRL Accession No. B-21663), Bacillus sp. AQ175 (ATCC Accession No. 55608), Bacillus sp. AQ 177 (ATCC Accession No, 55609), Bacillus sp. AQ178 (ATCC Accession No. 53522), and Streptomyces sp. strain NRRL Accession No. B-30145. In some cases the bacterium may be Azotobacter chroococcum, Methanosarcina barkeri, Klesiella pneumoniae, Azotobacter vinelandii, Rhodobacter spharoides, Rhodobacter capsulatus, Rhodobacter palustris, Rhodosporillum rubrum, Rhizobium leguminosarum or Rhizobium etli.

In some cases the bacterium may be a species of Clostridium, for example Clostridium pasteurianum, Clostridium beijerinckii, Clostridium perfringens, Clostridium tetani, Clostridium acetobutylicum.

In some cases, bacteria used with the methods and compositions of the present disclosure may be cyanobacteria. Examples of cyanobacterial genuses include Anabaena (for example Anagaena sp. PCC7120), Nostoc (for example Nostoc punctiforme), or Synechocystis (for example Synechocystis sp. PCC6803).

In some cases, bacteria used with the methods and compositions of the present disclosure may belong to the phylum Chlorobi, for example Chlorobium tepidum.

In some cases, microbes used with the methods and compositions of the present disclosure may comprise a gene homologous to a known NifH gene. Sequences of known NifH genes may be found in, for example, the Zehr lab NifH database, (wwwzehr.pmc.ucsc.edu/nifH_Database_Public/, Apr. 4, 2014), or the Buckley lab NifH database (www.css.cornell.edu/faculty/buckley/nifh.htm, and Gaby, John Christian. and Daniel H. Buckley. “A comprehensive aligned nifH gene database: a multipurpose tool for studies of nitrogen-fixing bacteria.” Database 2014 (2014): bau001.). In some cases, microbes used with the methods and compositions of the present disclosure may comprise a sequence which encodes a polypeptide with at least 60%, 70%, 80%, 85%, 90%, 95%, 96%, 96%, 98%, 99% or more than 99% sequence identity to a sequence from the Zehr lab NifH database, (wwwzehr.pmc.ucsc.edu/nifH_Database_Public/, Apr. 4, 2014). In some cases, microbes used with the methods and compositions of the present disclosure may comprise a sequence which encodes a polypeptide with at least 60%, 70%, 80%, 85%, 90%, 95%, 96%, 96%, 98%, 99% or more than 99% sequence identity to a sequence from the Buckley lab NifH database, (Gaby, John Christian, and Daniel H. Buckley. “A comprehensive aligned nifH gene database: a multipurpose tool for studies of nitrogen-fixing bacteria.” Database 2014 (2014): bau001.).

Microbes useful in the methods and compositions disclosed herein can be obtained by extracting microbes from surfaces or tissues of native plants; grinding seeds to isolate microbes; planting seeds in diverse soil samples and recovering microbes from tissues; or inoculating plants with exogenous microbes and determining which microbes appear in plant tissues. Non-limiting examples of plant tissues include a seed, seedling, leaf, cutting, plant, bulb or tuber. In some cases, bacteria are isolated from a seed. The parameters for processing samples may be varied to isolate different types of associative microbes, such as rhizospheric, epiphytes, or endophytes. Bacteria may also be sourced from a repository, such as environmental strain collections, instead of initially isolating from a first plant. The microbes can be genotyped and phenotyped, via sequencing the genomes of isolated microbes; profiling the composition of communities in planta; characterizing the transcriptomic functionality of communities or isolated microbes; or screening microbial features using selective or phenotypic media (e.g., nitrogen fixation or phosphate solubilization phenotypes). Selected candidate strains or populations can be obtained via sequence data; phenotype data; plant data (e.g., genome, phenotype, and/or yield data); soil data (e.g., pH, N/P/K content, and/or bulk soil biotic communities); or any combination of these.

The bacteria and methods of producing bacteria described herein may apply to bacteria able to self-propagate efficiently on the leaf surface, root surface, or inside plant tissues without inducing a damaging plant defense reaction, or bacteria that are resistant to plant defense responses. The bacteria described herein may be isolated by culturing a plant tissue extract or leaf surface wash in a medium with no added nitrogen. However, the bacteria may be unculturable, that is, not known to be culturable or difficult to culture using standard methods known in the art. The bacteria described herein may be an endophyte or an epiphyte or a bacterium inhabiting the plant rhizosphere (rhizospheric bacteria). The bacteria obtained after repeating the steps of introducing genetic variation, exposure to a plurality of plants, and isolating bacteria from plants with an improved trait one or more times (e.g. 1, 2, 3, 4, 5, 10, 15, 25, or more times) may be endophytic, epiphytic, or rhizospheric. Endophytes are organisms that enter the interior of plants without causing disease symptoms or eliciting the formation of symbiotic structures, and are of agronomic interest because they can enhance plant growth and improve the nutrition of plants (e.g., through nitrogen fixation). The bacteria can be a seed-borne endophyte. Seed-bome endophytes include bacteria associated with or derived from the seed of a grass or plant, such as a seed-bome bacterial endophyte found in mature, dry, undamaged (e.g., no cracks, visible fungal infection, or prematurely germinated) seeds. The seed-borne bacterial endophyte can be associated with or derived from the surface of the seed; alternatively, or in addition, it can be associated with or derived from the interior seed compartment (e.g., of a surface-sterilized seed). In some cases, a seed-borne bacterial endophyte is capable of replicating within the plant tissue, for example, the interior of the seed. Also, in some cases, the seed-bome bacterial endophyte is capable of surviving desiccation.

The bacterial isolated according to methods of the disclosure, or used in methods or compositions of the disclosure, can comprise a plurality of different bacterial taxa in combination. By way of example, the bacteria may include Proteobacteria (such as Pseudomonas, Enterobacter, Stenotrophomonas, Burkholderia, Rhizobium, Herbaspirillum, Pantoea, Serratia, Rahnella, Azospirillum, Azorhizobium, Azotobacter, Duganella, Delftia, Bradyrhizobiun, Sinorhizobium and Halomonas), Firmicutes (such as Bacillus, Paenibacillus, Lactobacillus, Mycoplasma, and Acetabacterium), and Actinobacteria (such as Streptomyces, Rhodacoccus, Microbacterium, and Curtobacterium). The bacteria used in methods and compositions of this disclosure may include nitrogen fixing bacterial consortia of two or more species. In some cases, one or more bacterial species of the bacterial consortia may be capable of fixing nitrogen. In some cases, one or more species of the bacterial consortia may facilitate or enhance the ability of other bacteria to fix nitrogen. The bacteria which fix nitrogen and the bacteria which enhance the ability of other bacteria to fix nitrogen may be the same or different. In some examples, a bacterial strain may be able to fix nitrogen when in combination with a different bacterial strain, or in a certain bacterial consortia, but may be unable to fix nitrogen in a monoculture. Examples of bacterial genuses which may be found in a nitrogen fixing bacterial consortia include, but are not limited to, Herbaspirillum, Azospirillum, Enterobacter, and Bacillus.

Bacteria that can be produced by the methods disclosed herein include Azotobacter sp., Bradyrhizobium sp., Klebsiella sp., and Sinorhizobium sp. In some cases, the bacteria may be selected from the group consisting of: Azotobacter vinelandii, Bradyrhizobium japonicum, Klebsiella pneumoniae, and Sinorhizobium meliloti. In some cases, the bacteria may be of the genus Enterobacter or Rahnella. In some cases, the bacteria may be of the genus Frankia, or Clostridium. Examples of bacteria of the genus Clostridium include, but are not limited to, Clostridium acetobutilicum, Clostridium pasteurianum, Clostridium beijerinckii, Clostridium perfringens, and Clostridium tetani. In some cases, the bacteria may be of the genus Paenibacillus, for example Paenibacillus azotofixans, Paenibacillus borealis, Paenibacillus durus, Paenibacillus macerans, Paenibacillus polymyxa, Paenibacillus alvei, Paenibacillus amylolyticus, Paenibacillus campinasensis, Paenibacillus chibensis, Paenibacillus glucanolyticus, Paenibacillus illinoisensis, Paenibacillus larvae subsp. Larvae, Paenibacillus larvae subsp. Pulvifaciens, Paenibacillus lautus, Paenibacillus macerans, Paenibacillus macquariensis, Paenibacillus macquariensis, Paenibacillus pabuli, Paenibacillus peoriae, or Paenibacillus polymyxa.

In some examples, bacteria isolated according to methods of the disclosure can be a member of one or more of the following taxa: Achromobacter, Acidithiobacillus, Acidovorax, Acidovoraz, Acinetobacter, Actinoplanes, Adlercreutzia, Aerococcus, Aeromonas, Afipia, Agromyces, Ancylobacter, Arthrobacter, Atopostipes, Azospirillum, Bacillus, Bdellovibrio, Beijerinckia, Bosea, Bradyrhizobium, Brevibacillus, Brevundimonas, Burkholderia, Candidatus Haloredivivus, Caulobacter, Cellulomonas, Cellvibrio, Chryseobacterium, Citrobacter, Clostridium, Coraliomargarita, Corynebacterium, Cupriavidus, Curtobacterium, Curvibacter, Deinococcus, Delftia, Desemzia, Devosia, Dokdonella, Dyella, Enhydrobacter, Enterobacter, Enterococcus, Erwinia, Escherichia, Escherichia/Shigella, Exiguobacterium, Ferroglobus, Filimonas, Finegoldia, Flavisolibacter, Flavobacterium, Frigoribacterium, Gluconacetobacter, Hafnia, Halobaculum, Halomonas, Halosimplex, Herbaspirillum, Hymenobacter, Klebsiella, Kocuria, Kosakonia, Lactobacillus, Leclercia, Lentzea, Luteibacter, Luteimonas, Massilia, Mesorhizobium, Methylobacterium, Microbacterium, Micrococcus, Microvirga, Mycobacterium, Neisseria, Nocardia, Oceanibaculum, Ochrobactrum, Okibacterium, Oligotropha, Oryzihumus, Oxalophagus, Paenibacillus, Panteoa, Pantoea, Pelomonas, Perlucidibaca, Plantibacter, Polynucleobacter, Propionibacterium, Propioniciclava, Pseudoclavibacter, Pseudomonas, Pseudonocardia, Pseudoxanthomonas, Psychrobacter, Ralstonia, Rheinheimera, Rhizobium, Rhodococcus, Rhodopseudomonas, Roseateles, Ruminococcus, Sebaldella, Sediminibacillus, Sediminibacterium, Serratia, Shigella, Shinella, Sinorhizobium, Sinosporangium, Sphingobacterium, Sphingomonas, Sphingopyxis, Sphingosinicella, Staphylococcus, 25 Stenotrophomonas, Strenotrophomonas, Streptococcus, Streptomyces, Stygiolobus, Sulfurisphaera, Tatumella, Tepidimonas, Thermomonas, Thiobacillus, Variovorax, WPS-2 genera incertae sedis, Xanthomonas, and Zimmermannella.

The bacteria may be obtained from any general terrestrial environment, including its soils, plants, fungi, animals (including invertebrates) and other biota, including the sediments, water and biota of lakes and rivers; from the marine environment, its biota and sediments (for example, sea water, marine muds, marine plants, marine invertebrates (for example, sponges), marine vertebrates (for example, fish)); the terrestrial and marine geosphere (regolith and rock, for example, crushed subterranean rocks, sand and clays); the cryosphere and its meltwater; the atmosphere (for example, filtered aerial dusts, cloud and rain droplets); urban, industrial and other man-made environments (for example, accumulated organic and mineral matter on concrete, roadside gutters, roof surfaces, and road surfaces).

The plants from which the bacteria are obtained may be a plant having one or more desirable traits, for example a plant which naturally grows in a particular environment or under certain conditions of interest. By way of example, a certain plant may naturally grow in sandy soil or sand of high salinity, or under extreme temperatures, or with little water, or it may be resistant to certain pests or disease present in the environment, and it may be desirable for a commercial crop to be grown in such conditions, particularly if they are, for example, the only conditions available in a particular geographic location. By way of further example, the bacteria may be collected from commercial crops grown in such environments, or more specifically from individual crop plants best displaying a trait of interest amongst a crop grown in any specific environment: for example the fastest-growing plants amongst a crop grown in saline-limiting soils, or the least damaged plants in crops exposed to severe insect damage or disease epidemic, or plants having desired quantities of certain metabolites and other compounds, including fiber content, oil content, and the like, or plants displaying desirable colors, taste or smell. The bacteria may be collected from a plant of interest or any material occurring in the environment of interest, including fungi and other animal and plant biota, soil, water, sediments, and other elements of the environment as referred to previously.

The bacteria may be isolated from plant tissue. This isolation can occur from any appropriate tissue in the plant, including for example root, stem and leaves, and plant reproductive tissues. By way of example, conventional methods for isolation from plants typically include the sterile excision of the plant material of interest (e.g. root or stem lengths, leaves), surface sterilization with an appropriate solution (e.g. 2% sodium hypochlorite), after which the plant material is placed on nutrient medium for microbial growth. Alternatively, the surface-sterilized plant material can be crushed in a sterile liquid (usually water) and the liquid suspension, including small pieces of the crushed plant material spread over the surface of a suitable solid agar medium, or media, which may or may not be selective (e.g. contain only phytic acid as a source of phosphorus). This approach is especially useful for bacteria which form isolated colonies and can be picked off individually to separate plates of nutrient medium, and further purified to a single species by well-known methods. Alternatively, the plant root or foliage samples may not be surface sterilized but only washed gently thus including surface-dwelling epiphytic microorganisms in the isolation process, or the epiphytic microbes can be isolated separately, by imprinting and lifting off pieces of plant roots, stem or leaves onto the surface of an agar medium and then isolating individual colonies as above. This approach is especially useful for bacteria, for example. Alternatively, the roots may be processed without washing off small quantities of soil attached to the roots, thus including microbes that colonize the plant rhizosphere. Otherwise, soil adhering to the roots can be removed, diluted and spread out onto agar of suitable selective and non-selective media to isolate individual colonies of rhizospheric bacteria.

Biologically pure cultures of Rahnella aquatilis and Enterobacter sacchari were deposited on Jul. 14, 2015 with the American Type Culture Collection (ATCC; an International Depositary Authority), Manassas, VA, USA, and assigned ATTC Patent Deposit Designation numbers PTA-122293 and PTA-122294, respectively. These deposits were made under the provisions of the Budapest Treaty on the International Recognition of the Deposit of Microorganisms for the Purpose of Patent Procedure and the Regulations (Budapest Treaty).

### Compositions

Compositions comprising bacteria or bacterial populations produced according to methods described herein and/or having characteristics as described herein can be in the form of a liquid, a foam, or a dry product. In some examples, a composition comprising bacterial populations may be in the form of a dry powder, a slurry of powder and water, or a flowable seed treatment.

The composition can be fabricated in bioreactors such as continuous stirred tank reactors, batch reactors, and on the farm. In some examples, compositions can be stored in a container, such as a jug or in mini bulk. In some examples, compositions may be stored within an object selected from the group consisting of a bottle, jar, ampule, package, vessel, bag, box, bin, envelope, carton, container, silo, shipping container, truck bed, and/or case.

Compositions may also be used to improve plant traits. In some examples, one or more compositions may be coated onto a seed. In some examples, one or more compositions may be coated onto a seedling. In some examples, one or more compositions may be coated onto a surface of a seed. In some examples, one or more compositions may be coated as a layer above a surface of a seed. In some examples, a composition that is coated onto a seed may be in liquid form, in dry product form, in foam form, in a form of a slurry of powder and water, or in a flowable seed treatment. In some examples, one or more compositions may be applied to a seed and/or seedling by spraying, immersing, coating, encapsulating, and/or dusting the seed and/or seedling with the one or more compositions. In some examples, multiple bacteria or bacterial populations can be coated onto a seed and/or a seedling of the plant. In some examples, at least two, at least three, at least four, at least five, at least six, at least seven, at least eight, at least nine, at least ten, or more than ten bacteria of a bacterial combination can be selected from one of the following genera: Acidovorax, Agrobacterium, Bacillus, Burkholderia, Chryseobacterium, Curtobacterium, Enterobacter, Escherichia, Methylobacterium, Paenibacillus, Pantoea, Pseudomonas, Ralstonia, Saccharibacillus, Sphingomonas, and Stenotrophomonas.

In some examples, at least two, at least three, at least four, at least five, at least six, at least seven, at least eight, at least nine, at least ten, or more than ten bacteria and bacterial populations of an endophytic combination are selected from one of the following families: Bacillaceae, Burkholderiaceae, Comamonadaceae, Enterobacteriaceae, Flavobacteriaceae, Methylobacteriaceae, Microbacteriaceae, Paenibacillileae, Pseudomonnaceae, Rhizobiaceae, Sphingomonadaceae, Xanthomonadaceae, Cladosporiaceae, Gnomoniaceae, Incertae sedis, Lasiosphaeriaceae, Netriaceae, and Pleosporaceae.

In some examples, at least two, at least three, at least four, at least five, at least six, at least seven, at least eight, at least night, at least ten, or more than ten bacteria and bacterial populations of an endophytic combination are selected from one of the following families: Bacillaceae, Burkholderiaceae, Comamonadaceae, Enterobacteriaceae, Flavobacteriaceae, Methylobacteriaceae, Microbacteriaceae, Paenibacillileae, Pseudomonnaceae, Rhizobiaceae, Sphingomonadaceae, Xanthomonadaceae, Cladosporiaceae, Gnomoniaceae, Incertae sedis, Lasiosphaeriaceae, Netriaceae, Pleosporaceae.

Examples of compositions may include seed coatings for commercially important agricultural crops, for example, sorghum, canola, tomato, strawberry, barley, rice, maize, and wheat. Examples of compositions can also include seed coatings for corn, soybean, canola, sorghum, potato, rice, vegetables, cereals, and oilseeds. Seeds as provided herein can be genetically modified organisms (GMO), non-GMO, organic, or conventional. In some examples, compositions may be sprayed on the plant aerial parts, or applied to the roots by inserting into furrows in which the plant seeds are planted, watering to the soil, or dipping the roots in a suspension of the composition. In some examples, compositions may be dehydrated in a suitable manner that maintains cell viability and the ability to artificially inoculate and colonize host plants. The bacterial species may be present in compositions at a concentration of between 10⁸ to 10¹⁰ CFU/ml. In some examples, compositions may be supplemented with trace metal ions, such as molybdenum ions, iron ions, manganese ions, or combinations of these ions. The concentration of ions in examples of compositions as described herein may between about 0.1 mM and about 50 mM. Some examples of compositions may also be formulated with a carrier, such as beta-glucan, carboxylmethyl cellulose (CMC), bacterial extracellular polymeric substance (EPS), sugar, animal milk, or other suitable carriers. In some examples, peat or planting materials can be used as a carrier, or biopolymers in which a composition is entrapped in the biopolymer can be used as a carrier.

The compositions comprising the bacterial populations described herein may be coated onto the surface of a seed. As such, compositions comprising a seed coated with one or more bacteria described herein are also contemplated. The seed coating can be formed by mixing the bacterial population with a porous, chemically inert granular carrier. Alternatively, the compositions may be inserted directly into the furrows into which the seed is planted or sprayed onto the plant leaves or applied by dipping the roots into a suspension of the composition. An effective amount of the composition can be used to populate the sub-soil region adjacent to the roots of the plant with viable bacterial growth, or populate the leaves of the plant with viable bacterial growth. In general, an effective amount is an amount sufficient to result in plants with improved traits (e.g. a desired level of nitrogen fixation).

Bacterial compositions described herein can be formulated using an agriculturally acceptable carrier. The formulation useful for these embodiments may include at least one member selected from the group consisting of a tackifier, a microbial stabilizer, a fungicide, an antibacterial agent, a preservative, a stabilizer, a surfactant, an anti-complex agent, an herbicide, a nematicide, an insecticide, a plant growth regulator, a fertilizer, a rodenticide, a dessicant, a bactericide, a nutrient, or any combination thereof. In some examples, compositions may be shelf-stable. For example, any of the compositions described herein can include an agriculturally acceptable carrier (e.g., one or more of a fertilizer such as a non-naturally occurring fertilizer, an adhesion agent such as a non-naturally occurring adhesion agent, and a pesticide such as a non-naturally occurring pesticide). A non-naturally occurring adhesion agent can be, for example, a polymer, copolymer, or synthetic wax. For example, any of the coated seeds, seedlings, or plants described herein can contain such an agriculturally acceptable carrier in the seed coating. In any of the compositions or methods described herein, an agriculturally acceptable carrier can be or can include a non-naturally occurring compound (e.g., a non-naturally occurring fertilizer, a non-naturally occurring adhesion agent such as a polymer, copolymer, or synthetic wax, or a non-naturally occurring pesticide). Non-limiting examples of agriculturally acceptable carriers are described below. Additional examples of agriculturally acceptable carriers are known in the art.

In some cases, bacteria are mixed with an agriculturally acceptable carrier. The carrier can be a solid carrier or liquid carrier, and in various forms including microspheres, powders, emulsions and the like. The carrier may be any one or more of a number of carriers that confer a variety of properties, such as increased stability, wettability, or dispersability. Wetting agents such as natural or synthetic surfactants, which can be nonionic or ionic surfactants, or a combination thereof can be included in the composition. Water-in-oil emulsions can also be used to formulate a composition that includes the isolated bacteria (see, for example, U.S. Pat. No. 7,485,451). Suitable formulations that may be prepared include wettable powders, granules, gels, agar strips or pellets, thickeners, and the like, microencapsulated particles, and the like, liquids such as aqueous flowables, aqueous suspensions, water-in-oil emulsions, etc. The formulation may include grain or legume products, for example, ground grain or beans, broth or flour derived from grain or beans, starch, sugar, or oil.

In some embodiments, the agricultural carrier may be soil or a plant growth medium. Other agricultural carriers that may be used include water, fertilizers, plant-based oils, humectants, or combinations thereof. Alternatively, the agricultural carrier may be a solid, such as diatomaceous earth, loam, silica, alginate, clay, bentonite, vermiculite, seed cases, other plant and animal products, or combinations, including granules, pellets, or suspensions. Mixtures of any of the aforementioned ingredients are also contemplated as carriers, such as but not limited to, pesta (flour and kaolin clay), agar or flour-based pellets in loam, sand, or clay, etc. Formulations may include food sources for the bacteria, such as barley, rice, or other biological materials such as seed, plant parts, sugar cane bagasse, hulls or stalks from grain processing, ground plant material or wood from building site refuse, sawdust or small fibers from recycling of paper, fabric, or wood.

For example, a fertilizer can be used to help promote the growth or provide nutrients to a seed, seedling, or plant. Non-limiting examples of fertilizers include nitrogen, phosphorous, potassium, calcium, sulfur, magnesium, boron, chloride, manganese, iron, zinc, copper, molybdenum, and selenium (or a salt thereof). Additional examples of fertilizers include one or more amino acids, salts, carbohydrates, vitamins, glucose, NaCl, yeast extract, NH₄H₂PO₄, (NH₄)₂SO₄, glycerol, valine, L-leucine, lactic acid, propionic acid, succinic acid, malic acid, citric acid, KH tartrate, xylose, lyxose, and lecithin. In one embodiment, the formulation can include a tackifier or adherent (referred to as an adhesive agent) to help bind other active agents to a substance (e.g., a surface of a seed). Such agents are useful for combining bacteria with carriers that can contain other compounds (e.g., control agents that are not biologic), to yield a coating composition. Such compositions help create coatings around the plant or seed to maintain contact between the microbe and other agents with the plant or plant part. In one embodiment, adhesives are selected from the group consisting of: alginate, gums, starches, lecithins, formononetin, polyvinyl alcohol, alkali formononetinate, hesperetin, polyvinyl acetate, cephalins, Gum Arabic, Xanthan Gum, Mineral Oil, Polyethylene Glycol (PEG), Polyvinyl pyrrolidone (PVP), Arabino-galactan, Methyl Cellulose, PEG 400, Chitosan, Polyacrylamide, Polyacrylate, Polyacrylonitrile, Glycerol, Triethylene glycol, Vinyl Acetate, Gellan Gum, Polystyrene, Polyvinyl, Carboxymethyl cellulose, Gum Ghatti, and polyoxyethylene-polyoxybutylene block copolymers.

In some embodiments, the adhesives can be, e.g. a wax such as carnauba wax, beeswax, Chinese wax, shellac wax, spermaceti wax, candelilla wax, castor wax, ouricury wax, and rice bran wax, a polysaccharide (e.g., starch, dextrins, maltodextrins, alginate, and chitosans), a fat, oil, a protein (e.g., gelatin and zeins), gum arables, and shellacs. Adhesive agents can be non-naturally occurring compounds, e.g., polymers, copolymers, and waxes. For example, non-limiting examples of polymers that can be used as an adhesive agent include: polyvinyl acetates, polyvinyl acetate copolymers, ethylene vinyl acetate (EVA) copolymers, polyvinyl alcohols, polyvinyl alcohol copolymers, celluloses (e.g., ethylcelluloses, methylcelluloses, hydroxymethylcelluloses, hydroxypropylcelluloses, and carboxymethylcelluloses), polyvinylpyrolidones, vinyl chloride, vinylidene chloride copolymers, calcium lignosulfonates, acrylic copolymers, polyvinylacrylates, polyethylene oxide, acylamide polymers and copolymers, polyhydroxyethyl acrylate, methylacrylamide monomers, and polychloroprene.

In some examples, one or more of the adhesion agents, anti-fungal agents, growth regulation agents, and pesticides (e.g., insecticide) are non-naturally occurring compounds (e.g., in any combination). Additional examples of agriculturally acceptable carriers include dispersants (e.g., polyvinylpyrrolidone/vinyl acetate PVPIVA 5-630), surfactants, binders, and filler agents.

The formulation can also contain a surfactant. Non-limiting examples of surfactants include nitrogen-surfactant blends such as Prefer 28 (Cenex), Surf-N (US), Inhance (Brandt), P-28 (Wilfarm) and Patrol (Helena); esterified seed oils include Sun-It II (AmCy), MSO (UAP), Scoil (Agsco), Hasten (Wilfarm) and Mes-100 (Drexel); and organo-silicone surfactants include Silwet L77 (UAP), Silikin (Terra), Dyne-Amic (Helena), Kinetic (Helena), Sylgard 309 (Wilbur-Ellis) and Century (Precision). In one embodiment, the surfactant is present at a concentration of between 0.01% v/v to 10% v/v. In another embodiment, the surfactant is present at a concentration of between 0.1% v/v to 1% v/v.

In certain cases, the formulation includes a microbial stabilizer. Such an agent can include a desiccant, which can include any compound or mixture of compounds that can be classified as a desiccant regardless of whether the compound or compounds are used in such concentrations that they in fact have a desiccating effect on a liquid inoculant. Such desiccants are ideally compatible with the bacterial population used, and should promote the ability of the microbial population to survive application on the seeds and to survive desiccation. Examples of suitable desiccants include one or more of trehalose, sucrose, glycerol, and Methylene glycol. Other suitable desiccants include, but are not limited to, non reducing sugars and sugar alcohols (e.g., mannitol or sorbitol). The amount of desiccant introduced into the formulation can range from about 5% to about 50% by weight/volume, for example, between about 10% to about 40%, between about 15% to about 35%, or between about 20% to about 30%. In some cases, it is advantageous for the formulation to contain agents such as a fungicide, an antibacterial agent, an herbicide, a nematicide, an insecticide, a plant growth regulator, a rodenticide, bactericide, or a nutrient. In some examples, agents may include protectants that provide protection against seed surface-bome pathogens. In some examples, protectants may provide some level of control of soil-borne pathogens. In some examples, protectants may be effective predominantly on a seed surface.

In some examples, a fungicide may include a compound or agent, whether chemical or biological, that can inhibit the growth of a fungus or kill a fungus. In some examples, a fungicide may include compounds that may be fungistatic or fungicidal. In some examples, fungicide can be a protectant, or agents that are effective predominantly on the seed surface, providing protection against seed surface-borne pathogens and providing some level of control of soil-bome pathogens. Non-limiting examples of protectant fungicides include captan, maneb, thiram, or fludioxonil.

In some examples, fungicide can be a systemic fungicide, which can be absorbed into the emerging seedling and inhibit or kill the fungus inside host plant tissues. Systemic fungicides used for seed treatment include, but are not limited to the following: azoxystrobin, carboxin, mefenoxam, metalaxyl, thiabendazole, trifloxystrobin, and various triazole fungicides, including difenoconazole, ipconazole, tebuconazole, and triticonazole. Mefenoxam and metalaxyl are primarily used to target the water mold fungi Pythium and Phytophthora. Some fungicides are preferred over others, depending on the plant species, either because of subtle differences in sensitivity of the pathogenic fungal species, or because of the differences in the fungicide distribution or sensitivity of the plants. In some examples, fungicide can be a biological control agent, such as a bacterium or fungus. Such organisms may be parasitic to the pathogenic fungi, or secrete toxins or other substances which can kill or otherwise prevent the growth of fungi. Any type of fungicide, particularly ones that are commonly used on plants, can be used as a control agent in a seed composition.

In some examples, the seed coating composition comprises a control agent which has antibacterial properties. In one embodiment, the control agent with antibacterial properties is selected from the compounds described herein elsewhere. In another embodiment, the compound is Streptomycin, oxytetracycline, oxolinic acid, or gentamicin. Other examples of antibacterial compounds which can be used as part of a seed coating composition include those based on dichlorophene and benzylalcohol hemi formal (Proxel® from ICI or Acticide® RS from Thor Chemie and Kathon® MK 25 from Rohm & Haas) and isothiazolinone derivatives such as alkylisothiazolinones and benzisothiazolinones (Acticide® MBS from Thor Chemie).

In some examples, growth regulator is selected from the group consisting of. Abscisic acid, amidochlor, ancymidol, 6-benzylaminopurine, brassinolide, butralin, chlormequat (chlormequat chloride), choline chloride, cyclanilide, daminozide, dikegulac, dimethipin, 2,6-dimethylpuridine, ethephon, flumetralin, flurprimidol, fluthiacet, forchlorfenuron, gibberellic acid, inabenfide, indole-3-acetic acid, maleic hydrazide, mefluidide, mepiquat (mepiquat chloride), naphthaleneacetic acid, N-6-benzyladenine, paclobutrazol, prohexadione phosphorotrithioate, 2,3,5-tri-iodobenzoic acid, trinexapac-ethyl and uniconazole. Additional non-limiting examples of growth regulators include brassinosteroids, cytokinines (e.g., kinetin and zeatin), auxins (e.g., indolylacetic acid and indolylacetyl aspartate), flavonoids and isoflavanoids (e.g., formononetin and diosmetin), phytoaixins (e.g., glyceolline), and phytoalexin-inducing oligosaccharides (e.g., pectin, chitin, chitosan, polygalacuronic acid, and oligogalacturonic acid), and gibellerins. Such agents are ideally compatible with the agricultural seed or seedling onto which the formulation is applied (e.g., it should not be deleterious to the growth or health of the plant). Furthermore, the agent is ideally one which does not cause safety concerns for human, animal or industrial use (e.g., no safety issues, or the compound is sufficiently labile that the commodity plant product derived from the plant contains negligible amounts of the compound).

Some examples of nematode-antagonistic biocontrol agents include ARF18; 30 Arthrobotrys spp.; Chaetomium spp.; Cylindrocarpon spp.; Exophilia spp.; Fusarium spp.; Gliocladium spp.; Hirsutella spp.; Lecanicillium spp.; Monacrosporium spp.; Myrothecium spp.; Neocosmospora spp.; Paecilomyces spp.; Pochonia spp.; Stagonospora spp.; vesicular-arbuscular mycorrhizal fungi, Burkholderia spp.; Pasteuria spp., Brevibacillus spp.; Pseudomonas spp.; and Rhizobacteria. Particularly preferred nematode-antagonistic biocontrol agents include ARF18, Arthrobotrys oligospora, Arthrobotrys dactyloides, Chaetomium globosum, Cylindrocarpon heteronema, Exophilia jeanselmei, Exophilia pisciphila, Fusarium aspergilus, Fusarium solani, Gliocladium catenulatum, Gliocladium roseum, Gliocladium vixens, Hirsutella rhossiliensis, Hirsutella minnesotensis, Lecanicillium lecanii, Monacrosporium drechsleri, Monacrosporium gephyropagum, Myrotehcium verrucaria, Neocosmospora vasinfecta, Paecilomyces lilacinus, Pochonia chlamydosporia, Stagonospora heteroderae, Stagonospora phaseoli, vesicular-arbuscular mycorrhizal fungi, Burkholderia cepacia, Pasteuria penetrans, Pasteuria thornei, Pasteuria nishizawae, Pasteuria ramosa, Pastrueia usage, Brevibacillus laterosporus strain G4, Pseudomonas fluorescens and Rhizobacteria.

Some examples of nutrients can be selected from the group consisting of a nitrogen fertilizer including, but not limited to Urea, Ammonium nitrate, Ammonium sulfate, Non-pressure nitrogen solutions, Aqua ammonia, Anhydrous ammonia, Ammonium thiosulfate, Sulfur-coated urea, Urea-formaldehydes, IBDU, Polymer-coated urea, Calcium nitrate, Ureaform, and Methylene urea, phosphorous fertilizers such as Diammonium phosphate, Monoammonium phosphate, Ammonium polyphosphate, Concentrated superphosphate and Triple superphosphate, and potassium fertilizers such as Potassium chloride, Potassium sulfate, Potassium-magnesium sulfate, Potassium nitrate. Such compositions can exist as free salts or ions within the seed coat composition. Alternatively, nutrients/fertilizers can be complexed or chelated to provide sustained release over time.

Some examples of rodenticides may include selected from the group of substances consisting of 2-isovalerylindan-1,3-dione, 4-(quinoxalin-2-ylamino) benzenesulfonamide, alpha-chlorohydrin, aluminum phosphide, antu, arsenous oxide, barium carbonate, bisthiosemi, brodifacoum, bromadiolone, bromethalin, calcium cyanide, chloralose, chlorophacinone, cholecalciferol, coumachlor, coumafuryl, coumatetralyl, crimidine, difenacoum, difethialone, diphacinone, ergocalciferol, flocoumafen, fluoroacetamide, flupropadine, flupropadine hydrochloride, hydrogen cyanide, iodomethane, lindane, magnesium phosphide, methyl bromide, norbormide, phosacetim, phosphine, phosphorus, pindone, potassium arsenite, pyrinuron, scilliroside, sodium arsenite, sodium cyanide, sodium fluoroacetate, strychnine, thallium sulfate, warfarin and zinc phosphide.

In the liquid form, for example, solutions or suspensions, bacterial populations can be mixed or suspended in water or in aqueous solutions. Suitable liquid diluents or carriers include water, aqueous solutions, petroleum distillates, or other liquid carriers.

Solid compositions can be prepared by dispersing the bacterial populations in and on an appropriately divided solid carrier, such as peat, wheat, bran, vermiculite, clay, talc, bentonite, diatomaceous earth, fuller's earth, pasteurized soil, and the like. When such formulations are used as wettable powders, biologically compatible dispersing agents such as non-ionic, anionic, amphoteric, or cationic dispersing and emulsifying agents can be used.

The solid carriers used upon formulation include, for example, mineral carriers such as kaolin clay, pyrophyllite, bentonite, montmorillonite, diatomaceous earth, acid white soil, vermiculite, and pearlite, and inorganic salts such as ammonium sulfate, ammonium phosphate, ammonium nitrate, urea, ammonium chloride, and calcium carbonate. Also, organic fine powders such as wheat flour, wheat bran, and rice bran may be used. The liquid carriers include vegetable oils such as soybean oil and cottonseed oil, glycerol, ethylene glycol, polyethylene glycol, propylene glycol, polypropylene glycol, etc.

### Application of Bacterial Populations on Crops

The composition of the bacteria or bacterial population described herein can be applied in furrow, in talc, or as seed treatment. The composition can be applied to a seed package in bulk, mini bulk, in a bag, or in talc.

The planter can plant the treated seed and grows the crop according to conventional ways, twin row, or ways that do not require tilling. The seeds can be distributed using a control hopper or an individual hopper. Seeds can also be distributed using pressurized air or manually. Seed placement can be performed using variable rate technologies. Additionally, application of the bacteria or bacterial population described herein may be applied using variable rate technologies. In some examples, the bacteria can be applied to seeds of corn, soybean, canola, Sorghum, potato, rice, vegetables, cereals, pseudocereals, and oilseeds. Examples of cereals may include barley, fonio, oats, palmer's grass, rye, pearl millet, Sorghum, spelt, teff, triticale, and wheat. Examples of pseudocereals may include breadnut, buckwheat, cattail, chia, flax, grain amaranth, hanza, quinoa, and sesame. In some examples, seeds can be genetically modified organisms (GMO), non-GMO, organic or conventional.

Additives such as micro-fertilizer, PGR, herbicide, insecticide, and fungicide can be used additionally to treat the crops. Examples of additives include crop protectants such as insecticides, nematicides, fungicide, enhancement agents such as colorants, polymers, pelleting, priming, and disinfectants, and other agents such as inoculant, PGR, softener, and micronutrients. PGRs can be natural or synthetic plant hormones that affect root growth, flowering, or stem elongation. PGRs can include auxins, gibberellins, cytokinins, ethylene, and abscisic acid (ABA).

The composition can be applied in furrow in combination with liquid fertilizer. In some examples, the liquid fertilizer may be held in tanks. NPK fertilizers contain macronutrients of sodium, phosphorous, and potassium.

The composition may improve plant traits, such as promoting plant growth, maintaining high chlorophyll content in leaves, increasing fruit or seed numbers, and increasing fruit or seed unit weight. Methods of the present disclosure may be employed to introduce or improve one or more of a variety of desirable traits. Examples of traits that may introduced or improved include: root biomass, root length, height, shoot length, leaf number, water use efficiency, overall biomass, yield, fruit size, grain size, photosynthesis rate, tolerance to drought, heat tolerance, salt tolerance, tolerance to low nitrogen stress, nitrogen use efficiency, resistance to nematode stress, resistance to a fungal pathogen, resistance to a bacterial pathogen, resistance to a viral pathogen, level of a metabolite, modulation in level of a metabolite, proteome expression. The desirable traits, including height, overall biomass, root and/or shoot biomass, seed germination, seedling survival, photosynthetic efficiency, transpiration rate, seed/fruit number or mass, plant grain or fruit yield, leaf chlorophyll content, photosynthetic rate, root length, or any combination thereof, can be used to measure growth, and compared with the growth rate of reference agricultural plants (e.g., plants without the introduced and/or improved traits) grown under identical conditions. In some examples, the desirable traits, including height, overall biomass, root and/or shoot biomass, seed germination, seedling survival, photosynthetic efficiency, transpiration rate, seed/fruit number or mass, plant grain or fruit yield, leaf chlorophyll content, photosynthetic rate, root length, or any combination thereof, can be used to measure growth, and compared with the growth rate of reference agricultural plants (e.g., plants without the introduced and/or improved traits) grown under similar conditions.

An agronomic trait to a host plant may include, but is not limited to, the following: altered oil content, altered protein content, altered seed carbohydrate composition, altered seed oil composition, and altered seed protein composition, chemical tolerance, cold tolerance, delayed senescence, disease resistance, drought tolerance, ear weight, growth improvement, health enhancement, heat tolerance, herbicide tolerance, herbivore resistance improved nitrogen fixation, improved nitrogen utilization, improved root architecture, improved water use efficiency, increased biomass, increased root length, increased seed weight, increased shoot length, increased yield, increased yield under water-limited conditions, kernel mass, kernel moisture content, metal tolerance, number of ears, number of kernels per ear, number of pods, nutrition enhancement, pathogen resistance, pest resistance, photosynthetic capability improvement, salinity tolerance, stay-green, vigor improvement, increased dry weight of mature seeds, increased fresh weight of mature seeds, increased number of mature seeds per plant, increased chlorophyll content, increased number of pods per plant, increased length of pods per plant, reduced number of wilted leaves per plant, reduced number of severely wilted leaves per plant, and increased number of non-wilted leaves per plant, a detectable modulation in the level of a metabolite, a detectable modulation in the level of a transcript, and a detectable modulation in the proteome, compared to an isoline plant grown from a seed without said seed treatment formulation

In some cases, plants are inoculated with bacteria or bacterial populations that are isolated from the same species of plant as the plant element of the inoculated plant. For example, an bacteria or bacterial population that is normally found in one variety of Zea mays (corn) is associated with a plant element of a plant of another variety of Zea mays that in its natural state lacks said bacteria and bacterial populations. In one embodiment, the bacteria and bacterial populations is derived from a plant of a related species of plant as the plant element of the inoculated plant. For example, an bacteria and bacterial populations that is normally found in Zea diploperennisIltis et al., (diploperennial teosinte) is applied to a Zea mays (corn), or vice versa. In some cases, plants are inoculated with bacteria and bacterial populations that are heterologous to the plant element of the inoculated plant. In one embodiment, the bacteria and bacterial populations is derived from a plant of another species. For example, an bacteria and bacterial populations that is normally found in dicots is applied to a monocot plant (e.g., inoculating corn with a soybean-derived bacteria and bacterial populations), or vice versa. In other cases, the bacteria and bacterial populations to be inoculated onto a plant is derived from a related species of the plant that is being inoculated. In one embodiment, the bacteria and bacterial populations is derived from a related taxon, for example, from a related species. The plant of another species can be an agricultural plant. In another embodiment, the bacteria and bacterial populations is part of a designed composition inoculated into any host plant element.

In some examples, the bacteria or bacterial population is exogenous wherein the bacteria and bacterial population is isolated from a different plant than the inoculated plant. For example, in one embodiment, the bacteria or bacterial population can be isolated from a different plant of the same species as the inoculated plant. In some cases, the bacteria or bacterial population can be isolated from a species related to the inoculated plant.

In some examples, the bacteria and bacterial populations described herein are capable of moving from one tissue type to another. For example, the present invention's detection and isolation of bacteria and bacterial populations within the mature tissues of plants after coating on the exterior of a seed demonstrates their ability to move from seed exterior into the vegetative tissues of a maturing plant. Therefore, in one embodiment, the population of bacteria and bacterial populations is capable of moving from the seed exterior into the vegetative tissues of a plant. In one embodiment, the bacteria and bacterial populations that is coated onto the seed of a plant is capable, upon germination of the seed into a vegetative state, of localizing to a different tissue of the plant. For example, bacteria and bacterial populations can be capable of localizing to any one of the tissues in the plant, including: the root, adventitious root, seminal 5 root, root hair, shoot, leaf, flower, bud, tassel, meristem, pollen, pistil, ovaries, stamen, fruit, stolon, rhizome, nodule, tuber, trichome, guard cells, hydathode, petal, sepal, glume, rachis, vascular cambium, phloem, and xylem. In one embodiment, the bacteria and bacterial populations is capable of localizing to the root and/or the root hair of the plant. In another embodiment, the bacteria and bacterial populations is capable of localizing to the photosynthetic tissues, for example, leaves and shoots of the plant. In other cases, the bacteria and bacterial populations is localized to the vascular tissues of the plant, for example, in the xylem and phloem. In still another embodiment, the bacteria and bacterial populations is capable of localizing to the reproductive tissues (flower, pollen, pistil, ovaries, stamen, fruit) of the plant. In another embodiment, the bacteria and bacterial populations is capable of localizing to the root, shoots, leaves and reproductive tissues of the plant. In still another embodiment, the bacteria and bacterial populations colonizes a fruit or seed tissue of the plant. In still another embodiment, the bacteria and bacterial populations is able to colonize the plant such that it is present in the surface of the plant (i.e., its presence is detectably present on the plant exterior, or the episphere of the plant). In still other embodiments, the bacteria and bacterial populations is capable of localizing to substantially all, or all, tissues of the plant. In certain embodiments, the bacteria and bacterial populations is not localized to the root of a plant. In other cases, the bacteria and bacterial populations is not localized to the photosynthetic tissues of the plant.

The effectiveness of the compositions can also be assessed by measuring the relative maturity of the crop or the crop heating unit (CHU). For example, the bacterial population can be applied to corn, and corn growth can be assessed according to the relative maturity of the corn kernel or the time at which the corn kernel is at maximum weight. The crop heating unit (CHU) can also be used to predict the maturation of the corn crop. The CHU determines the amount of heat accumulation by measuring the daily maximum temperatures on crop growth.

In examples, bacterial may localize to any one of the tissues in the plant, including: the root, adventitious root, seminal root, root hair, shoot, leaf, flower, bud tassel, meristem, pollen, pistil, ovaries, stamen, fruit, stolon, rhizome, nodule, tuber, trichome, guard cells, hydathode, petal, sepal, glume, rachis, vascular cambium, phloem, and xylem. In another embodiment, the bacteria or bacterial population is capable of localizing to the photosynthetic tissues, for example, leaves and shoots of the plant. In other cases, the bacteria and bacterial populations is localized to the vascular tissues of the plant, for example, in the xylem and phloem. In another embodiment, the bacteria or bacterial population is capable of localizing to reproductive tissues (flower, pollen, pistil, ovaries, stamen, or fruit) of the plant. In another embodiment, the bacteria and bacterial populations is capable of localizing to the root, shoots, leaves and reproductive tissues of the plant. In another embodiment, the bacteria or bacterial population colonizes a fruit or seed tissue of the plant. In still another embodiment, the bacteria or bacterial population is able to colonize the plant such that it is present in the surface of the plant. In another embodiment, the bacteria or bacterial population is capable of localizing to substantially all, or all, tissues of the plant. In certain embodiments, the bacteria or bacterial population is not localized to the root of a plant. In other cases, the bacteria and bacterial populations is not localized to the photosynthetic tissues of the plant.

The effectiveness of the bacterial compositions applied to crops can be assessed by measuring various features of crop growth including, but not limited to, planting rate, seeding vigor, root strength, drought tolerance, plant height, dry down, and test weight.

### Plant Species

The methods and bacteria described herein are suitable for any of a variety of plants, such as plants in the genera Hordeum, Oryza, Zea, and Triticeae. Other non-limiting examples of suitable plants include mosses, lichens, and algae. In some cases, the plants have economic, social and/or environmental value, such as food crops, fiber crops, oil crops, plants in the forestry or pulp and paper industries, feedstock for biofuel production and/or ornamental plants. In some examples, plants may be used to produce economically valuable products such as a grain, a flour, a starch, a syrup, a meal, an oil, a film, a packaging, a nutraceutical product, a pulp, an animal feed, a fish fodder, a bulk material for industrial chemicals, a cereal product, a processed human-food product, a sugar, an alcohol, and/or a protein. Non-limiting examples of crop plants include maize, rice, wheat, barley, sorghum, millet, oats, rye triticale, buckwheat, sweet corn, sugar cane, onions, tomatoes, strawberries, and asparagus.

In some examples, plants that may be obtained or improved using the methods and composition disclosed herein may include plants that are important or interesting for agriculture, horticulture, biomass for the production of biofuel molecules and other chemicals, and/or forestry. Some examples of these plants may include pineapple, banana, coconut, lily, grasspeas, alfalfa, tomatillo, melon, chickpea, chicory, clover, kale, lentil, soybean, tobacco, potato, sweet potato, radish, cabbage, rape, apple trees, grape, cotton, sunflower, thale cress, canola, citrus (including orange, mandarin, kumquat, lemon, lime, grapefruit, tangerine, tangelo, citron, and pomelo), pepper, bean, lettuce, Panicum virgatum (switch), Sorghum bicolor (sorghum, sudan), Miscanthus giganteus (miscanthus), Saccharum sp. (energycane), Populus balsamifera (poplar), Zea mays (corn), Glycine max (soybean), Brassica napus (canola), Triticum aestivum (wheat), Gossypium hirsutum (cotton), Oryza sativa (rice), Helianthus annuus (sunflower), Medicago sativa (alfalfa), Beta vulgaris (sugarbeet), Pennisetum glaucum (pearl millet), Panicum spp. Sorghum spp., Miscanthus spp., Saccharum spp., Erianthus spp., Populus spp., Secale cereale (rye), Salix spp. (willow), Eucalyptus spp. (eucalyptus), Triticosecale spp. (triticum-25 wheat X rye), Bamboo, Carthamus tinctorius (safflower), Jatropha curcas (Jatropha), Ricinus communis (castor), Elaeis guineensis (oil palm), Phoenix dactylifera (date palm), Archontophoenix cunninghamiana (king palm), Syagrus romanzoffiana (queen palm), Linum usitatissimum (flax), Brassica juncea, Manihot esculenta (cassaya), Lycopersicon esculentum (tomato), Lactuca saliva (lettuce), Musa paradisiaca (banana), Solanum tuberosum (potato), Brassica oleracea (broccoli, cauliflower, brussel sprouts), Camellia sinensis (tea), Fragaria ananassa (strawberry), Theobroma cacao (cocoa), Coffea arabica (coffee), Vitis vinifera (grape), Ananas comosus (pineapple), Capsicum annum (hot & sweet pepper), Allium cepa (onion), Cucumis melo (melon), Cucumis sativus (cucumber), Cucurbita maxima (squash), Cucurbita moschata (squash), Spinacea oleracea (spinach), Citrullus lanatus (watermelon), Abelmoschus esculentus (okra), Solanum melongena (eggplant), Papaver somniferum (opium poppy), Papaver orientale, Taxus baccata, Taxus brevifolia, Artemisia annua, Cannabis saliva, Camptotheca acuminate, Catharanthus roseus, Vinca rosea, Cinchona officinalis, Coichicum autumnale, Veratrum californica, Digitalis lanata, Digitalis purpurea, Dioscorea 5 spp., Andrographis paniculata, Atropa belladonna, Datura stomonium, Berberis spp., Cephalotaxus spp., Ephedra sinica, Ephedra spp., Erythroxylum coca, Galanthus wornorii, Scopolia spp., Lycopodium serratum (Huperzia serrata), Lycopodium spp., Rauwolfia serpentina, Rauwolfia spp., Sanguinaria canadensis, Hyoscyamus spp., Calendula officinalis, Chrysanthemum parthenium, Coleus forskohlii, Tanacetum parthenium, Parthenium argentatum (guayule), Hevea spp. (rubber), Mentha spicata (mint), Mentha piperita (mint), Bixa orellana, Alstroemeria spp., Rosa spp. (rose), Dianthus caryophyllus (carnation), Petunia spp. (petunia), Poinsettia pulcherrima (Poinsettia), Nicotiana tabacum (tobacco), Lupinus albus (lupin), Uniola paniculata (oats), Hordeum vulgare (barley), and Lolium spp. (rye).

In some examples, a monocotyledonous plant may be used. Monocotyledonous plants belong to the orders of the Alismatales, Arales, Arecales, Bromeliales, Commelinales, Cyclanthales, Cyperales, Eriocaulales, Hydrocharitales, Juncales, Lilliales, Najadales, Orchidales, Pandanales, Poales, Restionales, Triuridales, Typhales, and Zingiberales. Plants belonging to the class of the Gymnospermae are Cycadales, Ginkgoales, Gnetales, and Pinales. In some examples, the monocotyledonous plant can be selected from the group consisting of a maize, rice, wheat, barley, and sugarcane.

In some examples, a dicotyledonous plant may be used, including those belonging to the orders of the Aristochiales, Asterales, Batales, Campanulales, Capparales, Caryophyllales, Casuarinales, Celastrales, Cornales, Diapensales, Dilleniales, Dipsacales, Ebenales, Ericales, Eucomiales, Euphorbiales, Fabales, Fagales, Gentianales, Geraniales, Haloragales, Hamamelidales, Middles, Juglandales, Lamiales, Laurales, Lecythidales, Leitneriales, Magniolales, Malvales, Myricales, Myrtales, Nymphaeales, Papeverales, Piperales, Plantaginales, Plumb aginales, Podostemales, Polemoniales, Polygalales, Polygonales, Primulales, Proteales, Rafflesiales, Ranunculales, Rhamnales, Rosales, Rubiales, Salicales, Santales, Sapindales, Sarraceniaceae, Scrophulariales, Theales, Trochodendrales, Umbellales, Urticales, and Violates. In some examples, the dicotyledonous plant can be selected from the group consisting of cotton, soybean, pepper, and tomato.

In some cases, the plant to be improved is not readily amenable to experimental conditions. For example, a crop plant may take too long to grow enough to practically assess an improved trait serially over multiple iterations. Accordingly, a first plant from which bacteria are initially isolated, and/or the plurality of plants to which genetically manipulated bacteria are applied may be a model plant, such as a plant more amenable to evaluation under desired conditions. Non-limiting examples of model plants include Setaria, Brachypodium, and Arabidopsis. Ability of bacteria isolated according to a method of the disclosure using a model plant may then be applied to a plant of another type (e.g. a crop plant) to confirm conferral of the improved trait.

Traits that may be improved by the methods disclosed herein include any observable characteristic of the plant, including, for example, growth rate, height, weight, color, taste, smell, changes in the production of one or more compounds by the plant (including for example, metabolites, proteins, drugs, carbohydrates, oils, and any other compounds). Selecting plants based on genotypic information is also envisaged (for example, including the pattern of plant gene expression in response to the bacteria, or identifying the presence of genetic markers, such as those associated with increased nitrogen fixation). Plants may also be selected based on the absence, suppression or inhibition of a certain feature or trait (such as an undesirable feature or trait) as opposed to the presence of a certain feature or trait (such as a desirable feature or trait).

### EXAMPLES

The examples provided herein describe methods of bacterial isolation, bacterial and plant analysis, and plant trait improvement. The examples are for illustrative purposes only and are not to be construed as limiting in any way.

### Example 1: Isolation of Microbes from Plant Tissue

Topsoil was obtained from various agricultural areas in central California. Twenty soils with diverse texture characteristics were collected, including heavy clay, peaty clay loam, silty clay, and sandy loam. Seeds of various field corn, sweet corn, heritage corn and tomato were planted into each soil, as shown in Table 1.

TABLE 1
Crop Type and Varieties planted into soil with diverse characteristics

| Crop Type | Field Corn | Sweet Corn | Heritage Corn | Tomato |
|---|---|---|---|---|
| Varieties | Mo17 | Ferry-Morse | Victory Seeds | Ferry-Morse Roma |
| | | ‘Golden Cross | ‘Moseby Prolific’ | VF |
| | | Bantam T-51’ | | |
| | B73 | Ferry-Morse ‘Silver | Victory Seeds ‘Reid's | Stover Roma |
| | | Queen Hybrid’ | Yellow Dent’ | |
| | DKC 66-40 | Ferry-Morse ‘Sugar | Victory Seeds | Totally Tomatoes |
| | | Dots’ | ‘Hickory King’ | ‘Micro Tom Hybrid’ |
| | DKC 67-07 | | | Heinz 1015 |
| | DKC 70-01 | | | Heinz 2401 |
| | | | | Heinz 3402 |
| | | | | Heinz 5508 |
| | | | | Heinz 5608 |
| | | | | Heinz 8504 |

Plants were uprooted after 2-4 weeks of growth and excess soil on root surfaces was removed with deionized water. Following soil removal, plants were surface sterilized with bleach and rinsed vigorously in sterile water. A cleaned, 1 cm section of root was excised from the plant and placed in a phosphate buffered saline solution containing 3 mm steel beads. A slurry was generated by vigorous shaking of the solution with a Qiagen TissueLyser II.

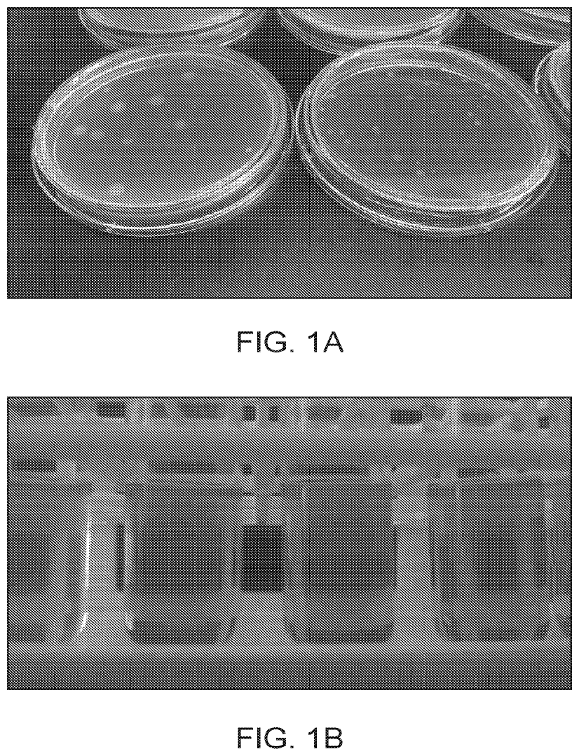

The root and saline slurry was diluted and inoculated onto various types of growth media to isolate rhizospheric, endophytic, epiphytic, and other plant-associated microbes. R2A and Nfb agar media were used to obtain single colonies, and semisolid Nfb media slants were used to obtain populations of nitrogen fixing bacteria. After 2-4 weeks incubation in semi-solid Nfb media slants, microbial populations were collected and streaked to obtain single colonies on R2A agar, as shown in FIG. 1A-B. Single colonies were resuspended in a mixture of R2A and glycerol, subjected to PCR analysis, and frozen at−80° C. for later analysis. Approximately 1,000 single colonies were obtained and designated “isolated microbes.”

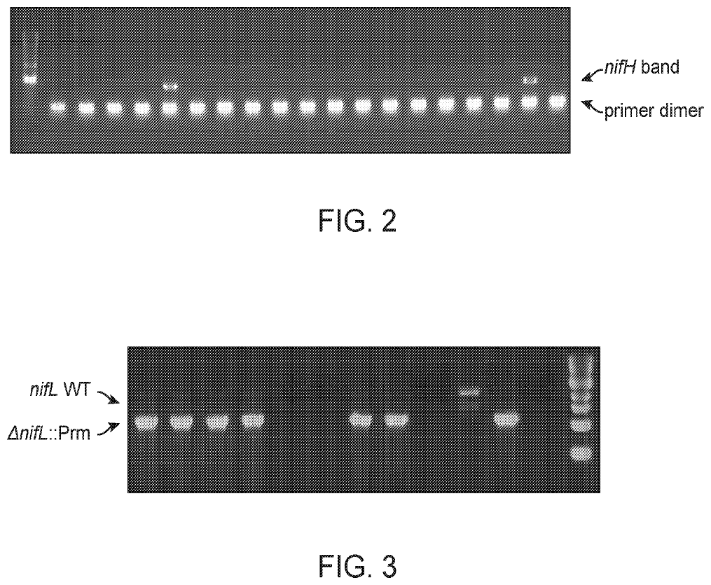

Isolates were then subjected to a colony PCR screen to detect the presence of the nifH gene in order to identify diazotrophs. The previously-described primer set Ueda 19F/388R, which has been shown to detect over 90% of diazotrophs in screens, was used to probe the presence of the nif cluster in each isolate (Ueda et al. 1995; J. Bacteriol. 177: 1414-1417). Single colonies of purified isolates were picked, resuspended in PBS, and used as a template for colony PCR, as shown in FIG. 2. Colonies of isolates that gave positive PCR bands were re-streaked, and the colony PCR and re-streaking process was repeated twice to prevent false positive identification of diazotrophs. Purified isolates were then designated “candidate microbes.”

### Example 2: Characterization of Isolated Microbes

### Sequencing, Analysis and Phylogenetic Characterization

Sequencing of 16S rDNA with the 515f-806r primer set was used to generate preliminary phylogenetic identities for isolated and candidate microbes (see e.g. Vernon et al.; BMC Microbiol. 2002 Dec. 23; 2:39.). The microbes comprise diverse genera including: Enterobacter, Burkholderia, Klebsiella, Bradyrhizobium, Rahnella, Xanthomonas, Raoultella, Pantoea, Pseudomonas, Brevundimonas, Agrobacterium, and Paenibacillus, as shown in Table 2.

TABLE 2
Diversity of microbes isolated from tomato plants
as determined by deep 16S rDNA sequencing.

| Genus | Isolates |
|---|---|
| Achromobacter | 7 |
| Agrobacterium | 117 |
| Agromyces | 1 |
| Alicyclobacillus | 1 |
| Asticcacaulis | 6 |
| Bacillus | 131 |
| Bradyrhizobium | 2 |
| Brevibacillus | 2 |
| Burkholderia | 2 |
| Caulobacter | 17 |
| Chryseobacterium | 42 |
| Comamonas | 1 |
| Dyadobacter | 2 |
| Flavobacterium | 46 |
| Halomonas | 3 |
| Leptothrix | 3 |
| Lysobacter | 2 |
| Neisseria | 13 |
| Paenibacillus | 1 |
| Paenisporosarcina | 3 |
| Pantoea | 14 |
| Pedobacter | 16 |
| Pimelobacter | 2 |
| Pseudomonas | 212 |
| Rhizobium | 4 |
| Rhodoferax | 1 |
| Sphingobacterium | 13 |
| Sphingobium | 23 |
| Sphingomonas | 3 |
| Sphingopyxis | 1 |
| Stenotrophomonas | 59 |
| Streptococcus | 3 |
| Variovorax | 37 |
| Xylanimicrobium | 1 |
| unidentified | 75 |

Subsequently, the genomes of 39 candidate microbes were sequenced using Illumina Miseq platform. Genomic DNA from pure cultures was extracted using the QIAmp DNA mini kit (QIAGEN), and total DNA libraries for sequencing were prepared through a third party vendor (SeqMatic, Hayward). Genome assembly was then carried out via the A5 pipeline (Tritt et al. 2012; PLoS One 7(9):e42304). Genes were identified and annotated, and those related to regulation and expression of nitrogen fixation were noted as targets for mutagenesis.

### Transcriptomic Profiling of Candidate Microbes

Transcriptomic profiling of strain CI010 was performed to identify promoters that are active in the presence of environmental nitrogen. Strain CI010 was cultured in a defined, nitrogen-free media supplemented with 10 mM glutamine. Total RNA was extracted from these cultures (QIAGEN RNeasy kit) and subjected to RNAseq sequencing via Illumina HiSeq (SeqMatic, Fremont CA). Sequencing reads were mapped to CI010 genome data using Geneious, and highly expressed genes under control of proximal transcriptional promoters were identified. Tables 3A-C lists genes and their relative expression level as measured through RNASeq sequencing of total RNA. Sequences of the proximal promoters were recorded for use in mutagenesis of nif pathways, nitrogen utilization related pathways, or other genes with a desired expression level.

### Assessment of Genetic Tractability

Candidate microbes were characterized based on transformability and genetic tractability. First, optimal carbon source utilization was determined by growth on a small panel of relevant media as well as a growth curve in both nitrogen-free and rich media. Second, the natural antibiotic resistance of each strain was determined through spot-plating and growth in liquid culture containing a panel of antibiotics used as selective markers for mutagenesis. Third, each strain was tested for its transformability through electroporation of a collection of plasmids. The plasmid collection comprises the combinatorial expansion of seven origins of replication, i.e., p15a, pSC101, CloDF, colA, RK2, pBBR1, and pRO1600 and four antibiotic resistance markers, i.e., CmR, KmR, SpecR, and TetR. This systematic evaluation of origin and resistance marker compatibility was used to identify vectors for plasmid-based mutagenesis in candidate microbes.

### Example 3: Mutagenesis of Candidate Microbes

### Lambda-Red Mediated Knockouts

Several mutants of candidate microbes were generated using the plasmid pKD46 or a derivative containing a kanamycin resistance marker (Datsenko et al. 2000; PNAS 97(12): 6640-6645). Knockout cassettes were designed with 250 bp homology flanking the target gene and generated via overlap extension PCR. Candidate microbes were transformed with pKD46, cultured in the presence of arabinose to induce Lambda-Red machinery expression, prepped for electroporation, and transformed with the knockout cassettes to produce candidate mutant strains. Four candidate microbes and one laboratory strain, Klebsiella oxytoca M5A1, were used to generate thirteen candidate mutants of the nitrogen fixation regulatory genes nifL, glnB, and amtB, as shown in Table 4.

TABLE 4
List of single knockout mutants created through Lambda-red
mutagenesis Oligo-Directed Mutagenesis with Cas9 Selection

| Strain | nifL | glnB | amtB |
|---|---|---|---|
| M5A1 | X | X | X |
| CI006 | X | X | X |
| CI010 | X | X | X |
| CI019 | X | X | |
| CI028 | X | X | |

Oligo-directed mutagenesis was used to target genomic changes to the rpoB gene in E. coli DH10B, and mutants were selected with a CRISPR-Cas system. A mutagenic oligo (ss1283: “G*T*T*G*ATCAGACCGATGTTCGGACCTTCcaagGTTTCGATCGGACATACGCGAC CGTAGTGGGTCGGGTGTACGTCTCGAACTTCAAAGCC”, where * denotes phosphorothioate bond) was designed to confer rifampicin resistance through a 4-bp mutation to the rpoB gene. Cells containing a plasmid encoding Cas9 were induced for Cas9 expression, prepped for electroporation, and then electroporated with both the mutagenic oligo and a plasmid encoding constitutive expression of a guide RNA (gRNA) that targets Cas9 cleavage of the WT rpoB sequence. Electroporated cells were recovered in nonselective media overnight to allow sufficient segregation of the resulting mutant chromosomes. After plating on selection for the gRNA-encoding plasmid, two out of ten colonies screened were shown to contain the desired mutation, while the rest were shown to be escape mutants generated through protospacer mutation in the gRNA plasmid or Cas9 plasmid loss.

Lambda-Red Mutagenesis with Cas9 Selection

Mutants of candidate microbes CI006 and CIO10 were generated via lambda-red mutagenesis with selection by CRISPR-Cas. Knockout cassettes contained an endogenous promoter identified through transcriptional profiling (as described in Example 2 and depicted in Table 3) and ˜250 bp homology regions flanking the deletion target. CI006 and CI010 were transformed with plasmids encoding the Lambda-red recombination system (exo, beta, gam genes) under control of an arabinose inducible promoter and Cas9 under control of an IPTG inducible promoter. The Red recombination and Cas9 systems were induced in resulting transformants, and strains were prepared for electroporation. Knockout cassettes and a plasmid-encoded selection gRNA were subsequently transformed into the competent cells. After plating on antibiotics selective for both the Cas9 plasmid and the gRNA plasmid, 7 of the 10 colonies screened showed the intended knockout mutation, as shown in FIG. 3.

### Example 4: In Vitro Phenotyping of Candidate Molecules

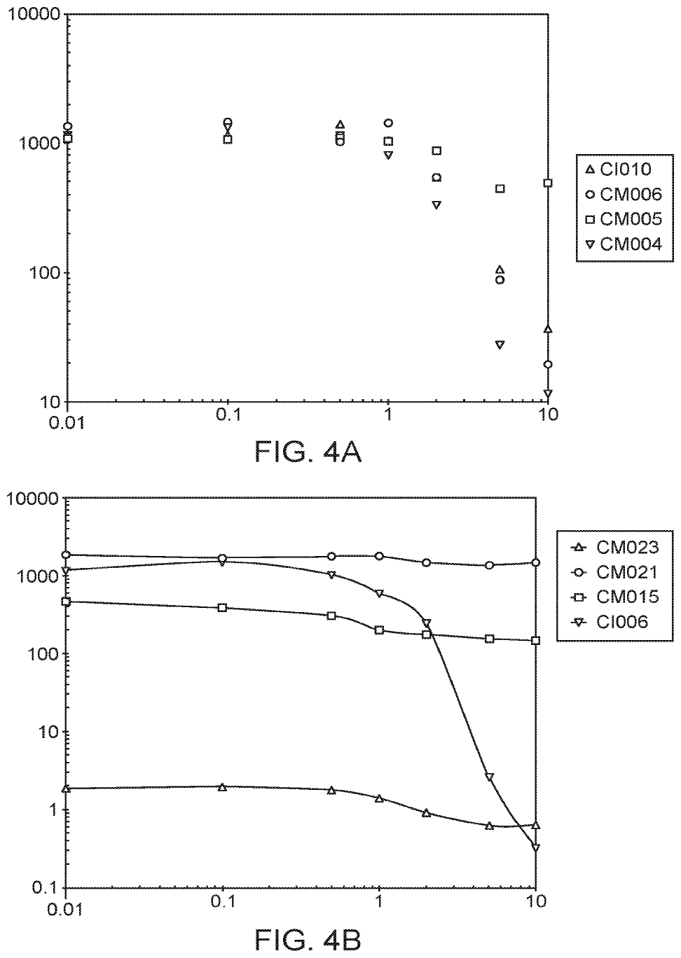

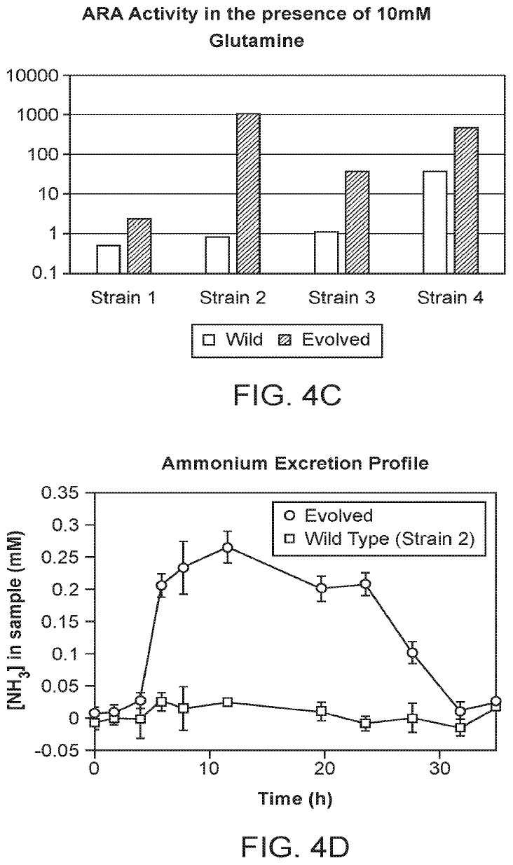

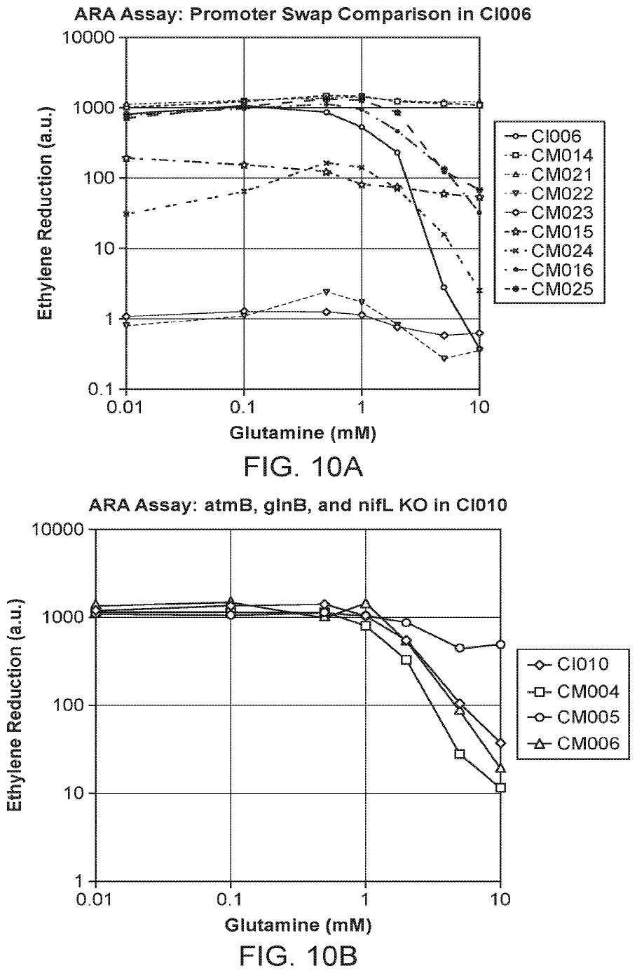

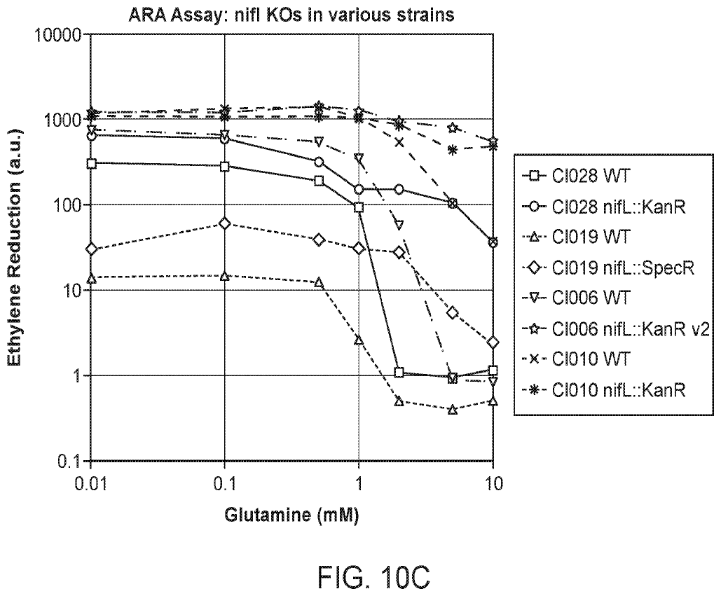

The impact of exogenous nitrogen on nitrogenase biosynthesis and activity in various mutants was assessed. The Acetylene Reduction Assay (ARA) (Temme et. al. 2012; 109(18): 7085-7090) was used to measure nitrogenase activity in pure culture conditions. Strains were grown in air-tight test tubes, and reduction of acetylene to ethylene was quantified with an Agilent 6890 gas chromatograph. ARA activities of candidate microbes and counterpart candidate mutants grown in nitrogen fixation media supplemented with 0 to 10 mM glutamine are shown in FIGS. 4A-B and FIGS. 10A-C.

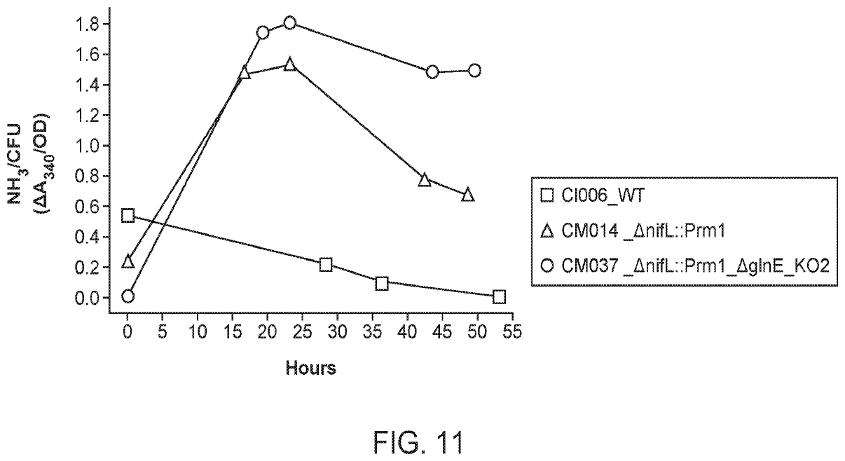

Under anaerobic culture conditions, a range of glutamine and ammonia concentrations was tested to quantify impact on nitrogen fixation activity. In wild-type cells, activity quickly diminished as glutamine concentrations increased. However, in a series of initial knock-out mutations, a class of mutation was validated enabling expression of nitrogen fixation genes under concentrations of glutamine that would otherwise shut off activity in wild type. This profile was generated in four different species of diazotrophs, as seen in FIG. 4C. In addition, by rewiring the regulatory network using genetic parts that have been identified, the nitrogen fixation activity level was tuned predictably. This is seen in FIG. 4B, which illustrates strains CM023, CM021, CM015, and C1006. Strain CM023 is an evolved strain low; strain CM021 is an evolved strain high; strain CM015 is an evolved strain mid; strain CI006 is a wild-type (strain 2). Ammonia excreted into culture supernatants was tested using a enzymatic-based assay (MEGAZYME). The assay measures the amount of NADPH consumed in the absorbance of 340 nm. The assay was conducted on bacterial cultures grown in nitrogen-free, anaerobic environment with a starting density of 1E9 CFU/ml. Across a panel of six evolved strains, one strain excreted up to 100 μM of ammonia over a course of a 48 hour period, as seen in FIG. 4D. Further, a double mutant exhibited higher ammonia excretion than the single mutant from which it was derived, as seen in FIG. 11. This demonstrates a microbial capacity to produce ammonia in excess of its physiological needs.

### Transcription Profiling of Pure Cultures

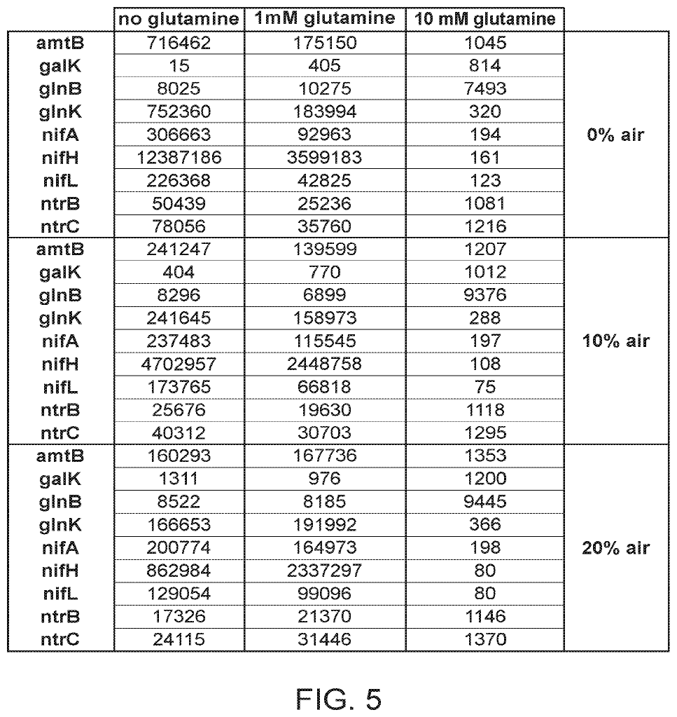

Transcriptional activity of CI006 was measured using the Nanostring Elements platform. Cells were grown in nitrogen-free media and 10E8 cells were collected after 4 hours incubation. Total RNA was extracted using the Qiagen RNeasy kit. Purified RNA was submitted to Core Diagnostics in Palo Alto, CA, for probe hybridization and Digital Analyzer analysis, as shown in FIG. 5.

### Example 5: In Planta Phenotyning of Candidate Microbes

### Colonization of Plants by Candidate Microbes

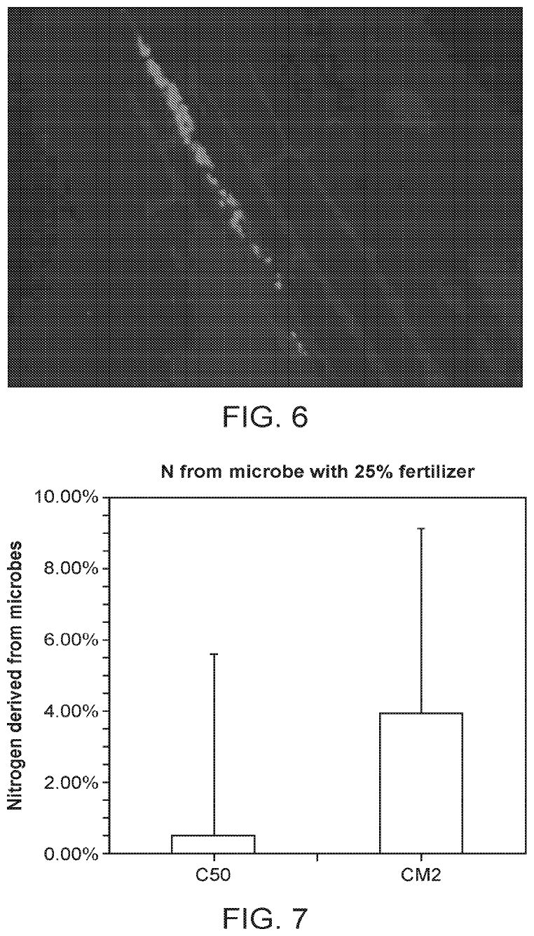

Colonization of desired host plants by a candidate microbe was quantified through short-term plant growth experiments. Corn plants were inoculated with strains expressing RFP either from a plasmid or from a Tn5-integrated RFP expression cassette. Plants were grown in both sterilized sand and nonsterile peat medium, and inoculation was performed by pipetting 1 mL of cell culture directly over the emerging plant coleoptile three days post-germination. Plasmids were maintained by watering plants with a solution containing the appropriate antibiotic. After three weeks, plant roots were collected, rinsed three times in sterile water to remove visible soil, and split into two samples. One root sample was analyzed via fluorescence microscopy to identify localization patterns of candidate microbes. Microscopy was performed on 10 mm lengths of the finest intact plant roots, as shown in FIG. 6.

A second quantitative method for assessing colonization was developed. A quantitative PCR assay was performed on whole DNA preparations from the roots of plants inoculated with the endophytes. Seeds of corn (Dekalb DKC-66-40) were germinated in previously autoclaved sand in a 2.5 inch by 2.5 inch by 10 inch pot. One day after planting, 1 ml of endophyte overnight culture (SOB media) was drenched right at the spot of where the seed was located. 1 mL of this overnight culture is roughly equivalent to about 10 \hat{ }9 cfu, varying within 3-fold of each other, depending on which strain is being used. Each seedling was fertilized 3× weekly with 50 mL modified Hoagland's solution supplemented with either 2.5 mM or 0.25 mM ammonium nitrate. At four weeks after planting, root samples were collected for DNA extraction. Soil debris were washed away using pressurized water spray. These tissue samples were then homogenized using QIAGEN Tissuelyzer and the DNA was then extracted using QIAmp DNA Mini Kit (QIAGEN) according to the recommended protocol. qPCR assay was performed using Stratagene Mx3005P RT-PCR on these DNA extracts using primers that were designed (using NCBI's Primer BLAST) to be specific to a loci in each of the endophyte's genome. The presence of the genome copies of the endophytes was quantified. To further confirm the identity of the endophytes, the PCR amplification products were sequenced and are confirmed to have the correct sequence. The summary of the colonization profile of strain CI006 and CI008 from candidate microbes are presented in Table 5. Colonization rate as high as 10\hat{ }7× cfu/g fw of root was demonstrated in strain C1008.

TABLE 5
Colonization of corn as measured by qPCR

| Strain | Colonization Rate (CFU/g fw) |
|---|---|
| CI006 | 1.45 × 10\hat{ }5 |
| CI008 | 1.24 × 10\hat{ }7 |

### In Planta RNA Profiling

Biosynthesis of nif pathway components in planta was estimated by measuring the transcription of nif genes. Total RNA was obtained from root plant tissue of CI006 inoculated plants (planting methods as described previously). RNA extraction was performed using RNEasy Mini Kit according to the recommended protocol (QIAGEN). Total RNA from these plant tissues was then assayed using Nanostring Elements kits (NanoString Technologies, Inc.) using probes that were specific to the nif genes in the genome of strain CI006. The data of nif gene expression in planta is summarized in Table 6. Expression of nifH genes was detected in plants inoculated by CM013 strains whereas nifH expression was not detectable in CI006 inoculated plants. Strain CM013 is a derivative of strain CI006 in which the nifL gene has been knocked out.

Highly expressed genes of CM011, ranked by transcripts per kilobase million (TPM), were measured in planta under fertilized condition. The promoters controlling expression of some of these highly expressed genes were used as templates for homologous recombination into targeted nitrogen fixation and assimilation loci. RNA samples from greenhouse grown CM011 inoculated plant were extracted, rRNA removed using Ribo-Zero kit, sequenced using Illumina's Truseq platform and mapped back to the genome of CM011. Highly expressed genes from CM011 are listed in Table 7.

TABLE 6
Expression of nifH in planta

| Strains | Relative Transcript Expression |
|---|---|
| CI006 | 9.4 |
| CM013 | 103.25 |

TABLE 7

| | | | | TPM |
|---|---|---|---|---|
| | | | | (Transcripts |
| | | | Raw | Per |
| | Gene | | Read | Kilobase |
| Gene Name | Location | Direction | Count | Million) |
| rpsH CDS | 18196-18588 | reverse | 4841.5 | 27206.4 |
| rplQ CDS | 11650-12039 | reverse | 4333 | 24536.2 |
| rpsJ CDS | 25013-25324 | reverse | 3423 | 24229 |
| rplV CDS | 21946-22278 | reverse | 3367.5 | 22333 |
| rpsN CDS | 18622-18927 | reverse | 2792 | 20150.1 |
| rplN CDS | 19820-20191 | reverse | 3317 | 19691.8 |
| rplF CDS | 17649-18182 | reverse | 4504.5 | 18628.9 |
| rpsD CDS | 13095-13715 | reverse | 5091.5 | 18106.6 |
| rpmF CDS | 8326-8493 | forward | 1363.5 | 17923.8 |
| rplW CDS | 23429-23731 | reverse | 2252 | 16413.8 |
| rpsM CDS | 14153-14509 | reverse | 2269 | 14036.2 |
| rplR CDS | 17286-17639 | reverse | 2243.5 | 13996.1 |
| rplC CDS | 24350-24979 | reverse | 3985 | 13969.2 |
| rplK CDS | 25526-25954 | reverse | 2648.5 | 13634.1 |
| rplP CDS | 20807-21217 | reverse | 2423 | 13019.5 |
| rplX CDS | 19495-19809 | reverse | 1824 | 12787.8 |
| rpsQ CDS | 20362-20616 | reverse | 1460.5 | 12648.7 |
| bhsA 3 CDS | 79720-79977 | reverse | 1464 | 12531.5 |
| rpmC CDS | 20616-20807 | reverse | 998.5 | 11485 |
| rpoA CDS | 12080-13069 | reverse | 4855 | 10830.2 |
| rplD CDS | 23728-24333 | reverse | 2916.5 | 10628.5 |
| bhsA 1 CDS | 78883-79140 | reverse | 1068 | 9141.9 |
| rpsS CDS | 22293-22571 | reverse | 1138.5 | 9011.8 |
| rpmA CDS | 2210-2467 | forward | 1028.5 | 8803.7 |
| rpmD CDS | 16585-16764 | reverse | 694.5 | 8520.8 |
| rplB CDS | 22586-23410 | reverse | 3132 | 8384 |
| rpsC CDS | 21230-21928 | reverse | 2574.5 | 8133.9 |
| rplE CDS | 18941-19480 | reverse | 1972.5 | 8066.9 |
| rplO CDS | 16147-16581 | reverse | 1551 | 7874.2 |
| preprotein translocase | 14808-16139 | reverse | 4657 | 7721.2 |
| subunit SecY CDS | | | | |
| rpsE CDS | 16771-17271 | reverse | 1671.5 | 7368 |
| rpsK CDS | 13746-14135 | reverse | 1223.5 | 6928.2 |
| tufA CDS | 27318-28229 | reverse | 2850 | 6901.3 |
| rpmI CDS | 38574-38771 | forward | 615 | 6859.5 |
| rplU CDS | 1880-2191 | forward | 935.5 | 6621.7 |
| rplT CDS | 38814-39170 | forward | 1045 | 6464.4 |
| bhsA 2 CDS | 79293-79550 | reverse | 754 | 6454.1 |
| rpmB CDS | 8391-8627 | reverse | 682 | 6355.1 |
| rplJ CDS | 23983-24480 | reverse | 1408 | 6243.9 |
| fusA 2 CDS | 481-2595 | reverse | 5832 | 6089.6 |
| rpsA CDS | 25062-26771 | reverse | 4613 | 5957.6 |
| rpmJ CDS | 14658-14774 | reverse | 314 | 5926.9 |
| rpsR CDS | 52990-53217 | forward | 603 | 5840.7 |
| rpsG CDS | 2692-3162 | reverse | 1243 | 5828.2 |
| rpsI CDS | 11354-11746 | reverse | 980.5 | 5509.8 |
| cspC 1 CDS | 8091-8300 | reverse | 509 | 5352.8 |
| rpsF CDS | 52270-52662 | forward | 916 | 5147.4 |
| rpsT CDS | 55208-55471 | reverse | 602 | 5035.9 |
| infC CDS | 38128-38478 | forward | 755 | 4750.3 |
| cspG CDS | 30148-30360 | forward | 446 | 4624.2 |

### ¹⁵N Assay

The primary method for demonstrating fixation uses the nitrogen isotope 15N, which is found in the atmosphere at a set rate relative to 14N. By supplementing either fertilizer or atmosphere with enriched levels of 15N, one can observe fixation either directly, in heightened amounts of 15N fixed from an atmosphere supplemented with 15N₂ gas (Yoshida 1980), or inversely, through dilution of enriched fertilizer by atmospheric N₂ gas in plant tissues (Iniguez 2004). The dilution method allows for the observation of cumulative fixed nitrogen over the course of plant growth, while the 15N₂ gas method is restricted to measuring the fixation that occurs over the short interval that a plant can be grown in a contained atmosphere (rate measurement). Therefore, the gas method is superior in specificity (as any elevated 15N₂ levels in the plant above the atmospheric rate can be attributed unambiguously to fixation) but cannot show cumulative activity.

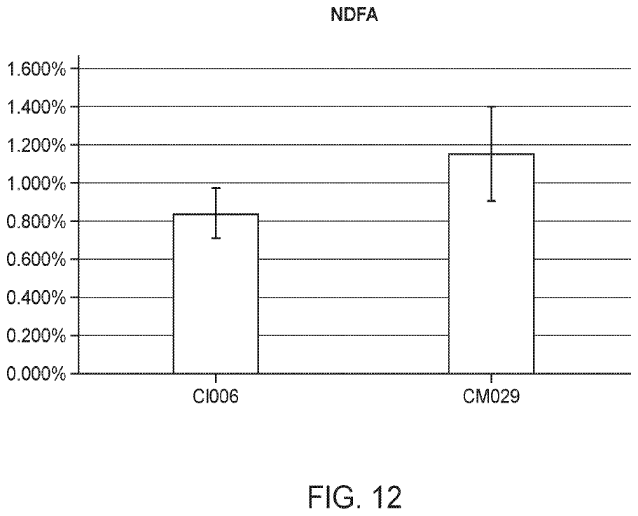

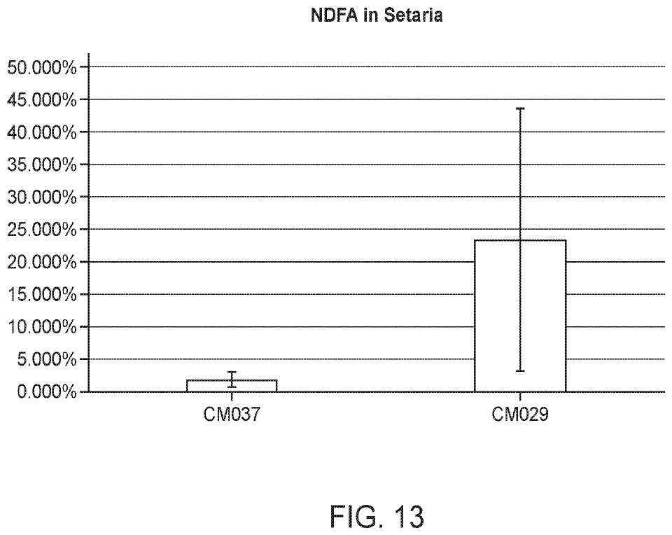

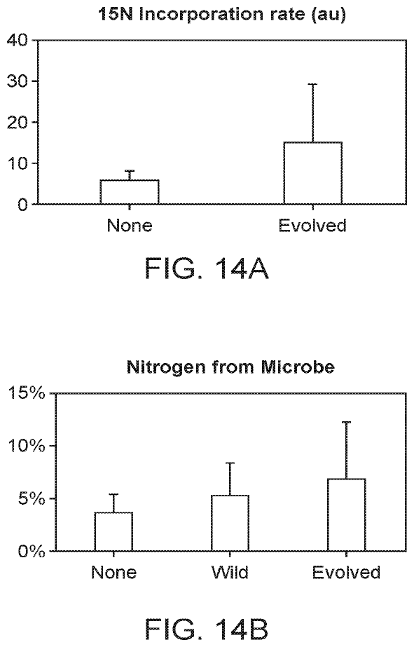

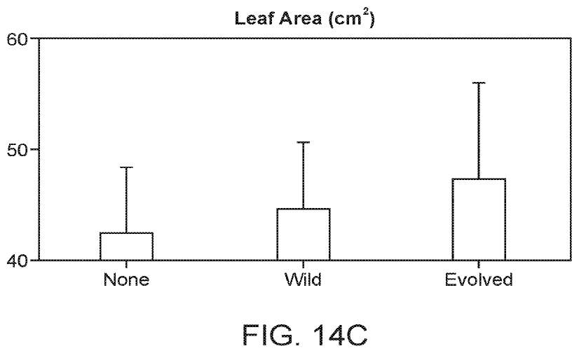

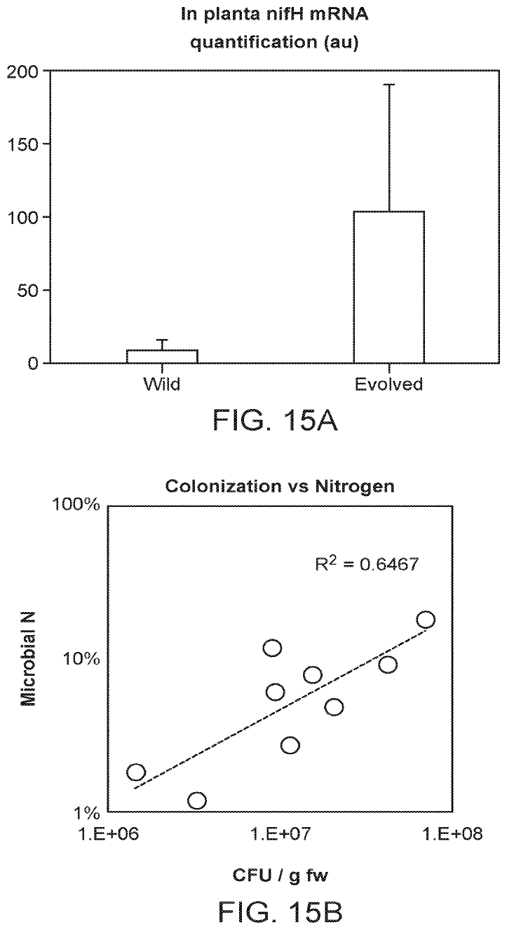

Both types of assay has been performed to measure fixation activity of improved strains relative to wild-type and uninoculated corn plants, and elevated fixation rates were observed in planta for several of the improved strains (FIG. 12, FIG. 14A, and FIG. 14B). These assays are instrumental in demonstrating that the activity of the strains observed in vitro translates to in vivo results. Furthermore, these assays allow measurement of the impact of fertilizer on strain activity, suggesting suitable functionality in an agricultural setting. Similar results were observed when setaria plants were inoculated with wild-type and improved strains (FIG. 13). In planta fixation activity shown in FIGS. 14A-14C is further backed up by transcriptomic data. Evolved strains exhibit increased nifH transcript level relative to wild-type counterparts. Furthermore, the microbe derived nitrogen level in planta is also correlated with the colonization level on a plant by plant basis. These results (FIG. 12, FIG. 13, FIGS. 14A-14C, FIG. 15A, and FIG. 15B) support the hypothesis that the microbe, through the improved regulation of the nif gene cluster, is the likely reason for the increase in atmospheric derived nitrogen seen in the plant tissue. In addition to measuring fixation directly, the impact of inoculating plants with the improved strains in a nitrogen-stressed plant biomass assay was measured. While plant biomass may be related to many possible microbe interactions with the plant, one would expect that the addition of fixed nitrogen would impact the plant phenotype when nitrogen is limited. Inoculated plants were grown in the complete absence of nitrogen, and significant increases in leaf area, shoot fresh and dry weight, and root fresh and dry weight in inoculated plants relative to untreated controls was observed (FIG. 14C). Although these differences cannot be attributed to nitrogen fixation exclusively, they support the conclusion that the improved strains are actively providing nitrogen to the plant. Corn and setaria plants were grown and inoculated as described above. Fertilizer comprising 1.2% ¹⁵N was regularly supplied to plants via watering. Nitrogen fixation by microbes was quantified by measuring the ¹⁵N level in the plant tissue. Fourth leaf tissue was collected and dried at 4 weeks after planting. Dried leaf samples were homogenized using beads (QIAGEN Tissuelyzer) and aliquoted out into tin capsules for IRMS (MBL Stable Isotope Laboratory at The Ecosystems Center, Woods Hole, MA). Nitrogen derived from the atmosphere (NDFA) was calculated, and nitrogen production by CI050 and CM002 are shown in FIG. 7.

### Phytohormone Production Assay

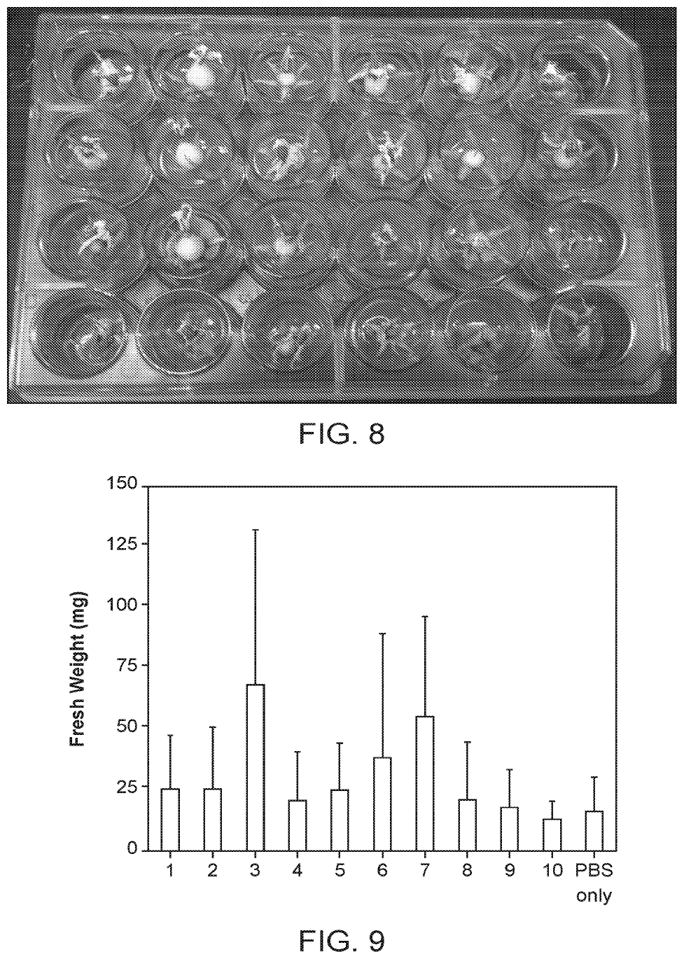

The dwarf tomato (Solanum lycopersicum) cultivar ‘Micro-Tom’ has previously been used to study the influence of indole-3-acetic acid on fruit ripening through an in vitro assay (Cohen 1996; J Am Soc Hortic Sci 121: 520-524). To evaluate phytohormone production and secretion by candidate microbes, a plate-based screening assay using immature Micro-Tom fruit was developed. Twelve-well tissue culture test plates were prepared by filling wells with agar medium, allowing it to solidify, and spotting 10 uL of overnight microbial cultures onto the agar surface, as shown in FIG. 8. Wells with agar containing increasing amounts of gibberellic acid (GA) but no bacterial culture were used as a positive control and standards. Flowers one day post-anthesis abscised from growing Micro-Tom plants were inserted, stem-first, into the agar at the point of the bacterial spot culture. These flowers were monitored for 2-3 weeks, after which the fruits were harvested and weighed. An increase in plant fruit mass across several replicates indicates production of plant hormone by the inoculant microbe, as shown in FIG. 9.

### Example 6: Cyclical Host-Microbe Evolution

Corn plants were inoculated with CM013 and grown 4 weeks to approximately the V5 growth stage. Those demonstrating improved nitrogen accumulation from microbial sources via ¹⁵N analysis were uprooted, and roots were washed using pressurized water to remove bulk soil. A 0.25 g section of root was cut and rinsed in PBS solution to remove fine soil particles and non-adherent microbes. Tissue samples were homogenized using 3 mm steel beads in QIAGEN TissueLyser II. The homogenate was diluted and plated on SOB agar media. Single colonies were resuspended in liquid media and subjected to PCR analysis of 16s rDNA and mutations unique to the inoculating strain. The process of microbe isolation, mutagenesis, inoculation, and re-isolation can be repeated iteratively to improve microbial traits, plant traits, and the colonization capability of the microbe.

### Example 7: Compatibility Across Geography

The ability of the improved microbes to colonize an inoculated plant is critical to the success of the plant under field conditions. While the described isolation methods are designed to select from soil microbes that may have a close relationship with crop plants such as corn, many strains may not colonize effectively across a range of plant genotypes, environments, soil types, or inoculation conditions. Since colonization is a complex process requiring a range of interactions between a microbial strain and host plant, screening for colonization competence has become a central method for selecting priority strains for further development. Early efforts to assess colonization used fluorescent tagging of strains, which was effective but time-consuming and not scalable on a per-strain basis. As colonization activity is not amenable to straightforward improvement, it is imperative that potential product candidates are selected from strains that are natural colonizers.

An assay was designed to test for robust colonization of the wild-type strains in any given host plant using qPCR and primers designed to be strain-specific in a community sample. This assay is intended to rapidly measure the colonization rate of the microbes from corn tissue samples. Initial tests using strains assessed as probable colonizers using fluorescence microscopy and plate-based techniques indicated that a qPCR approach would be both quantitative and scalable.

A typical assay is performed as follows: Plants, mostly varieties of maize and wheat, are grown in a peat potting mix in the greenhouse in replicates of six per strain. At four or five days after planting, a 1 mL drench of early stationary phase cultures of bacteria diluted to an OD590 of 0.6-1.0 (approximately 5E+08 CFU/mL) is pipetted over the emerging coleoptile. The plants are watered with tap water only and allowed to grow for four weeks before sampling, at which time, the plants are uprooted and the roots washed thoroughly to remove most peat residues. Samples of clean root are excised and homogenized to create a slurry of plant cell debris and associated bacterial cells. We developed a high-throughput DNA extraction protocol that effectively produced a mixture of plant and bacterial DNA to use as template for qPCR. Based on bacterial cell spike-in experiments, this DNA extraction process provides a quantitative bacterial DNA sample relative to the fresh weight of the roots. Each strain is assessed using strain-specific primers designed using Primer BLAST (Ye 2012) and compared to background amplification from uninoculated plants. Since some primers exhibit off-target amplification in uninoculated plants, colonization is determined either by presence of amplification or elevated amplification of the correct product compared to the background level.

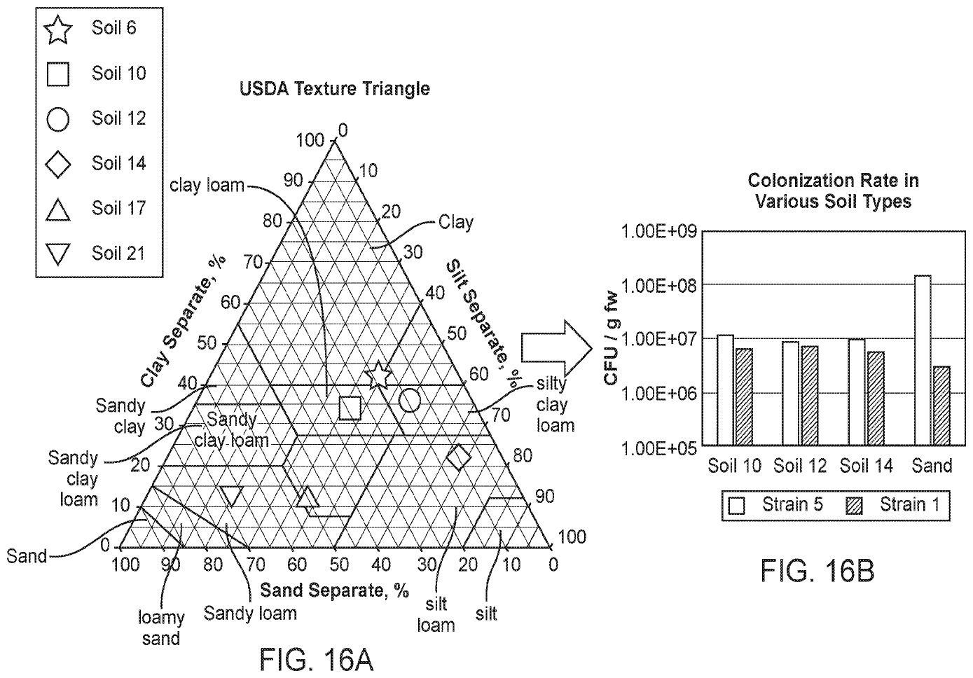

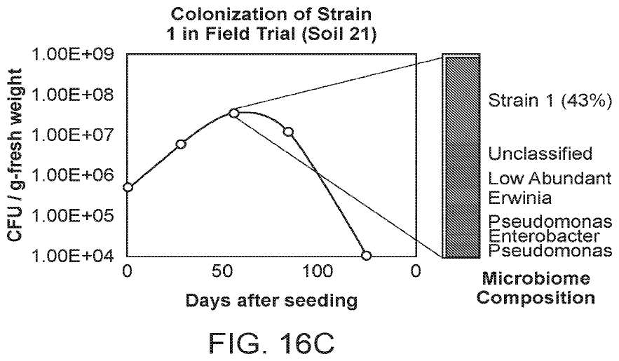

This assay was used to measure the compatibility of the microbial product across different soil geography. Field soil qualities and field conditions can have a huge influence on the effect of a microbial product. Soil pH, water retention capacity, and competitive microbes are only a few examples of factors in soil that can affect inoculum survival and colonization ability. A colonization assay was performed using three diverse soil types sampled from agricultural fields in California as the plant growth medium (FIG. 16A). An intermediate inoculation density was used to approximate realistic agricultural conditions. Within 3 weeks, Strain 5 colonized all plants at 1E+06 to 1E+07 CFU/g FW. After 7 weeks of plant growth, an evolved version of Strain 1 exhibited high colonization rates (1E+06 CFU/g FW) in all soil types. (FIG. 16B).

Additionally, to assess colonization in the complexity of field conditions, a 1-acre field trial in in San Luis Obispo in June of 2015 was initiated to assess the impacts and colonization of seven of the wild-type strains in two varieties of field corn. Agronomic design and execution of the trial was performed by a contract field research organization, Pacific Ag Research. For inoculation, the same peat culture seed coating technique tested in the inoculation methods experiment was employed. During the course of the growing season, plant samples were collected to assess for colonization in the root and stem interior. Samples were collected from three replicate plots of each treatment at four and eight weeks after planting, and from all six reps of each treatment shortly before harvest at 16 weeks. Additional samples were collected from all six replicate plots of treatments inoculated with Strain 1 and Strain 2, as well as untreated controls, at 12 weeks. Numbers of cells per gram fresh weight of washed roots were assessed as with other colonization assays with qPCR and strain-specific primers. Two strains, Strain 1 and Strain 2, showed consistent and widespread root colonization that peaked at 12 weeks and then declined precipitously (FIG. 16C). While Strain 2 appeared to be present in numbers an order of magnitude lower than Strain 1, it was found in more consistent numbers from plant to plant. No strains appeared to effectively colonize the stem interior. In support of the qPCR colonization data, both strains were successfully re-isolated from the root samples using plating and 16S sequencing to identify isolates of matching sequence

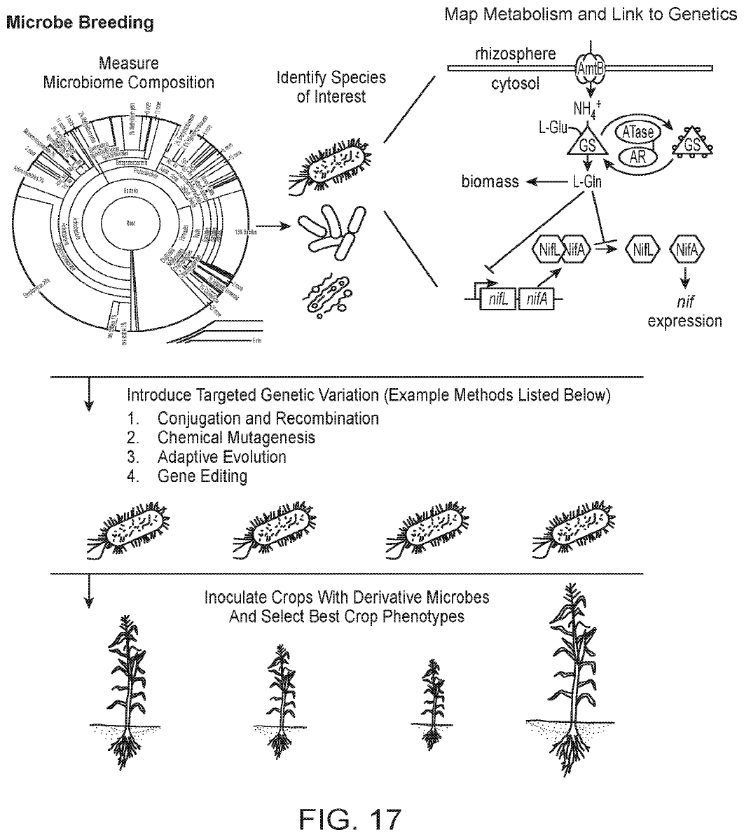

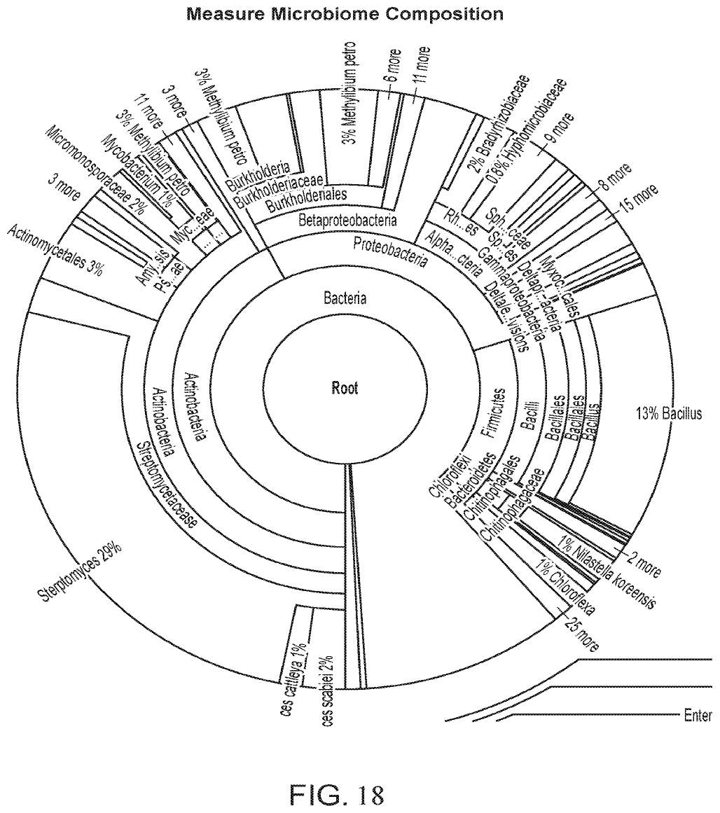

Examples of microbe breeding can be summarized in the schematic of FIG. 17 and FIG. 18. FIG. 17 depicts microbe breeding wherein the composition of the microbiome can be first measured and a species of interest is identified. The metabolism of the microbiome can be mapped and linked to genetics. Afterwards, a targeted genetic variation can be introduced using methods including, but not limited to, conjugation and recombination, chemical mutagenesis, adaptive evolution, and gene editing. Derivative microbes are used to inoculate crops. In some examples, the crops with the best phenotypes are selected.

As provided in FIG. 17, the composition of the microbiome can be first measured and a species of interest is identified. The metabolism of the microbiome can be mapped and linked to genetics. The metabolism of nitrogen can involve the entrance of ammonia (NH₄⁺) from the rhizosphere into the cytosol of the bacteria via the AmtB transporter. Ammonia and L-glutamate (L-Glu) are catalyzed by glutamine synthetase and ATP into glutamine. Glutamine can lead to the formation of biomass (plant growth), and it can also inhibit expression of the nif operon. Afterwards, a targeted genetic variation can be introduced using methods including, but not limited to, conjugation and recombination, chemical mutagenesis, adaptive evolution, and gene editing. Derivative microbes are used to inoculate crops. The crops with the best phenotypes are selected.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. For example, if the range 10-15 is disclosed, then 11, 12, 13, and 14 are also disclosed. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

### Example 8 Promoter Validation

For each selected promoter, a strain was generated in which the nifL gene was deleted and replaced with the promoter inserted upstream of the nifA gene, which is a transcriptional activator of nitrogenase expression in the absence of nifL. Each of these mutants was tested in an acetylene reduction assay, in which anaerobic cultures are exposed to acetylene gas, which the nitrogenase enzyme reduces to ethylene gas, which can be detected via gas chromatography. The rate of acetylene reduction corresponds to the amount of nitrogenase present in the sample, and thus served as a readout for nifA transcription. In some cases, samples of from the acetylene reduction assay were subjected to RNA extraction, and transcription of the nifA and nitrogenase genes were measured via qPCR. The results are shown in Table 6.

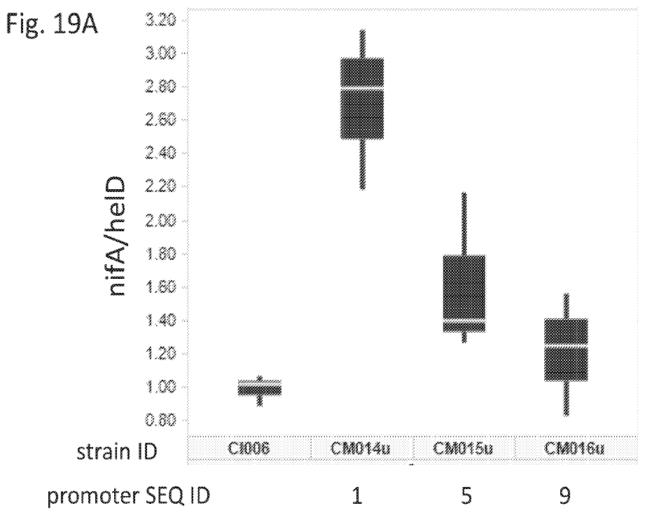

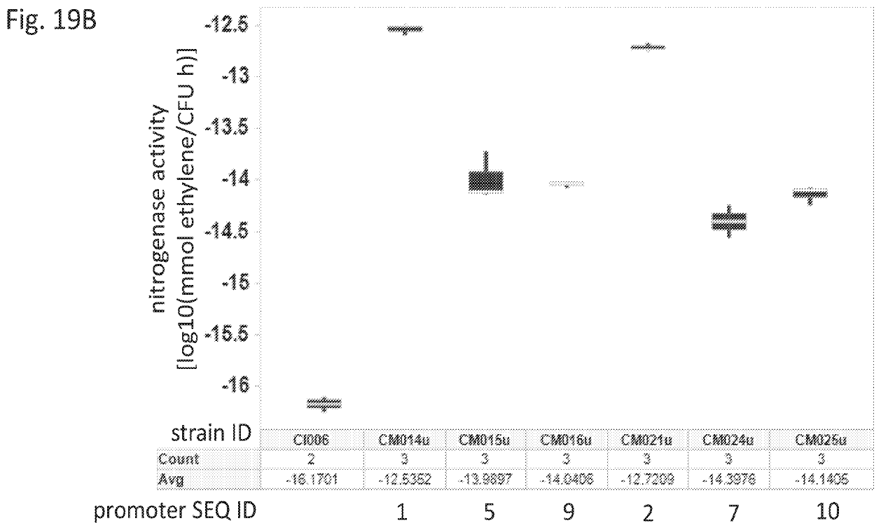

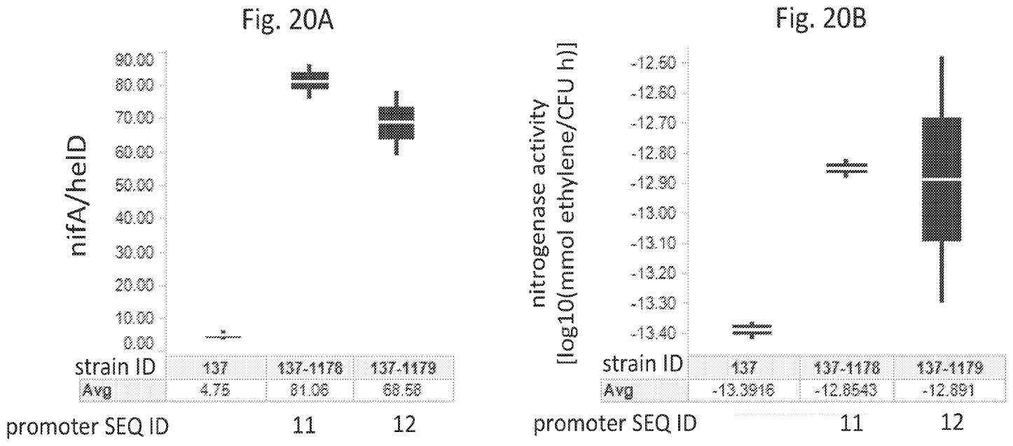

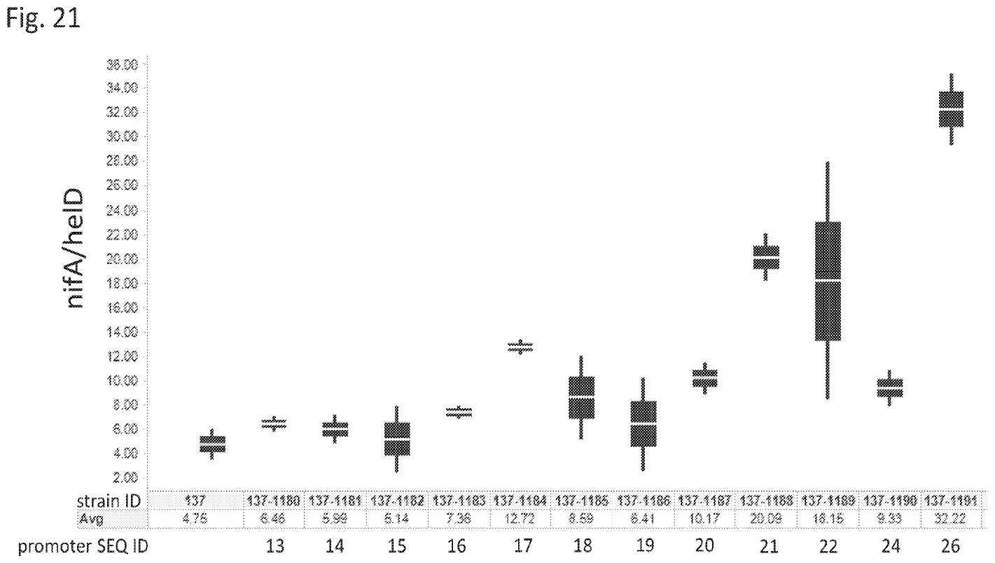

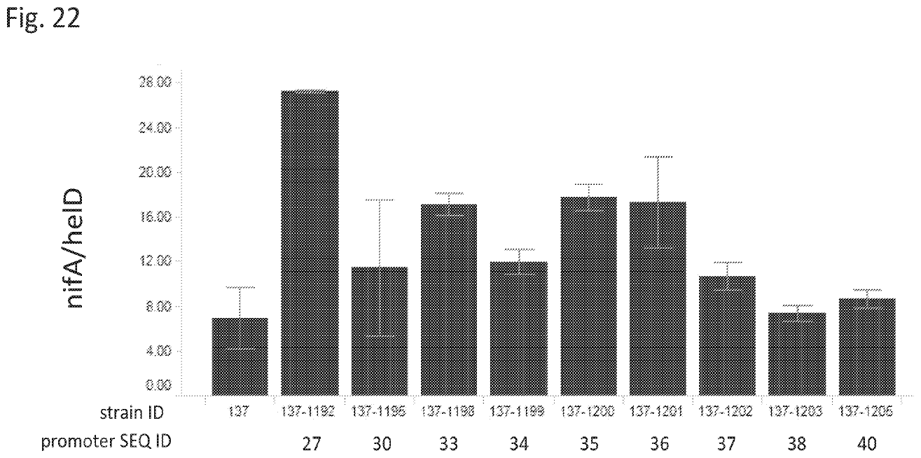

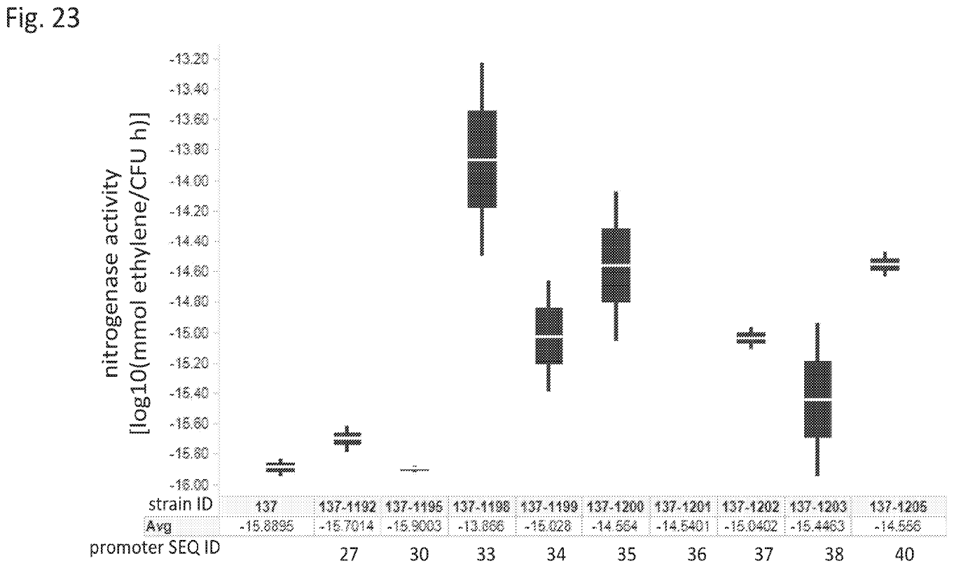

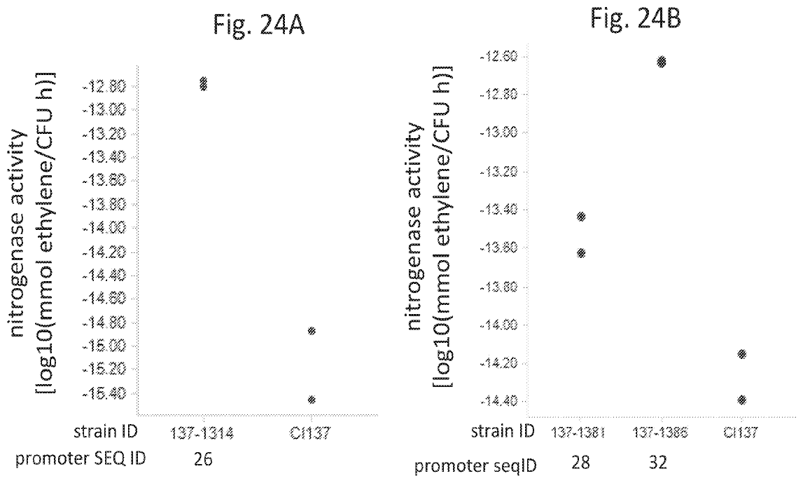

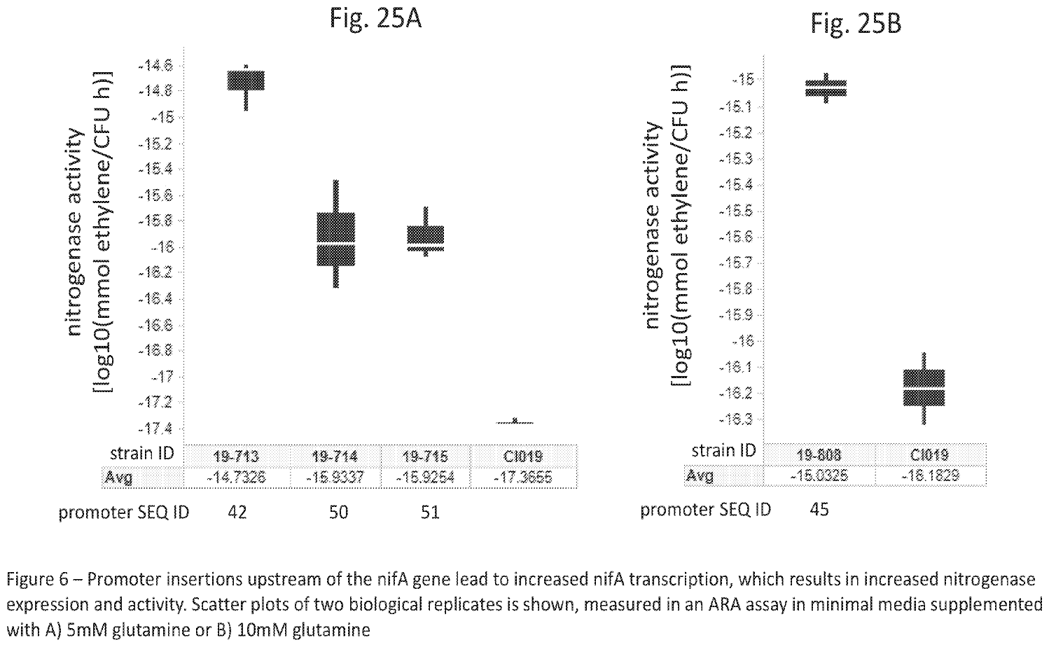

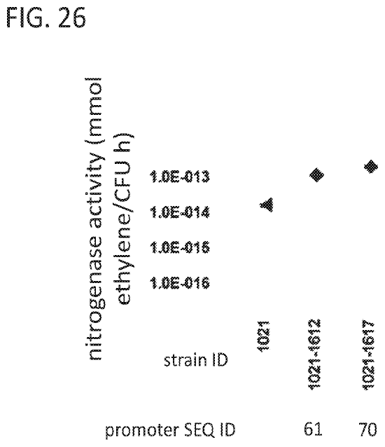

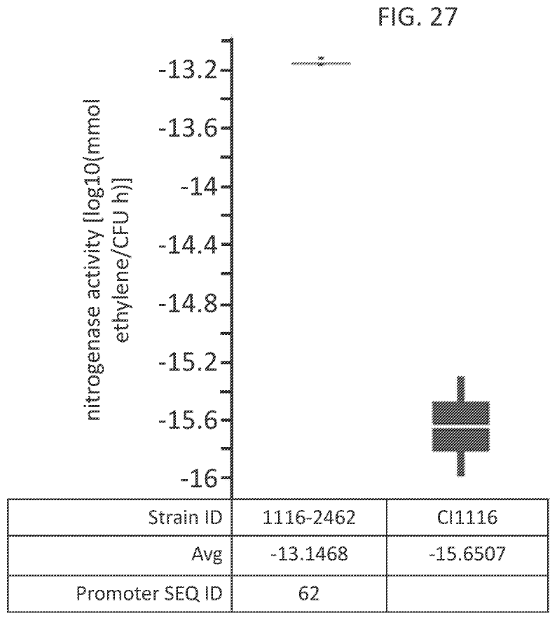

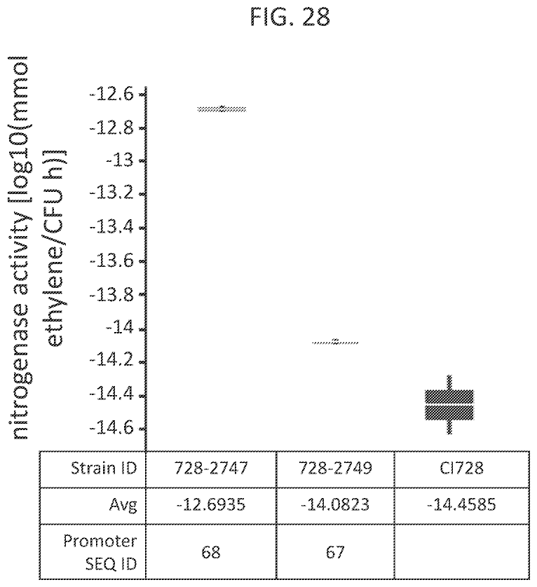

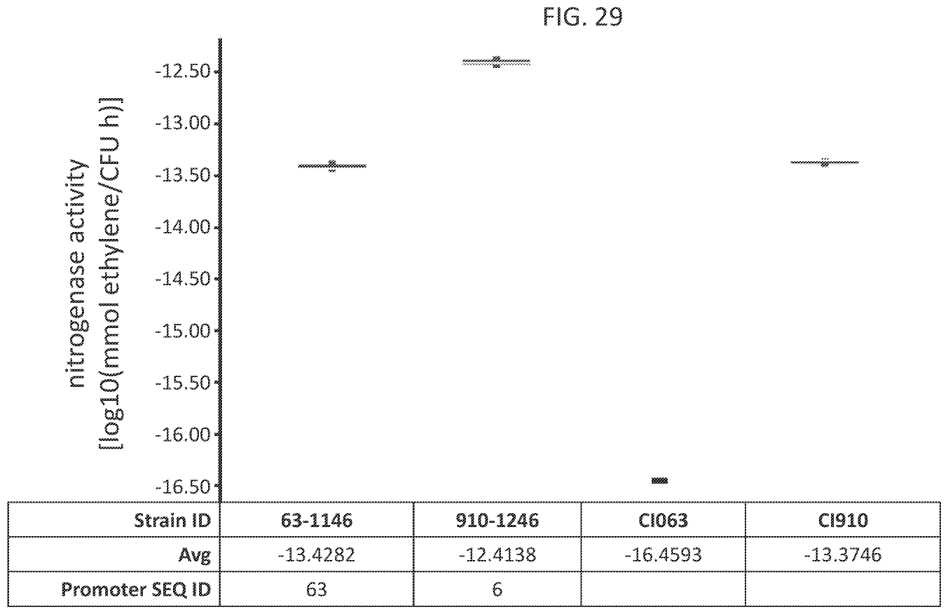

Further results are shown in FIGS. 19-31C. In FIGS. 19A, 19B, and 24A—the NifL gene has been deleted and replaced with a heterologous promoter—as indicated in the figures. No DNA sequences exogenous to the host cell were introduced into the genome by this process. As shown in FIGS. 19A, 20A, 21, and 22, nifA expression is increased compared to wildtype levels when nifA is operably linked to any of SEQ ID NOs.: 1, 5, 9, 11-24, 26, 27, 30, 33-37, and 40. As shown in FIGS. 19B, 20B, 23, 24A, 24B, 25A, 25B, 26, 27, 28, and 29 nitrogenase activity is increased, as compared to wildtype levels, when nifA is operably linked to any of SEQ ID NOs.: 1, 2, 5-7, 9-12, 26-28, 32-38, 40, 42, 45, 50-51, 61-63, 67, and 70. Genetically engineered strains depicted in FIGS. 24A, 24B, 25B, and 26-29 were cured to remove all non-native promoter sequences. Genetically engineered strains depicted in FIGS. 19A-23, 25A, 30 and 31 were not cured prior to assaying.

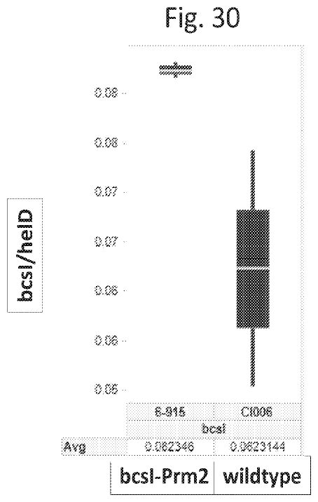

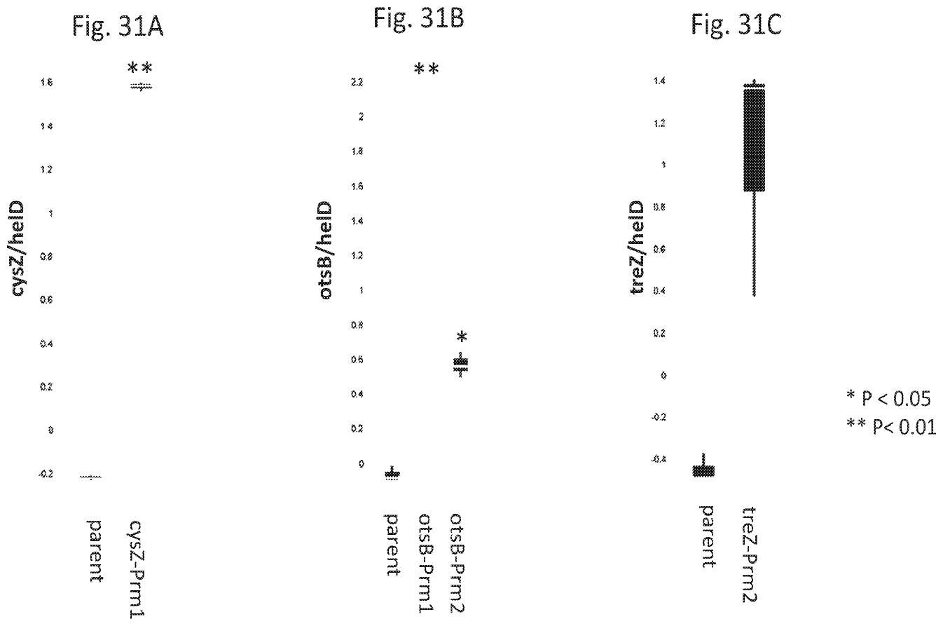

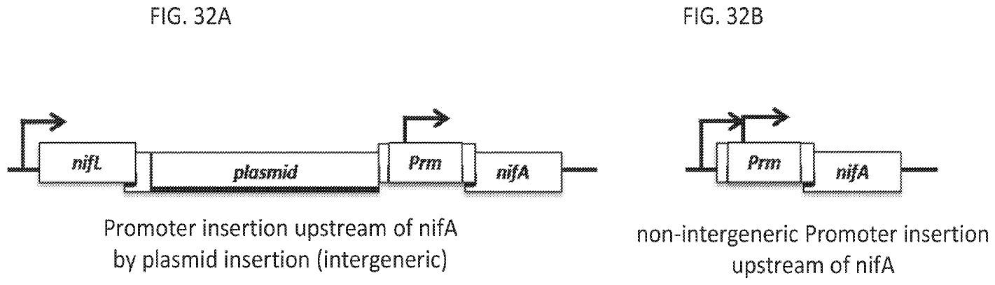

FIGS. 30-31C show that heterologous promoters of the present disclosure can also be used to upregulate other genes beyond NifA. In FIGS. 30-31C native promoter sequences of bcsI, otsB, CysZ, or treZ were replaced with heterologous sequences of this disclosure. As shown in FIG. 30 expression of bcsI was increased when the gene was operably linked to SEQ ID NO.: 2. As shown in FIG. 31A expression of CysZ was increased when the gene was operably linked to SEQ ID NO.: 1. As shown in FIG. 31B expression of otsB was increased when the gene was operably linked to either SEQ ID NO.: 1 or SEQ ID NO.: 2. Finally, as shown in FIG. 31C expression of treZ was increased when the gene was operably linked to SEQ ID NO.: 2.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

TABLE 3A

| Name | Minimum | Maximum | Length | Direction |
|---|---|---|---|---|
| murein lipoprotein CDS | 2,929,898 | 2,930,134 | 237 | forward |
| membrane protein CDS | 5,217,517 | 5,217,843 | 327 | forward |
| zinc/cadmium-binding | 3,479,979 | 3,480,626 | 648 | forward |
| protein CDS | | | | |
| acyl carrier protein CDS | 4,563,344 | 4,563,580 | 237 | reverse |
| ompX CDS | 4,251,002 | 4,251,514 | 513 | forward |
| DNA-binding protein HU- | 375,156 | 375,428 | 273 | forward |
| beta CDS | | | | |
| sspA CDS | 629,998 | 630,636 | 639 | reverse |
| tatE CDS | 3,199,435 | 3,199,638 | 204 | reverse |
| LexA repressor CDS | 1,850,457 | 1,851,065 | 609 | forward |
| hisS CDS | <3999979 | 4,001,223 | >1245 | forward |

TABLE 3B

| | Differential | | | | | |
|---|---|---|---|---|---|---|
| | Expression | Differential | RNASeq_nifL - | RNASeq_nifL - | RNASeq_WT - | RNASeq_WT - |
| | Absolute | Expression | Raw Read | Raw Transcript | Raw Read | Raw Transcript |
| Name | Confidence | Ratio | Count | Count | Count | Count |
| murein | 1000 | −1.8 | 12950.5 | 10078.9 | 5151.5 | 4106.8 |
| lipoprotein | | | | | | |
| CDS | | | | | | |
| membrane | 1000 | −1.3 | 9522.5 | 5371.3 | 5400 | 3120 |
| protein CDS | | | | | | |
| zinc/cadmium- | 3.3 | 1.1 | 6461 | 1839.1 | 5318 | 1550.6 |
| binding protein | | | | | | |
| CDS | | | | | | |
| acyl carrier | 25.6 | 1.6 | 1230.5 | 957.6 | 1473.5 | 1174.7 |
| protein CDS | | | | | | |
| ompX CDS | 1.7 | 1.1 | 2042 | 734.2 | 1687.5 | 621.5 |
| DNA-binding | 6.9 | −1.3 | 1305 | 881.7 | 725 | 501.8 |
| protein HU- | | | | | | |
| beta CDS | | | | | | |
| sspA CDS | 0.2 | 1 | 654 | 188.8 | 504.5 | 149.2 |
| tatE CDS | 1.4 | 1.3 | 131 | 118.4 | 125 | 115.8 |
| LexA | 0.1 | −1.1 | 248 | 75.1 | 164 | 50.9 |
| repressor CDS | | | | | | |
| hisS CDS | 0 | −1.1 | 467 | 69.2 | 325 | 49.3 |

TABLE 4
Table of Strains

| | First | Current | Universal | | | |
|---|---|---|---|---|---|---|
| Sort | Reference | Name | Name | Lineage | Mutagenic DNA Description | Genotype |
| 1 | Application | CI006 | CI006 | Isolated strain from | None | WT |
| | text | | | Enterobacter genera | | |
| 2 | Application | CI008 | CI008 | Isolated strain from | None | WT |
| | text | | | Burkholderia genera | | |
| 3 | Application | CI010 | CI010 | Isolated strain from | None | WT |
| | text | | | Klebsiella genera | | |
| 4 | Application | CI019 | CI019 | Isolated strain from | None | WT |
| | text | | | Rahnella genera | | |
| 5 | Application | CI028 | CI028 | Isolated strain from | None | WT |
| | text | | | Enterobacter genera | | |
| 6 | Application | CI050 | CI050 | Isolated strain from | None | WT |
| | text | | | Klebsiella genera | | |
| 7 | Application | CM002 | CM002 | Mutant of CI050 | Disruption of nifL gene with a | ΔnifL::KanR |
| | text | | | | kanamycin resistance | |
| | | | | | expression cassette (KanR) | |
| | | | | | encoding the aminoglycoside O- | |
| | | | | | phosphotransferase gene aphl | |
| | | | | | inserted. | |
| 8 | Application | CM011 | CM011 | Mutant of CI019 | Disruption of nifL gene with a | ΔnifL::SpecR |
| | text | | | | spectinomycin resistance | |
| | | | | | expression cassette (SpecR) | |
| | | | | | encoding the streptomycin 3″- | |
| | | | | | O-adenylyltransferase gene | |
| | | | | | aadA inserted. | |
| 9 | Application | CM013 | CM013 | Mutant of CI006 | Disruption of nifL gene with a | ΔnifL::KanR |
| | text | | | | kanamycin resistance | |
| | | | | | expression cassette (KanR) | |
| | | | | | encoding the aminoglycoside O- | |
| | | | | | phosphotransferase gene aphl | |
| | | | | | inserted. | |
| 10 | FIG. 4A | CM004 | CM004 | Mutant of CI010 | Disruption of amtB gene with a | ΔamtB::KanR |
| | | | | | kanamycin resistance | |
| | | | | | expression cassette (KanR) | |
| | | | | | encoding the aminoglycoside O- | |
| | | | | | phosphotransferase gene aphl | |
| | | | | | inserted. | |
| 11 | FIG. 4A | CM005 | CM005 | Mutant of CI010 | Disruption of nifL gene with a | ΔnifL::KanR |
| | | | | | kanamycin resistance | |
| | | | | | expression cassette (KanR) | |
| | | | | | encoding the aminoglycoside O- | |
| | | | | | phosphotransferase gene aphl | |
| | | | | | inserted. | |
| 12 | FIG. 4B | CM015 | CM015 | Mutant of CI006 | Disruption of nifL gene with a | ΔnifL::Prm5 |
| | | | | | fragment of the region upstream | |
| | | | | | of the ompX gene inserted | |
| | | | | | (Prm5). | |
| 13 | FIG. 4B | CM021 | CM021 | Mutant of CI006 | Disruption of nifL gene with a | ΔnifL::Prm2 |
| | | | | | fragment of the region upstream | |
| | | | | | of an unanotated gene and the | |
| | | | | | first 73 bp of that gene inserted | |
| | | | | | (Prm2). | |
| 14 | FIG. 4B | CM023 | CM023 | Mutant of CI006 | Disruption of nifL gene with a | ΔnifL::Prm4 |
| | | | | | fragment of the region upstream | |
| | | | | | of the acpP gene and the first | |
| | | | | | 121 bp of the acpP gene inserted | |
| | | | | | (Prm4). | |
| 15 | FIG. 10A | CM014 | CM014 | Mutant of CI006 | Disruption of nifL gene with a | ΔnifL::Prm1 |
| | | | | | fragment of the region upstream | |
| | | | | | of the 1 pp gene and the first | |
| | | | | | 29 bp of the 1 pp gene inserted | |
| | | | | | (Prm1). | |
| 16 | FIG. 10A | CM016 | CM016 | Mutant of CI006 | Disruption of nifL gene with a | ΔnifL::Prm9 |
| | | | | | fragment of the region upstream | |
| | | | | | of the lexA 3 gene and the first | |
| | | | | | 2 lbp of the lexA 3 gene | |
| | | | | | inserted (Prm9). | |
| 17 | FIG. 10A | CM022 | CM022 | Mutant of CI006 | Disruption of nifL gene with a | ΔnifL::Prm3 |
| | | | | | fragment of the region upstream | |
| | | | | | of the mntP 1 gene and the first | |
| | | | | | 53 bp of the mntP 1 gene | |
| | | | | | inserted (Prm3). | |
| 18 | FIG. 10A | CM024 | CM024 | Mutant of CI006 | Disruption of nifL gene with a | ΔnifL::Prm7 |
| | | | | | fragment of the region upstream | |
| | | | | | of the sspA gene inserted | |
| | | | | | (Prm7). | |
| 19 | FIG. 10A | CM025 | CM025 | Mutant of CI006 | Disruption of nifL gene with a | ΔnifL::Prm10 |
| | | | | | fragment of the region upstream | |
| | | | | | of the hisS gene and the first | |
| | | | | | 52 bp of the hisS gene inserted | |
| | | | | | (Prm10). | |
| 20 | FIG. 10B | CM006 | CM006 | Mutant of CI010 | Disruption of glnB gene with a | ΔglnB::KanR |
| | | | | | kanamycin resistance | |
| | | | | | expression cassette (KanR) | |
| | | | | | encoding the aminoglycoside O- | |
| | | | | | phosphotransferase gene aphl | |
| | | | | | inserted. | |
| 21 | FIG. 10C | CI028 | CM017 | Mutant of CI028 | Disruption of nifL gene with a | ΔnifL::KanR |
| | | nifL:KanR | | | kanamycin resistance | |
| | | | | | expression cassette (KanR) | |
| | | | | | encoding the aminoglycoside O- | |
| | | | | | phosphotransferase gene aphl | |
| | | | | | inserted. | |
| 22 | FIG. 10C | CI019 | CM011 | Mutant of CI019 | Disruption of nifL gene with a | ΔnifL::SpecR |
| | | nifL:SpecR | | | spectinomycin resistance | |
| | | | | | expression cassette (SpecR) | |
| | | | | | encoding the streptomycin 3″- | |
| | | | | | O-adenylyltransferase gene | |
| | | | | | aadA inserted. | |
| 23 | FIG. 10C | CI006 | CM013 | Mutant of CI006 | Disruption of nifL gene with a | ΔnifL::KanR |
| | | nifL:KanR | | | kanamycin resistance | |
| | | | | | expression cassette (KanR) | |
| | | | | | encoding the aminoglycoside O- | |
| | | | | | phosphotransferase gene aphl | |
| | | | | | inserted. | |
| 24 | FIG. 10C | CI10 | CM005 | Mutant of CI010 | Disruption of nifL gene with a | ΔnifL::KanR |
| | | nifL:KanR | | | kanamycin resistance | |
| | | | | | expression cassette (KanR) | |
| | | | | | encoding the aminoglycoside O- | |
| | | | | | phosphotransferase gene aphl | |
| | | | | | inserted. | |
| 25 | FIG. 4C | Strain 2 | CI006 | Isolated strain from | None | WT |
| | | | | Enterobacter genera | | |
| 26 | FIG. 4C | Strain 4 | CI010 | Isolated strain from | None | WT |
| | | | | Klebsiella genera | | |
| 27 | FIG. 4C | Strain 1 | CI019 | Isolated strain from | None | WT |
| | | | | Rahnella genera | | |
| 28 | FIG. 4C | Strain 3 | CI028 | Isolated strain from | None | WT |
| | | | | Enterobacter genera | | |
| 29 | FIG. 4B | Strain 2 | CI006 | Isolated strain from | None | WT |
| | | | | Enterobacter genera | | |
| 30 | FIG. 4B | High | CM014 | Mutant of CI006 | Disruption of nifL gene with a | ΔnifL::Prml |
| | | | | | fragment of the region upstream | |
| | | | | | of the 1 pp gene and the first | |
| | | | | | 29 bp of the 1 pp gene inserted | |
| | | | | | (Prm1). | |
| 31 | FIG. 4B | Med | CM015 | Mutant of CI006 | Disruption of nifL gene with a | ΔnifL::Prm5 |
| | | | | | fragment of the region upstream | |
| | | | | | of the ompX gene inserted | |
| | | | | | (Prm5). | |
| 32 | FIG. 4B | Low | CM023 | Mutant of CI006 | Disruption of nifL gene with a | ΔnifL::Prm4 |
| | | | | | fragment of the region upstream | |
| | | | | | of the acpP gene and the first | |
| | | | | | 121 bp of the acpP gene inserted | |
| | | | | | (Prm4). | |
| 33 | FIG. 4D | Strain 2 | CI006 | Isolated strain from | None | WT |
| | | | | Enterobacter genera | | |
| 34 | FIG. 4D | Evolved | CM029 | Mutant of CI006 | Disruption of nifL gene with a | ΔnifL::Prm5 |
| | | | | | fragment of the region upstream | ΔglnE- |
| | | | | | of the ompX gene inserted | AR_KO1 |
| | | | | | (Prm5) and deletion of the | |
| | | | | | 1287 bp after the start codon of | |
| | | | | | the glnE gene containing the | |
| | | | | | adenylyl-removing domain of | |
| | | | | | glutamate-ammonia-ligase | |
| | | | | | adenylyltransferase (AglnE- | |
| | | | | | AR_KOI). | |
| 35 | FIG. 14C | Wild | CI006 | Isolated strain from | None | WT |
| | | | | Enterobacter genera | | |
| 36 | FIG. 14C | Evolved | CM014 | Mutant of CI006 | Disruption of nifL gene with a | ΔnifL::Prm1 |
| | | | | | fragment of the region upstream | |
| | | | | | of the 1 pp gene and the first | |
| | | | | | 29 bp of the 1 pp gene inserted | |
| | | | | | (Prm1). | |
| 37 | FIG. 14B | Wild | CI019 | Isolated strain from | None | WT |
| | | | | Rahnella genera | | |
| 38 | FIG. 14B | Evolved | CM011 | Mutant of CI019 | Disruption of nifL gene with a | ΔnifL::SpecR |
| | | | | | spectinomycin resistance | |
| | | | | | expression cassette (SpecR) | |
| | | | | | encoding the streptomycin 3″- | |
| | | | | | O-adenylyltransferase gene | |
| | | | | | aadA inserted. | |
| 39 | FIG. 14A | Evolved | CM011 | Mutant of CI019 | Disruption of nifL gene with a | ΔnifL::SpecR |
| | | | | | spectinomycin resistance | |
| | | | | | expression cassette (SpecR) | |
| | | | | | encoding the streptomycin 3″- | |
| | | | | | O-adenylyltransferase gene | |
| | | | | | aadA inserted. | |
| 40 | FIG. 15A | Wild | CI006 | Isolated strain from | None | WT |
| | | | | Enterobacter genera | | |
| 41 | FIG. 15A | Evolved | CM013 | Mutant of CI006 | Disruption of nifL gene with a | ΔnifL::KanR |
| | | | | | kanamycin resistance | |
| | | | | | expression cassette (KanR) | |
| | | | | | encoding the aminoglycoside O- | |
| | | | | | phosphotransferase gene aph1 | |
| | | | | | inserted. | |
| 42 | FIG. 15B | No name | CM011 | Mutant of CI019 | Disruption of nifL gene with a | ΔnifL::SpecR |
| | | | | | spectinomycin resistance | |
| | | | | | expression cassette (SpecR) | |
| | | | | | encoding the streptomycin 3″- | |
| | | | | | O-adenylyltransferase gene | |
| | | | | | aadA inserted. | |
| 43 | FIG. 16B | Strain 5 | CI008 | Isolated strain from | None | WT |
| | | | | Burkholderia genera | | |
| 44 | FIG. 16B | Strain 1 | CM011 | Mutant of CI019 | Disruption of nifL gene with a | ΔnifL::SpecR |
| | | | | | spectinomycin resistance | |
| | | | | | expression cassette (SpecR) | |
| | | | | | encoding the streptomycin 3″- | |
| | | | | | O-adenylyltransferase gene | |
| | | | | | aadA inserted. | |

| | First | Current | Universal | | | | Gene 2 |
|---|---|---|---|---|---|---|---|
| Sort | Reference | Name | Name | Lineage | Mutagenic DNA Description | Genotype | mutation |
| 34 | FIG. 4D | Evolved | CM029 | Mutant of | Disruption of nifL gene with a | ΔnifL::Prm5 | |
| | | | | CI006 | fragment of the region upstream of | ΔglnE- | |
| | | | | | the ompX gene inserted (Prm5) and | AR_KO1 | |
| | | | | | deletion of the 1287 bp after the | | |
| | | | | | start codon of the glnE gene | | |
| | | | | | containing the adenylyl-removing | | |
| | | | | | domain of glutamate-ammonia- ligase | | |
| | | | | | adenylyltransferase | | |
| | | | | | (ΔglnE-AR_KO1). | | |

TABLE 8
Promoter sequences

| SEQ ID | |
|---|---|
| NO. | Sequence |
| 1 | CGTCCTGTAATAATAACCGGACAATTCGGACTGATTAAAAAAGCGCCCTTGTGGCGCTTTTT |
| | TTATATTCCCGCCTCCATTTAAAATAAAAAATCCAATCGGATTTCACTATTTAAACTGGCCAT |
| | TATCTAAGATGAATCCGATGGAAGCTCGCTGTTTTAACACGCGTTTTTTAACCTTTTATTGAA |
| | AGTCGGTGCTTCTTTGAGCGAACGATCAAATTTAAGTGGATTCCCATCAAAAAAATATTCTC |
| | AACCTAAAAAAGTTTGTGTAATACTTGTAACGCTACATGGAGATTAACTCAATCTAGAGGGT |
| | ATTAATAATGAATCGTACTAAACTGGTACTGGGCGC |
| 2 | TCACCACGGCGATAACCATAGGTTTTCGGCGTGGCCACATCCATGGTGAATCCCACTTTTTCC |
| | AGCACGCGCGCCACTTCATCGGGTCTTAAATACATAGATTTTCCTCGTCATCTTTCCAAAGCC |
| | TCGCCACCTTACATGACTGAGCATGGACCGTGACTCAGAAAATTCCACAAACGAACCTGAAA |
| | GGCGTGATTGCCGTCTGGCCTTAAAAATTATGGTCTAAACTAAAATTTACATCGAAAACGAG |
| | GGAGGATCCTATGTTTAACAAACCGAATCGCCGTGACGTAGATGAAGGTGTTGAGGATATTA |
| | ACCACGATGTTAACCAGCTCG |
| 3 | ATCATATTGCGCTCCCTGGTTATCATTTGTTACTAAATGAAATGTTATAATATAACAATTATA |
| | AATACCACATCGCTTTCAATTCACCAGCCAAATGAGAGGAGCGCCGTCTGACATAGCCAGCG |
| | CTATAAAACATAGCATTATCTATATGTTTATGATTAATAACTGATTTTTGCGTTTTGGATTTG |
| | GCTGTGGCATCCTTGCCGCTCTTTTCGCAGCGTCTGCGTTTTTGCCCTCCGGTCAGGGCATTT |
| | AAGGGTCAGCAATGAGTTTTTACGCAATTACGATTCTTGCCTTCGGCATGTCGATGGATGCTTT |
| 4 | TGACGAGGCAGGTTACATCACTGGTGAAACCCTGCACGTCAATGGCGGAATGTATATGGTTT |
| | AACCACGATGAAAATTATTTGCGTTATTAGGGCGAAAGGCCTCAAAATAGCGTAAAATCGTG |
| | GTAAGAACTGCCGGGATTTAGTTGCAAATTTTTCAACATTTTATACACTACGAAAACCATCG |
| | CGAAAGCGAGTTTTGATAGGAAATTTAAGAGTATGAGCACTATCGAAGAACGCGTTAAGAA |
| | AATTATCGGCGAACAGCTGGGCGTTAAGCAGGAAGAAGTTACCAACAATGCTTCCTTCGTTG |
| | AAGACCTGGGCGCTGATTCTCTTGACACCG |
| 5 | GGACATCATCGCGACAAACAATATTAATACCGGCAACCACACCGGCAATTTACGAGACTGC |
| | GCAGGCATCCTTTCTCCCGTCAATTTCTGTCAAATAAAGTAAAAGAGGCAGTCTACTTGAAT |
| | TACCCCCGGCTGGTTGAGCGTTTGTTGAAAAAAAGTAACTGAAAAATCCGTAGAATAGCGCC |
| | ACTCTGATGGTTAATTAACCTATTCAATTAAGAATTATCTGGATGAATGTGCCATTAAATGCG |
| | CAGCATAATGGTGCGTTGTGCGGGAAAACTGCTTTTTTTTGAAAGGGTTGGTCAGTAGCGGA |
| | AAC |
| 6 | CCATCCGGTTAAGCGTATCGAAGAAGTTCTTGCCCTTGCGCTGCAGAATGAACCCTTTGGTA |
| | TGCAAGTCGTAACGGCAAAATAGTGACCTTGCGCAAAGTGCGTTAATAAAAACAAGGTTGG |
| | TGAGTGATTTCGGACTTGCCAGCCTTTTTTTGTATAGCTAATTTAGATTGCTGGTTGGGTGTG |
| | CCATCATCAACTGGTGTTGTAAGGGCATGACAGGCCTGATATAACTGCTGCGCGGTCGCGCT |
| | GTGAAGGATTCAGGTGCGATATAAATTATAAAGAGAGGAAGAGTAGAGTGAATAAATCTCA |
| | ACTGGTAGACAAGATTGCCGC |
| 7 | CGCGTCAGGTTGAACGTAAAAAAGTCGGTCTGCGCAAAGCACGTCGTCGTCCGCAGTTCTCC |
| | AAACGTTAATTGGTTTCTGCTTCGGCAGAACGATTGGCGAAAAAACCCGGTGCGAACCGGGT |
| | TTTTTTATGGATAAAGATCGTGTTATCCACAGCAATCCATTGATTATCTCTTCTTTTTCAGCAT |
| | TTCCAGAATCCCCTCACCACAAAGCCCGCAAAATCTGGTAAACTATCATCCAATTTTCTGCCC |
| | AAATGGCTGGGATTGTTCATTTTTTGTTTGCCTTACAACGAGAGTGACAGTACGCGCGGGTA |
| | GTTAACTCAACATCTGACCGGTCGAT |
| 8 | CAGAGCCGGGTTGTTGATCCGCAGGGCGTGACGGTTGCGGCAGCAGCAGAAGCGCCACAGC |
| | TGATTTTCGCAGAGGTCACGCCTGAACGCGTGGCGCAGACACGCGAGAAACTGCCGGTATTA |
| | CGCAATCGCCGTTTCGCTGTACCGCATTTATTGTGATGTTTTTTTAAACAATGCTTGATTCATC |
| | TCGTTACACATTGCTATTGTGTGCGCGCGTCGAATGACCGTTAATGAAGTCCGGTTATAATG |
| | GCGTTTTATGCAGCCTGTTTTAAGAAAGAAGGTATCTATGGGTGAGATTAGTATTACCAAAC |
| | TGCTGGTTGTGGCCGCACTGGTTGTTCTGCTGTT |
| 9 | ATATTGACACCATGACGCGCGTAATGCTGATTGGTTCTGTGACGCTGGTAATGATTGTCGAA |
| | ATTCTGAACAGTGCCATCGAAGCCGTAGTAGACCGTATTGGTGCAGAATTCCATGAACTTTC |
| | CGGGCGGGCGAAGGATATGGGGTCGGCGGCGGTGCTGATGTCCATCCTGCTGGCGATGTTTA |
| | CCTGGATCGCATTACTCTGGTCACATTTTCGATAACGCTTCCAGAATTCGATAACGCCCTGGT |
| | TTTTTGCTTAAATTTGGTTCCAAAATCGCCTTTAGCTGTATATACTCACAGCATAACTGTATA |
| | TACACCCAGGGGGCGGGATGAAAGCATTAACGGCCAGG |
| 10 | CCTGTATGAAGATGGCGTGCGCAAAGATCGCCTGGATAACAGCGATATGATTAGCCAGCTTG |
| | AAGCCCGCATTCGCGCGAAAGCGTCAATGCTGGACGAAGCGCGTCGTATCGATGTGCAACA |
| | GGTAGAAAAATAAGGTTGCTGGGAAGCGGCAGGCTTCCCGTGTATGATGAACCCGCCCGGC |
| | GCGACCCGTTGTTCGTCGCGGCCCCGAGGGTTCATTTTTTGTATTAATAAAGAGAATAAACG |
| | TGGCAAAAAATATTCAAGCCATTCGCGGCATGAACGATTATCTGCCTGGCG |
| 11 | TTCGCTAAGTCTTAGCAATAAATGAGATAAGCGGTGTGTCTTGTGGAAAAACAAGGACTAAA |
| | GCGTTACCCACTAAAAAAGATAGCGACTTTTATCACTTTTTAGCAAAGTTGCACTGGACAAA |
| | AGGTACCACAATTGGTGTACTGATACTCGACACAGCATTAGTGTCGATTTTTCATATAAAGG |
| | TAATTTTG |
| 12 | GCCCGCTGACCGACCAGAACTTCCACCTTGGACTCGGCTATACCCTTGGCGTGACGGCGCGC |
| | GATAACTGGGACTACATCCCCATTCCGGTGATCTTACCATTGGCGTCAATAGGTTACGGTCC |
| | GGCGACTTTCCAGATGACCTATATTCCCGGCACCTACAATAACGGTAACGTTTACTTCGCCTG |
| | GGCTCGTATACAGTTTTAATTCGCTAAGTCTTAGCAATAAATGAGATAAGCGGTGTGTCTTGT |
| | GGAAAAACAAGGACTAAAGCGTTACCCACTAAAAAAGATAGCGACTTTTATCACTTTTTAGC |
| | AAAGTTGCACTGGACAAAAGGTACCACAATTGGTGTACTGATACTCGACACAGCATTAGTGT |
| | CGATTTTTCATATAAAGGTAATTTTG |
| 13 | TCCCGGCTGTGCGTGAGGGAGACTGTTCTTAATCTGGCGCGCGAAGGTTGCTATTGCCCTGA |
| | AAATGGACCACCCTAGCTGAGGTCGCACAAAAAACGTGCGGCCGACTTTGGGTTACATTTCA |
| | TCCGGTCACCACCGGGTTTGCCCTTGAAACCAGAACAGGATAAAGGAGTCAGA |
| 14 | CCCGCAGCGGGTGATCCCTGGTCATTACCTCGGCACCCCGCCGGAGGGAGACAGCGCGGTG |
| | CGCTTCACAAAAACGTATCTCCAGCAGTTTGAGCAGGCGCTGAAGACGCATCAGGATTCGGC |
| | CGGGGTGATCAAGGCCATGGAGACGCAGTGGCCGGGCCTGGCGGAGTCCAGCTCGCTGGAG |
| | TTAAGCGCCAAAGTTAATACCGGCGAGATGAAGTGGTGATCCCGGCTGTGCGTGAGGGAGA |
| | CTGTTCTTAATCTGGCGCGCGAAGGTTGCTATTGCCCTGAAAATGGACCACCCTAGCTGAGG |
| | TCGCACAAAAAACGTGCGGCCGACTTTGGGTTACATTTCATCCGGTCACCACCGGGTTTGCC |
| | CTTGAAACCAGAACAGGATAAAGGAGTCAGA |
| 15 | TGAGTATTGTCAGCCGGAAGTTTGATCGGAAGGATGTGGGAACGGTATTTCGCCATGCGGTG |
| | ACCTGAAAAGTTTTGTGCCGTCTGGAGGTAAAACTGACTGCAGAAGACAGGGAGCAGTTAC |
| | TGTCCTTAATCAGTCTGGTGTATCGCGCCGGAGAGAACGCCGGTAGTGAACAACGGGCGGTT |
| | GAAATCCGGCAGGCGCTGGGTTTACAGACAGAAAACGAGTCAGGAGGTGTTTGAGGATATA |
| | TTCAGTTATCAGGCTGTTAGTCCTGGGTGGATTCGATACGACAGGGTATAATGACGTCGGCG |
| | CTTGAGGCTTTTTGCCTCATGACGTAAAGGTGGTTTGTTACCGTGTTGTGCGGCAGAAAGCA |
| | GAAAGCCCCGTAGTTAATTTTCATTAACCCACGAGGCCCCCTGTATGTCTCATCAACAACAG |
| | TATGGCCTCTTACCGTGCTCAATGCAAGGAGGAGTAAACC |
| 16 | AGTCAGGAGGTGTTTGAGGATATATTCAGTTATCAGGCTGTTAGTCCTGGGTGGATTCGATA |
| | CGACAGGGTATAATGACGTCGGCGCTTGAGGCTTTTTGCCTCATGACGTAAAGGTGGTTTGT |
| | TACCGTGTTGTGCGGCAGAAAGCAGAAAGCCCCGTAGTTAATTTTCATTAACCCACGAGGCCC |
| | CCTGTATGTCTCATCAACAACAGTATGGCCTCTTACCGTGCTCAATGCAAGGAGGAGTAAACC |
| 17 | ACGCTTCGGCCGAAAAATAAGCGCATCGGTAGCACGCTCAGTAAATCGCCGTCTATACTGAA |
| | AGAGCCTGACTGAAGGCTAATTCCAAGGAGATTGCAGG |
| 18 | GAGCGCACGCCGCCGCTGGCGAGCGCCGAGGTCACGGCGGCCTGGATGAATCAGATTATCG |
| | AACAGTGCATCCTGATGGCGCCCGAGCAATATATGTGGCTGCACCGGCGTTTTAAGACTCGC |
| | CCGGAAGGGGTACCGCCGCGTTACTGAACGCTTCGGCCGAAAAATAAGCGCATCGGTAGCA |
| | CGCTCAGTAAATCGCCGTCTATACTGAAAGAGCCTGACTGAAGGCTAATTCCAAGGAGATTG |
| | CAGG |
| 19 | CCAGCGCGCAGCGGCATGGGTCAGTAAGGGGGCTTTTGCCGCTGCACCGTAAAAAAAAGTTT |
| | GCTATCAGGTGCTGAACGTGCGTTAATGCTCGCAGGTTTGATGTACAGACCACAGAGCAGTC |
| | GAATAGAGCAGTCCTTCTAAGGTTATCCAAAGATACCCCCGTAGTGAACTTTCCCTTTATCGC |
| | TTTAAATCTGTAGTCCAGACCGCTACGCCGCAAGGCTCACTTATTTTTTTAAAGGTAATTCACT |
| 20 | AGTGGGAGTCGAGACGGGTTAGACCGTCTCCCACCGAGCTGAAATTGATGCGCCTGATTCAG |
| | GCCAATCCACAGCTTTCACGACAGTTACTCGATTAATCCAGCGCGCAGCGGCATGGGTCAGT |
| | AAGGGGGCTTTTGCCGCTGCACCGTAAAAAAAAGTTTGCTATCAGGTGCTGAACGTGCGTTA |
| | ATGCTCGCAGGTTTGATGTACAGACCACAGAGCAGTCGAATAGAGCAGTCCTTCTAAGGTTA |
| | TCCAAAGATACCCCCGTAGTGAACTTTCCCTTTATCGCTTTAAATCTGTAGTCCAGACCGCTA |
| | CGCCGCAAGGCTCACTTATTTTTTTAAAGGTAATTCACT |
| 21 | GCCGGCGATCAAAAAAGCAGCGATTTAATCGTTGCATAGGGCGCGAAATTGGCATACAATTT |
| | CGCGCCTTTTGTTTTTATGGGCCTGGCCCGTAAAACGATGTTTAATCACGGGGAGCTTCTCTG |
| | AAGCGTTAATACCCAATTTGAGGATTTAAGA |
| 22 | GCTAAAGTTCTCGGCTAATCGCTGATAACATTTGACGCAATGCGCAATAAAAGGGCATCATT |
| | TGATGCCCTTTTTGCACGCTTTCATACCAGAACCTGGCTCATCAGTGATTTTTTTTGTCATAAT |
| | CATTGCTGAGACAGGCTCTGAAGAGGGCGTTTATACACCAAACCATTCGAGCGGTAGCGCG |
| | ACGGCAAGTCAGCGTTCTCCTTTGCAATAGCAGGGAAGAGGCGCCAGAACCGCCAGCGTTG |
| | AAGCAGTTTGAACGCGTTCAGTGTATAATCCGAAACTTAATTTCGGTTTGGA |
| 23 | CTTGTGGCTGAACGACTCATCATTGTTTGTAAACAGGATGTAGCGCCAGAGTAACTGGCAAC |
| | AAAGCAGATGCTGCAGGCAGTATAAAGGCTAATGGCGTAAATCCATACTACAGAATGGTGC |
| | CAGCGGCGCGATACCCTCCAGGAATTATCTTAGAATCGAAGCGCAAATGAAACCGCGCCAA |
| | CAACGCTGACCAGTCGCGATATTGACAAAGTACAGGCGGAAGAATCGCACGAAATAACAAG |
| | ACATTGGCTGAATAAGGGCAATTGACAGGCTAATTGATTGATTAATAGTCGTTAGGGAATTT |
| | TTTGCCGTAGCACAGATAAATTAAAGTTGTGTAAAGAAGGGTAAAAAAAACCGGATGCGAG |
| | GCATCCGGTTGAAATAGGGGTAAACAGACATTCAGAACTGAATGACGGTAATAAATAAAGT |
| | TAATGATGATAGCGACTGTTATTTTAGTCACCAATGATAGTTTTGTTTTACCATTCAGTGCTA |
| | TAGAGTTATTTGTCTGTATGTGATTGATTGTGAGGAAATAAATATTTTTTTTGATTATTAGTG |
| | CGTATTTCCCAGACCATTTTGTGGTGCAAAAAGTTCCGCCATTTTTACAAATTGAAACATCTT |
| | GTGGGCATTTTGAAACATCTTAGAAGTTTTAGTATCATATTCTTGTTGGATTATTCTGCATTTT |
| | GCAGCACAATGAAATAGCCGACTGATTAGAAGGGTAATCAGTAAGCAGTGGCATAATAAAA |
| | GGCATATAACAAACAGAGGGTTAATAAC |
| 24 | TGAGGAAATAAATATTTTTTTTGATTATTAGTGCGTATTTCCCAGACCATTTTGTGGTGCAAA |
| | AAGTTCCGCCATTTTTACAAATTGAAACATCTTGTGGGCATTTTGAAACATCTTAGAAGTTTT |
| | AGTATCATATTCTTGTTGGATTATTCTGCATTTTGCAGCACAATGAAATAGCCGACTGATTAG |
| | AAGGGTAATCAGTAAGCAGTGGCATAATAAAAGGCATATAACAAACAGAGGGTTAATAAC |
| 25 | TTCTGCGAGTTTCAGAAAAAGGGGCCTGACGGCCCCTTTTTTCGACCGGGCGGCAGCAATTC |
| | ATTCAAAACTCATGTATTGTTGCTAGTAATGATCTTCATGCAGAGGTTCGCGCGGCTAATGA |
| | GAGGCTTCATCCGCAGGGGCGGGTAAAGGTTGTCATTAGTCGCGAGGATGCAGAGGATCGG |
| | GTCAATAGACGCTATATCTTTGATATGGCGTGATTTATAGATAAAAAGGATAGAATT |
| 26 | CGCCGTCCTCGCAGTACCATTGCAACCGACTTTACAGCAAGAAGTGATTCTGGCACGCATGG |
| | AACAAATTCTTGCCAGTCGGGCTTTATCCGATGACGAACGCGCACAGCTTTTATATGAGCGC |
| | GGAGTGTTGTATGATAGTCTCGGTCTGAGGGCATTAGCGCGAAATGATTTTTCACAAGCGCT |
| | GGCAATCCGACCCGATATGCCTGAAGTATTCAATTACTTAGGCATTTACTTAACGCAGGCAG |
| | GCAATTTTGATGCTGCCTATGAAGCGTTTGATTCTGTACTTGAGCTTGATC |
| 27 | AAACAAGGGTATAAGGCTATCTTGTTTGCCATTTTAGCTCCGGGGTGTGCTCGAAATGCTCA |
| | CGTACTACGTGTACGCTCCGCTTTCTGCGCGCACGCCGGAACTAAACTAGCTGCACCGATAT |
| | ACGCCTTCTATCCCTTGTTTAATGCTCAGTACCAAGATGCTGATTGCATTTTCCCCAGAAATC |
| | AGTAAAATTTTCGGGCTTTTAATATGACACCGGGCTCCGTTCCTCGATGGGGCCCGGTTGTTT |
| | TATTCACACAAGAGGATGTT |
| 28 | TCGGTTCAGGGCAATTCCATTGGTCTGATAAAGATAATATGTCCCCGTTCTCAGGGGGAAAA |
| | GATTGTCGCCGCATTCACCAAAAATGCGATATTCCGCGCAGGGCCTCCATCTTAATACGATA |
| | AAAGGCCGCTACAAGCCGTTGTTACATAACCCCTTCATTGTGGATCTCGCGGTTAATCGCCA |
| | AAAATAGCGCTAAATGACAACAAATATCATTTGCCTTCCATTCAGATAATACTTACATTCAT |
| | AACTATTAGTAATGTTTTGGCGCCAGGGCGCTTTTTATATTTCGAGGTGGAT |
| 29 | ACTATCGCGAAGACGCGCAAATCCCGGTGATGATTTTCTAAACAGCGCTTGCGTCGTGCCAG |
| | AATTTGCGTATAATGCGCGGGCCTGTCAAAGTTGACAGCCGGTTCGATATGAACCCTGATAG |
| | TGCTTTTTGCTATCAAACAATGTCCCCAATCGGGGGACTATGTAAGAACGGTTACACTCTCCC |
| | ATCAATCGTAATGGGTATGAGGAGTAATCATTTCGTCTATAAAATAATTGGAGCTCTGGACTC |
| 30 | ATATCGATCAATAAATTTGAACAATGACAGCAAATCCTTCCGCTTTTTGTTTAGCGATGTGCG |
| | GGCTACTATTTAACACATCAAGGCACGGCGCCTTATCTAAACAACTAAATGAAAGGGTTTAT |
| | ATC |
| 31 | TGAAATGGTGCAGAAGGCCGCGATGTGCGGCGTCGAGATCCTGTTCGCAGTCTCGGCGGCCA |
| | CTACCTTAGCGGTGGAAGTGGCCGAGCGCTGCAATCTGACGCTGGTGGGCTTTTGCAAGCCG |
| | GGCAGGGCGACAGTCTACACCCATCCGCAGCGTTTAATTGCGGGTTAAATATCGATCAATAA |
| | ATTTGAACAATGACAGCAAATCCTTCCGCTTTTTGTTTAGCGATGTGCGGGCTACTATTTAAC |
| | ACATCAAGGCACGGCGCCTTATCTAAACAACTAAATGAAAGGGTTTATATC |
| 32 | ATGAAATTAGGATTATTCCTGGAATTTTTTTTACCGATGGTAAAGACACAGCGTTTTTCAGGG |
| | ACTTTTTCGCGCAATGCCTGTCACACGGGGATTTCTGCCTTTTTTCTGCGTACGAAAATCAAC |
| | CATATTTGTTAAATATTGTGTACACAACCCTTTTTTTTCATATGCCTGACAGAGTTCACACTT |
| | GTAAGTTTCGAACTAAGTTGTAGACTTTACATCGCCAGGGGTGATCGGCTTACGCTGCATGT |
| | ATCAGCATAGTTAACAACAAGTCACGCCCCGGGTGAAGGATTTAACCGTGAGGTCTTTTGTA |
| | ACTTCATGGCGAATTTTGGATGATAATGAGGCGCAAAAA |
| 33 | ACCCTTTTTTTTCATATGCCTGACAGAGTTCACACTTGTAAGTTTCGAACTAAGTTGTAGACT |
| | TTACATCGCCAGGGGTGATCGGCTTACGCTGCATGTATCAGCATAGTTAACAACAAGTCACG |
| | CCCCGGGTGAAGGATTTAACCGTGAGGTCTTTTGTAACTTCATGGCGAATTTTGGATGATAA |
| | TGAGGCGCAAAAA |
| 34 | GCCGACGAAGCCTCGCCGCGCCGCTTCGTTATATACCTCAACAGGAGTACTCCGGTTGTATC |
| | GATAATGCGAGGGCTGCAGGTATTATTTCCCTGCACACAGTAAGTTAGCGGTGATGTGCCGT |
| | CTGGTTATTTTTAATGTGTGTTGTAGAATTATTCCGAATTACTGCTGAAAGACGTCGGGAAAA |
| | CGGAATAATAATTTGACTAACCAGCATTACCCGCTAGAGTTAAATATCGAACGACGAGTGAT |
| | ACGGAATATTTTCGTATCGTACTGACATAACCGATATACATGAGGTGAAAT |
| 35 | TAGAGTACGCATTCTCGATACGGATAAACGGCTCAGCGATGAGCCGTTTATTTTTTCTACCCA |
| | TATCTGGTTTGTGGTGTTATAATGCCGCGCCCTCGATATGGGGCTTTTTAACGACCCTAATTT |
| | TCGGGACTCAGTAGTAGTTGACATTAGCGGAGCACTAAA |
| 36 | ACGACCAAACTGCACGTACATGACGAGAACAACGAATGCGGTATCGGTGACGTGGTTGAAA |
| | TCCGCGAATGCCGTCCGCTGTCCAAGACTAAGTCCTGGACGCTGGTTCGCGTTGTAGAGAAA |
| | GCGGTTCTGTAATAGAGTACGCATTCTCGATACGGATAAACGGCTCAGCGATGAGCCGTTTA |
| | TTTTTTCTACCCATATCTGGTTTGTGGTGTTATAATGCCGCGCCCTCGATATGGGGCTTTTTAA |
| | CGACCCTAATTTTCGGGACTCAGTAGTAGTTGACATTAGCGGAGCACTAAA |
| 37 | GAATTTACTTACATTAAGGCGGCGAGGGGCGCCTATACTTGATAGTTCTGATACCAGAAGAA |
| | GGAAGAACT |
| 38 | ATGCCACGGCCTCCCCGGATCGGGTGGTGGAGCAGATTATGACCATGCTGTGCGGCGCGACG |
| | GCAACCCCGGTAAGTTAAGAATTTACTTACATTAAGGCGGCGAGGGGCGCCTATACTTGATA |
| | GTTCTGATACCAGAAGAAGGAAGAACT |
| 39 | TAACTATAAACGCCTATACCCTAAATAATTCGAGTGGCAGGAAGGCGGCGACGCAGCGAAT |
| | CCCCAGGAGCTTACTCAAGTAAGTGACTGGGGTGAGTGAGGAAAGCCAACACACAGGCAAC |
| | TTGAAGTATGGCGGGTATAGGTGCCGTAACCTCGGGGGAACGGCACCTTGCGTCATAAGTAC |
| | TGATAACGATAAAGTCGGGTTGAAATTGTGTATATCGGCTAAACTTAGGTTTAACAGAATGT |
| | GATGCCATGACTGCCTTATACCGCAAGGTATTTGTCATCGCTTACTTTTTGGCGTTATATGAT |
| | GGATAATGCCGGGATACGAGAGTCCCGACTCTTTTAATCTTTCAAGGAGCAAAGA |
| 40 | GCACCTTGCGTCATAAGTACTGATAACGATAAAGTCGGGTTGAAATTGTGTATATCGGCTAA |
| | ACTTAGGTTTAACAGAATGTGATGCCATGACTGCCTTATACCGCAAGGTATTTGTCATCGCTT |
| | ACTTTTTGGCGTTATATGATGGATAATGCCGGGATACGAGAGTCCCGACTCTTTTAATCTTTC |
| | AAGGAGCAAAGA |
| 41 | AGTATTAAAGGCGGAAAACGAGTTCAACCGGCGCGTCCTAATCGCATTAACAAAGAGATTC |
| | GCGCGCAAGAAGTTCGCCTCACAGGCGTCGATGGCGAGCAGATTGGTATTGTCAGTCTGAAT |
| | GAAGCTCTTGAAAAAGCTGAGGAAGCGGGCGTCGATTTAGTAGAAATCAGTCCGAATGCCG |
| | AGCCGCCAGTTTGTCGAATC |
| 42 | TGAACATCACTGATGCACAAGCTACCTATGTCGAAGAATTAACTAAAAAACTGCAAGATGC |
| | AGGCATTCGCGTTAAAGCCGACTTGAGAAATGAGAAGATTGGCTTTAAAATTCGCGAACAC |
| | ACGCTACGCCGTGTTCCTTATATGTTAGTTTGTGGCGATAAAGAGGTCGAAGCAGGCAAAGT |
| | TGCTGTTCGTACCCGCCGCGGCAAAGACTTAGGAAGCATGGATGTTAGCGAAGTCGTTGACA |
| | AACTGCTGGCGGAAATCCGCAGCAGAAGTCTTCATCAACTGGAGGAATAAAGTATTAAAGG |
| | CGGAAAACGAGTTCAACCGGCGCGTCCTAATCGCATTAACAAAGAGATTCGCGCGCAAGAA |
| | GTTCGCCTCACAGGCGTCGATGGCGAGCAGATTGGTATTGTCAGTCTGAATGAAGCTCTTGA |
| | AAAAGCTGAGGAAGCGGGCGTCGATTTAGTAGAAATCAGTCCGAATGCCGAGCCGCCAGTT |
| | TGTCGAATC |
| 43 | TCTGGCCTTAATCTGGTGCTGAAGAATATTCAGTGCCGGTTTTGGCTATAGTTTTTTTTAACC |
| | TCGCCGCAAGGATCTGTAGCGGGGCATTTGAAACAACCCCATCCAGCAGGACGCCAG |
| 44 | TGCACCGGTGAAGATATTTCTGGATGCCAGTTCGGAAGAACGTGCAAACAGAAGAATGCTA |
| | CAGTTGCAGGAAAAAGGCTTTAGTGTTAACTTTGAACGGCTTTTAGCCGAGATCAAAGAACG |
| | CGATGACCGTGATCGTAACAGGCCTATCGCGCCTTTAGTGGCTGCTTCCGATGCACTGTTGCT |
| | GGATTCAACCAGTATGTCTATCGACGAAGTCATCGAAAAAGCACTGGCTTATGCCACAGAAA |
| | TTCTAGGATTACCGCAAAAACAAACCCGGTAATCTGGCCTTAATCTGGTGCTGAAGAATATT |
| | CAGTGCCGGTTTTGGCTATAGTTTTTTTTAACCTCGCCGCAAGGATCTGTAGCGGGGCATTTG |
| | AAACAACCCCATCCAGCAGGACGCCAG |
| 45 | TACAGTAGCGCCTCTCAAAAATAGATAAACGGCTCATGTACGTGGGCCGTTTATTTTTTCTAC |
| | CCATAATCGGGAACCGGTGTTATAATGCCGCGCCCTCATATTGTGGGGATTTCTTAATGACCT |
| | ATCCTGGGTCCTAAAGTTGTAGTTGACATTAGCGGAGCACTAAC |
| 46 | TCTGTAACAGAAGTTTTACAGCTCCTTTCCATCTGGAAAGGAGCTGTTCGTCTCACGGACGC |
| | AGGACGCGTTTGTGTTAAGCAAGCGGATGACAGGATGTTCATCCAATGTTTGTCTCCGGGAG |
| | TAGAA |
| 47 | TCAAGCGAGTTTCAGTGTAAAGGGGCCAATAGGCCCCTTTATTCTAGGAAGCGCAGCCAAAT |
| | CAGGGTACTGTATGGCTGCGGTTTCTACTGTTATTCTAAGAACATGAACTTCCGTTACAGATG |
| | TTTTCGCGCGGCTAATGAGAGACTTTATTACCACATTGCCAGGTATATAAGGATTGTCATTAG |
| | TCGCGAGAATGTAGTGAGAAGCTCGGATATTTATCGGCGTGAACTGCTGTCATAACAGCTGC |
| | GCGTCATACAAAAGGATATTACA |
| 48 | AAATTACGAAATTATTTGCGTTTTTTGCGGTAAAAACCGCAAAATAGAGCAAATTCGTGGTT |
| | TGACCAGCCTGGATTTAGTTGCATCTTTTTCAACATTTTATACACTACGAAAACCATCGCGAA |
| | AGCGAGTTTTGATAGGAAATTTAAGAGT |
| 49 | GAATATTTAGGCGAAAATGGCAAGGGTATCATGCTCAATGTGGTTGATTCTGCATCTATTGA |
| | GCAAGTATTGGCGACGATTCGAGCTGAATTTGGCGAAATTGATATTTTAGTTAATAATGCCG |
| | GCATCACCCGTGATAACCTTCTCATGCGTATGAAGGATGATGAGTGGCAGGATATCCTGGAT |
| | ACGAACCTGACTTCAGTGTTTCGGCTGTCAAAAGCTGTCATGCGAGCTATGATGAAGAAACG |
| | GTGTGGACGGATTATTACAATTGGTTCCGTTGTTGGCACCATGGGTAACGCAGGGCAGGCGA |
| | ACTACGCGGCGGCTAAAGCTGGCTTGATTGGTTTTAGTAAGTCTTTGGCACGTGAGGTCGCT |
| | TCACGTGGCATTACTGTCAACGTCGTGGCTCCCGGCTTTATTGAGACGGATATGACAAGGGC |
| | GTTGACAGATGATCAACGCGCAGGCATTTTGTCATCAGTTCCAGCCAACCGGTTGGGCGATG |
| | CCAAAGAAATTGCCAGCGCCGTTGCTTTTTTAGCCTCTGACGAGGCCAGCTACATCACGGGT |
| | GAAACATTACATGTCAATGGCGGCATGTATATGATTTAAAAATTACGAAATTATTTGCGTTTT |
| | TTGCGGTAAAAACCGCAAAATAGAGCAAATTCGTGGTTTGACCAGCCTGGATTTAGTTGCAT |
| | CTTTTTCAACATTTTATACACTACGAAAACCATCGCGAAAGCGAGTTTTGATAGGAAATTTA |
| | AGAGT |
| 50 | AATTTTTTTTCACAAAGCGTAGCGTTATTGAATCGCACATTTTAAACTGTTGGCCGCTGTGGA |
| | AGCGAATATTGGTGAAAGGTGCGGTTTTAAGGCCTTTTTCTTTGACTCTCTGTCGTTACAAAG |
| | TTAATATGCGCGCCCT |
| 51 | TTAAAAACGTGACCACGAGCATTAATAAACGCCACGAAATGTGGCGTTTATTTATTCAAAAA |
| | GTATCTTCTTTCATAAAAAGTGCTAAATGCAGTAGCAGCAAAATTGGGATAAGTCCCATGGA |
| | ATACGGCTGTTTTCGCTGCAATTTTTAACTTTTTCGTAAAAAAAGATGTTTCTTTGAGCGAAC |
| | GATCAAAATATAGCGTTAACCGGCAAAAAATTATTCTCATTAGAAAATAGTTTGTGTAATAC |
| | TTGTAACGCTACATGGAGATTAACTTAATCTAGAGGGTTTTATA |
| 52 | ACACCCTCCTTCCATCACCATTGTGATTATGGTTATTAAATTTTTATAGAAATAACTTAACGA |
| | TCATTATTAAAAATAGTTGCGCACAAGTCCAGCGGAGTTTATTATTTAATTATCGAGCGATA |
| | AGAAAATCGCTCAAACCCGCAAAACTGCGCAGCTAAAAACGCTTTTTAAGCATACTATCCAG |
| | GACGTAACATC |
| 53 | TAAAAAATTCCTGAACGGGCGGTAAATGAAAAAGGTTTTATCAATCATTCATGCTGTGAGCA |
| | CGGTTTGCAAGGCTTGCAGTATGAATTGATGCAACAATGTGTGGTGACCAGAAATCACTGCC |
| | GGTTCATTCAGATAGGTCAAAGGTATCGGACTGACAGGTAATTCCTGCTTTTTTTTATGATCT |
| | GCAACAGGCATAGTATCGACATCAAAAAGGTGATGTGGATAACAAAAAACAAACATTCCCT |
| | TTTCATTTATCTCGTTGGCATTAACAAAGGAGTCTCC |
| 54 | TCAATCTCATCAGTTCTGTGAACCGTCCCGCAATTCCCTGCAATACAAGAGGTTGTTGTTAAA |
| | GAACTCTGAGACTTACGTCAAAGACTGATAGCCGGATACTATCTGATTGATTGGTGCGATGG |
| | GGTTTATTCACCCGCAGCTTGCCCCTATACTGACAGTCGTTTTGTTCATCCTTTCCTTTCACCT |
| | ACGACGCCCTCTTGGGTTTCATAAGGAGTAATATT |
| 55 | TACCGGCGGTTTGCAACCAGGTAAAGACATCCGGCCACTGCCAGCTTGATTCATCGATAATC |
| | ACCTGCGTGTTGTCCGGCAATACGCGGGGGATATTTTCCCAGAAGCCGCCACCGGTCAGATG |
| | GACGATGCCGTGAACATCCACGTTTTCGATCAGGTTCAGGATCGATTTCACGTAAATTTTGGT |
| | CGGTGCGAGCAAATGATCAGCCAGCGGTTTGCCTGCCAGATCGGTGGTTTCCGGGTCGGTCT |
| | TGCTGACTTCCAGAATTTTGCGCACCAGAGAATAACCGTTAGAATGCGGGCCACTGGCGGCC |
| | AGACCAATCAGCACATCCCCGTCAGCCACTTTGCTGCCGTCGATGATTTCTGATTTTTCCACC |
| | ACGCCCACGCAGAAGCCTGCCACGTCGTAATCTTCGCCGTGATACATGCCCGGCATTTCAGC |
| | GGTTTCACCGCCAACTAACGCACAGCCAGACTGTTTACAGCCTTCTGCGATACCCGTGATCA |
| | CGCTGGCAGCCGTATCGACGTCCAGTTTGCCGGTAGCGTAATAATCGAGGAAGAACAGGGG |
| | TTCGGCGCCCTGAACGATCAAATCGTTGACGCACATCGCGACCAGGTCGATACCGATAGTAT |
| | CGTGGCGTTTCAAATCCATCGCCAGACGCAGCTTGGTGCCAACGCCGTCGGTACCCGATACC |
| | AGCACGGGTTCACGATATTTTTGCGGCAGCGCGCAGAGGGCACCAAAACCGCCCAGTCCAC |
| | CCATGACTTCAGGGCGGCGAGTCTGTTTTACTACACCTTTAATGCGGTCTACCAATGCGTTAC |
| | CGGCATCGATATCTACGCCTGCGTCTTTATAGCTGAGAGAGGTTTTGTCGGTCACTGCGAAG |
| | TCCCCACGGCGGTTTGGGTTGGTGGTTGAAGAATAAAGCGGGGCAATTCTAACAGTGCAAGC |
| | AAACGTTTGCGAGCGCCTTATTCAGAGTCACTATCTATACTTAAAAATACAACACTTAGCCG |
| | AAGTCATTGGAGTTGCAGCAAGGCAGCAAACGAGCGAATCCCGATGAGCTGACTTGAGTCA |
| | GTGATTCGGGTGAGAGAGAGCAGCTAACGCAGCTGCGGCTTCAATGAAGCAGGGTAAGTTG |
| | ATCCAGATCAGGCTATTTGGTATGGCGTTCAAAAAAAATGGCGTTATAATCTCGCGATTTTTT |
| | TTTGCAGCTCAACCACCTTAGGAGAATAAATAATGAAGATCGTCGAGGTGAAACACCCGCTG |
| | GTGAAACACAAGCTGG |
| 56 | CGGCATCGATATCTACGCCTGCGTCTTTATAGCTGAGAGAGGTTTTGTCGGTCACTGCGAAG |
| | TCCCCACGGCGGTTTGGGTTGGTGGTTGAAGAATAAAGCGGGGCAATTCTAACAGTGCAAGC |
| | AAACGTTTGCGAGCGCCTTATTCAGAGTCACTATCTATACTTAAAAATACAACACTTAGCCG |
| | AAGTCATTGGAGTTGCAGCAAGGCAGCAAACGAGCGAATCCCGATGAGCTGACTTGAGTCA |
| | GTGATTCGGGTGAGAGAGAGCAGCTAACGCAGCTGCGGCTTCAATGAAGCAGGGTAAGTTG |
| | ATCCAGATCAGGCTATTTGGTATGGCGTTCAAAAAAAATGGCGTTATAATCTCGCGATTTTTT |
| | TTTGCAGCTCAACCACCTTAGGAGAATAAATAATGAAGATCGTCGAGGTGAAACACCCGCTG |
| | GTGAAACACAAGCTGG |
| 57 | CGCGGTTTGGTTGGTCAAATTTCACACAAAATCACGTTGATCGACTATACTGGTTTCGTCGCG |
| | CTGACTGAGAAACATGCCCAGCAAACAGCATGGTGAAACATCGATGTGCTGTATATTTCTTG |
| | ACACCCTCTTAGGTCAGCCCTAAAATTCTGCGTCCCCATATTAGCTAATGCTTTTTATGGGGC |
| | GATTTATCACGCGTTTACAAAGTAGTTTATGAACCAAAATCCAGGAGCTTTTTAATGGCAAC |
| | AATTAATCAGCTGGTACGCAAACCACGCTCTACGAAGGTTGCTAAAAGCAACGTTCCAGCGC |
| 58 | CCTGGTTGAGTCTGCTCCAGCAGCTCTGAAAGAAGGCATCAGCAAAGATGACGCTGAAGCTC |
| | TGAAAAAATCTCTGGAAGAAGCTGGTGCTTCTGTTGAAGTTAAGTAAGTTTAACTTCCCGGA |
| | GTGCAGTCTGTCCTAACAGGCTGATGGCTGGTGACTTTTTAGTCACCAGCCTTTTTGCGCTAT |
| | AGAGTGTCAGTGATGTTTCACACTGTTTGAGCACTGAACTACTCTAATATCTCTTTCTATAGA |
| | CGCCTTAATATATTGTTGCCTCTTGCTGTAGCTCATCTACAGATAACGCACAACGAAATGATT |
| | TAAGAGTGGTAGAAAACAGATATTGCGGAAAGCGTTTCTGCTTTCCGGTCGACATAAACGGT |
| | GTTGCATGAACTGTCCTTCTCAGGGCAGACAAGATTGGGTCACTGATCAGCGAGCTGAGGAA |
| | CCCTATGGTTTACTCCTATACCGAGAAAAAACGCATTCGTAAGGATTTTGGT |
| 59 | TGAATATCACTGACTCACAAGCTACCTATGTCGAAGAATTAACTAAAAAACTGCAAGATGCA |
| | GGCATTCGCGTTAAAGCCGACTTGAGAAATGAGAAGATTGGCTTTAAAATTCGCGAACACAC |
| | GCTACGCCGTGTTCCTTATATGTTAGTTTGTGGCGATAAAGAGGTCGAAGCAGGCAAAGTTG |
| | CTGTTCGTACTCGTCGCGGCAAAGACTTAGGAAGCATGGATGTTAGCGAAGTCGTTACAAAC |
| | TGCGGCGGAAATCCGCAGCAGAAGTCTTCATCAACTGGAGGAATAAAGTATTAAAGGCGGA |
| | AAACGAGTTCAACCGGCGCGTCCTAATCGCATTAACAAAGAGATTCGCGCGCAAGAAGTTC |
| | GCCTCACCGGCGTCGATGGCGAGCAGATTGGTATTGTCAGTCTGAATGAAGCTCTTGAAAAA |
| | GCTGAGGAAGCGGGCGTCGATTTAGTAGAAATCAGTCCGAATGCCGAGCCGCCAGTTTGTCG |
| | AATC |
| 60 | CTGGGGTCACTGGAGCGCTTTATCGGCATCCTGACCGAAGAATTTGCCGGTTTCTTCCCGACC |
| | TGGCTGGCCCCTGTTCAGGTTGTGGTGATGAATATCACTGATTCTCAAGCTGAATATGTCAAC |
| | GAATTGACCCGTAAATTGCAAAATGCGGGCATTCGTGTAAAAGCGGACTTGAGAAACGAGA |
| | AGATTGGCTTTAAAATCCGCGAGCACACTTTACGTCGTGTCCCTTATATGTTGGTCTGTGGTG |
| | ATAAAGAGGTGGAAGCAGGCAAAGTGGCCGTTCGCACCCGCCGCGGTAAAGACCTGGGCAG |
| | CCTGGACGTAAGTGAAGTGATTGAGAAGCTGCAACAAGAGATTCGCAGCCGCAGTCTTCAA |
| | CAACTGGAGGAATAAGGTATTAAAGGCGGAAAACGAGTTCAAACGGCACGTCCGAATCGTA |
| | TCAATGGCGAGATTCGCGCCCAGGAAGTTCGCTTAACTGGTCTGGAAGGTGAGCAGCTGGGT |
| | ATT |
| 61 | TTCTTGGTTCTCTGGAGCGCTTTATCGGCATCCTGACTGAAGAATTTGCAGGCTTCTTCCCAA |
| | CCTGGCTTGCACCCGTGCAGGTAGTTGTGATGAACATCACTGATTCGCAGGCTGAATACGTT |
| | AACGAATTGACCCGTAAACTGCAAAATGCGGGCATTCGTGTAAAAGCAGACTTGAGAAACG |
| | AGAAGATTGGCTTTAAAATCCGCGAGCACACTTTACGTCGTGTCCCTTATATGCTGGTTTGTG |
| | GTGACAAAGAGGTCGAAGCCGGCAAAGTTGCTGTGCGTACCCGTCGCGGTAAAGACCTGGG |
| | TAGCCTGGACGTAAATGATGTTATCGAGAAGCTGCAACAAGAGATTCGCAGCCGCAGTCTTC |
| | AACAACTGGAGGAATAAGGTATTAAAGGCGGAAAACGAGTTCAAACGGCGCGTCCCAATCG |
| | TATTAATGGCGAGATTCGCGCCACGGAAGTTCGCTTAACAGGTCTGGAAGGCGAGCAGCTTG |
| | GTATT |
| 62 | TGAATATTACCGATTCTCAGGCGGATTACGTTAAAGAATTGACGCAGAAACTTCAAAATGCG |
| | GGCATTCGCGTAAAAGCAGACTTGAGAAATGAGAAGATTGGCTTTAAAATCCGCGAGCACA |
| | CTTTACGTCGTGTCCCGTATATGTTGGTCTGTGGTGATAAAGAGGTGGAAGCAGGCAAAGTT |
| | GCCGTTCGCACCCGCCGTGGTAAAGACCTGGGCAGCCTGGACGTAAGTGAAGTGATTGAGA |
| | AGCTGCAACAAGAGATTCGCAGCCGCAGTCTTCAACAACTGGAGGAATAAGGTATTAAAGG |
| | CGGAAAACGAGTTCAAACGGCACGTCCGAATCGTATCAATGGCGAGATTCGCGCCCAGGAA |
| | GTTCGCTTAACAGATCTTGAAGGTGAACCACTGGGGATTGTGAGTCTGAGAGAAGCGATCGA |
| | AAAAGCTGAAGAAGCTGGAGTAGATTTAGTTGAAATCAGCCCTAACGCCGAACCGCCAGTT |
| | TGTCGTATT |
| 63 | TGAATATCACTGACTCACAAGCTACCTATGTCGAAGAATTAACTAAAAAACTGCAAGATGCA |
| | GGCATTCGCGTTAAAGCCGACTTGAGAAATGAGAAGATTGGCTTTAAAATTCGCGAACACAC |
| | GCTACGCCGTGTTCCTTATATGTTAGTTTGTGGCGATAAAGAGGTCGAAGCAGGCAAAGTTG |
| | CTGTTCGTACTCGTCGCGGCAAAGACTTAGGAAGCATGGATGTTAGCGAAGTCGTTGACAAA |
| | CTGCTGGCGGAAATCCGCAGCAGAAGTCTTCATCAACTGGAGGAATAAAGTATTAAAGGCG |
| | GAAAACGAGTTCAACCGGCGCGTCCTAATCGCATTAACAAAGAGATTCGCGCGCAAGAAGT |
| | TCGCCTCACCGGCGTCGATGGCGAGCAGATTGGTATTGTCAGTCTGAATGAAGCTCTTGAAA |
| | AAGCTGAGGAAGCGGGCGTCGATTTAGTAGAAATCAGTCCGAATGCCGAGCCGCCAGTTTG |
| | TCGAATC |
| 64 | TACAGTAGCGCCTCTCAAAAATAGATAAACGGCTCATGTACGTGGGCCGTTTATTTTTTCTAC |
| | CCATAATCGGGAACCGGTGTTATAATGCCGCGCCCTCATATTGTGGGGATTTCTTAACGACC |
| | TATCCTGGGTCCTAAAGTTGTAGTTGACATTAGCGGAGCACTAAC |
| 65 | AATTTTTTTTCACAAAGCGTAGCGTTATTGAATCGCACATTTTAAACTGTTGGCCGCTGTGGA |
| | AGCGAATATTGGTGAAAGGTGCGGTTTTAAGGCCTTTTTCTTTGACTCTCTGTCGTTACAAAG |
| | TTAATATGCGCGCCCT |
| 66 | TTAAAAACGTGACCACGAGCATTAATGAACGCTGCGAAATGTGGCGTTTATTTATTCAAAAA |
| | GTATCTTCTTTCATAAAAAGTGCTAAATGCAGTAGCCGCAAAATTGGGATAAGTCCCATGGA |
| | ATACGGCTGTTTTCGCTGCAATTTTTAACTTTTTCGTAAAAAAAGATGCTTCTTTGAGCGAAC |
| | GATCAAAATATAGCGCTTACCGACAAAAAATTATTCTCATTAGAAAATAGTTTGTGTAATAC |
| | TTGTAACGCTACATGGAGATTAACTTAATCTAGAGGGTTTTATA |
| 67 | AGCGTCAGGTACCGGTCATGATTCACCGTGCGATTCTCGGTTCCCTGGAGCGCTTCATTGGC |
| | ATCCTGACCGAAGAGTTCGCTGGCTTCTTCCCAACCTGGATTGCACCAGTGCAGGTAGTGGT |
| | CATGAATATTACCGATTCTCAGGCTGAATACGTTAACGAATTGACGCGTAAACTACAAAATG |
| | CGGGCATTCGTGTAAAAGCAGACTTGAGAAATGAGAAGATTGGCTTTAAAATCCGCGAGCA |
| | CACTTTACGTCGTGTCCCGTATATGTTGGTCTGTGGCGACAAAGAAGTCGAAGCCGGCAAAG |
| | TGGCCGTGCGCACCCGTCGCGGGAAAGACCTCGGCAGCATGGACGTAAGTGAAGTGATTGA |
| | GAAGCTGCAACAAGAGATTCGCAGCCGCAGTCTTCAACAACTGGAGGAATAAGGTATTAAA |
| | GGCGGAAAACGAGTTCAAACGGCACGTCCGAATCGTATCAATGGCGAGATTCGCGCCCTGG |
| | AAGTTCGC |
| 68 | GCCCGCTGACCGACCAGAACTTCCACCTTGGACTCGGCTATACCCTTGGCGTGACGGCGCGC |
| | GATAACTGGGACTACATCCCCATTCCGGTGATCTTACCATTGGCGTCAATAGGTTACGGTCC |
| | GGCGACTTTCCAGATGACCTATATTCCCGGCACCTACAATAACGGTAACGTTTACTTCGCCTG |
| | GGCTCGTATACAGTTTTAATTCGCTAAGTCTTAGCAATAAATGAGATAAGCGGTGTGTCTTGT |
| | GGAAAAACAAGGACTAAAGCGTTACCCACTAAAAAAGATAGCGACTTTTATCACTTTTTAGC |
| | AAAGTTGCACTGGACAAAAGGTACCACAATTGGTGTACTGATACTCGACACAGCATTAGTGT |
| | CGATTTTTCATATAAAGGTAATTTTG |
| 69 | GCTAAAGTTCTCGGCTAATCGCTGATAACATTTGACGCAATGCGCAATAAAAGGGCATCATT |
| | TGATGCCCTTTTTGCACGCTTTCATACCAGAACCTGGCTCATCAGTGATTTTTTTTGTCATAAT |
| | CATTGCTGAGACAGGCTCTGAAGAGGGCGTTTATACACCAAACCATTCGAGCGGTAGCGCG |
| | ACGGCAAGTCAGCGTTCTCCTTTGCAATAGCAGGGAAGAGGCGCCAGAACCGCCAGCGTTG |
| | AAGCAGTTTGAACGCGTTCAGTGTATAATCCGAAACTTAATTTCGGTTTGGA |
| 70 | TCGCCACGGCGATAACCATAGGTTTTCGGCGTGGCCACATCCATGGTAAATCCCANTTTTTCC |
| | AGCACGCGCGCCACTTCATCGGGTCTTAAATACATAGATTTTCCTCGTCATCTTTCCAAAGCC |
| | TCGCCACCTTACATGACTGAGCATGGACCGTGACTCAGAAAATTCCACAAACGAACCTGAAA |
| | GGCGTGATTGCCGTCTGGCCTTAAAAATTATGGTCTAAACTAAAATTCACATCGAAAACGAG |
| | GGAGGATCCTATGTTTAACAGACCGAATCGCCGTGACGTAGATGAAGGTGTTGAGGATATTA |
| | ACCACGATGTTAACCAGCTCG |
| 71 | CGCGTCAGGTTGAACGTAAAAAAGTCGGTCTGCGCAAAGCACGTCGTCGTCCGCAGTTCTCC |
| | AAACGTTAATTGGTTTCTGCTTCGGCAGAACGATTGGCGAAAAAACCCGGTGCGAACCGGGT |
| | TTTTTTATGGATAAAGATCGTGTTATCCACAGCAATCCATTGATTATCTCTTCTTTTTCAGCAT |
| | TTCCAGAATCCCCTCACCACAAAGCCCGCAAAATCTGGTAAACTATCATCCAATTTTCTGCCC |
| | AAATGGCTGGGATTGTTCATTTTTTGTTTGCCTTACAACGAGAGTGACAGTACGCGCGGGTA |
| | GTTAACTCAACATCTGACCGGTCGAT |
| 72 | ATATTGACACAATGACGCGCGTACTGCTGATTGGTTCTGTGACGCTGGTGATGATTGTCGAA |
| | ATTCTGAACAGCGCCATCGAAGCTGTGGTAGACCGTATTGGTGCGGAATTCCATGAACTTTC |
| | CGGGCGGGCGAAGGATATGGGGTCGGCGGCGGTGCTGATGTCCATCCTGCTGGCGCTGTTTA |
| | CCTGGATCGCATTACTCTGGTCACATTTTGGATAACGCTTCCAGAATTCGATAACGCCCTGGT |
| | TTTTTGCTTAAATTTGGTTCCAAAATCGCCTTTAGCTGTATATACTCACAGCATAACTGTATA |
| | TACACCCAGGGGGCGGGATGAAAGCATTAACGGCCAGG |

TABLE 9
Promoter activity in vitro and in planta

| | | | | in vitro | in planta |
|---|---|---|---|---|---|
| | | Native gene | Native gene | transcription | transcription |
| | Species | name | function | observed | observed |
| 1 | Kosakonia sacchari | lpp | cell surface | Yes | Yes |
| | | | lipoprotein | | |
| 2 | Kosakonia sacchari | hypothetical | unknown | Yes | NA |
| | | protein | | | |
| 3 | Kosakonia sacchari | mntP 1 | putative manganese | No | NA |
| | | | efflux pump | | |
| 4 | Kosakonia sacchari | acpP | acyl carrier | No | NA |
| | | | protein | | |
| 5 | Kosakonia sacchari | ompX | outer membraine | Yes | Yes |
| | | | protein X precursor | | |
| 6 | Kosakonia sacchari | hupB | DNA-binding | NA | NA |
| | | | protein HU-beta | | |
| 7 | Kosakonia sacchari | sspA | Stringent | Yes | NA |
| | | | starvation | | |
| | | | protein A | | |
| 8 | Kosakonia sacchari | tatE | Sec-independent | NA | NA |
| | | | protein translocase | | |
| | | | protein TatE | | |
| 9 | Kosakonia sacchari | lexA 3 | LexA repressor | Yes | NA |
| 10 | Kosakonia sacchari | hisS | Histidine--tRNA | Yes | NA |
| | | | ligase | | |
| 11 | Klebsiella variicola | | | Yes | NA |
| 12 | Klebsiella variicola | | | Yes | NA |
| 13 | Klebsiella variicola | | | No | NA |
| 14 | Klebsiella variicola | | | No | NA |
| 15 | Klebsiella variicola | | | No | NA |
| 16 | Klebsiella variicola | | | Yes | NA |
| 17 | Klebsiella variicola | | | Yes | NA |
| 18 | Klebsiella variicola | | | Yes | NA |
| 19 | Klebsiella variicola | | | Yes | NA |
| 20 | Klebsiella variicola | | | Yes | NA |
| 21 | Klebsiella variicola | | | No | NA |
| 22 | Klebsiella variicola | | | Yes | NA |
| 23 | Klebsiella variicola | | | NA | NA |
| 24 | Klebsiella variicola | | | Yes | NA |
| 25 | Klebsiella variicola | | | NA | NA |
| 26 | Klebsiella variicola | | | Yes | NA |
| 27 | Klebsiella variicola | | | No | NA |
| 28 | Klebsiella variicola | | | Yes | NA |
| 29 | Klebsiella variicola | | | No | NA |
| 30 | Klebsiella variicola | | | No | NA |
| 31 | Klebsiella variicola | | | Yes | NA |
| 32 | Klebsiella variicola | | | Yes | NA |
| 33 | Klebsiella variicola | | | Yes | NA |
| 34 | Klebsiella variicola | | | Yes | NA |
| 35 | Klebsiella variicola | | | Yes | NA |
| 36 | Klebsiella variicola | | | Yes | NA |
| 37 | Klebsiella variicola | | | Yes | NA |
| 38 | Klebsiella variicola | | | Yes | NA |
| 39 | Klebsiella variicola | | | Yes | NA |
| 40 | Klebsiella variicola | | | Yes | NA |
| 41 | Rahnella aquatilis | infC | | NA | NA |
| 42 | Rahnella aquatilis | infC | | NA | NA |
| 43 | Rahnella aquatilis | rpsA | | NA | NA |
| 44 | Rahnella aquatilis | rpsA | | NA | NA |
| 45 | Rahnella aquatilis | rplN | | NA | NA |
| 46 | Rahnella aquatilis | nlpI | | NA | NA |
| 47 | Rahnella aquatilis | pnp | | NA | NA |
| 48 | Rahnella aquatilis | acpP 1 | | NA | NA |
| 49 | Rahnella aquatilis | acpP 1 | | NA | NA |
| 50 | Rahnella aquatilis | hypothetical | unknown | NA | NA |
| | | protein | | | |
| 51 | Rahnella aquatilis | lpp | cell surface | NA | NA |
| | | | lipoprotein | | |
| 52 | Rahnella aquatilis | hypothetical | unknown | NA | NA |
| | | protein | | | |
| 53 | Rahnella aquatilis | grxA | | NA | NA |
| 54 | Rahnella aquatilis | tsaA | | NA | NA |
| 55 | Rahnella aquatilis | upp | | NA | NA |
| 56 | Rahnella aquatilis | upp | | NA | NA |
| 57 | Rahnella aquatilis | rpsL | | NA | NA |
| 58 | Rahnella aquatilis | rpoB | | NA | NA |
| 59 | Rahnella aquatilis | infC | | NA | NA |
| 60 | Kluyvera intermedia | infC | | NA | NA |
| 61 | Kosakonia pseudosacchari | infC | | NA | NA |
| 62 | Enterobacter sp. | infC | | NA | NA |
| 63 | Rahnella aquatilis | infC | | NA | NA |
| 64 | Rahnella aquatilis | rplN | | NA | NA |
| 65 | Rahnella aquatilis | hypothetical | unknown | NA | NA |
| | | protein | | | |
| 66 | Rahnella aquatilis | lpp | cell surface | NA | NA |
| | | | lipoprotein | | |
| 67 | Klebsiella sp. | infC | | NA | NA |
| 68 | Klebsiella sp. | | | NA | NA |
| 69 | Klebsiella sp. | | | NA | NA |
| 70 | Kosakonia pseudosacchari | hypothetical | unknown | NA | NA |
| | | protein | | | |
| 71 | Kosakonia pseudosacchari | sspA | Stringent | NA | NA |
| | | | starvation | | |
| | | | protein A | | |
| 72 | Kosakonia pseudosacchari | lexA 3 | LexA repressor | NA | NA |

TABLE 10
Species origin of promoter sequences disclosed herein

| SEQ ID NO: | Species |
|---|---|
| 1-10 | Kosakonia sacchari |
| 11-40 | Klebsiella variicola |
| 41-59, 63-66 | Rahnella aquatilis |
| 60 | Kluyvera intermedia |
| 61, 70-72 | Kosakonia pseudosacchari |
| 62 | Enterobacter sp. |
| 67-69 | Klebsiella sp. |

What is claimed is:

1. A method of increasing an amount of atmosphere derived nitrogen in a plant, said method comprising contacting said plant with a genetically engineered bacterium, wherein said genetically engineered bacterium comprises (a) a nitrogen fixation or nitrogen assimilation coding sequence, operably linked to (b) an inserted infC promoter, wherein the inserted infC promoter comprises a nucleotide sequence having at least 90% sequence identity to the sequence set forth in SEQ ID NO: 67, 41, 42, 59, 60, 61, 62, or 63.

2. The method of claim 1, wherein the inserted infC promoter comprises a nucleotide sequence having at least 95% sequence identity to the sequence set forth in SEQ ID NO: 67, 41, 42, 59, 60, 61, 62, or 63.

3. The method of claim 1, wherein said nitrogen fixation or nitrogen assimilation coding sequence is selected from the group consisting of nifA, nifL, ntrB, ntrC, polynucleotide encoding glutamine synthetase, glnA, glnB, glnK, draT, amtB, polynucleotide encoding glutaminase, glnD, glnE, nifJ, nifH, nifD, nifK, nifY, nifE, nifN, nifU, nifS, nifV, nifW, nifZ, nifM, nifF, nifB, nifQ, and a gene associated with biosynthesis of a nitrogenase enzyme.

4. The method of claim 1, wherein said engineered bacterium is a genetically engineered diazotrophic bacterium.

5. The method of claim 1, wherein said genetically engineered bacterium is non-intergeneric.

6. The method of claim 1, wherein said inserted infC promoter is from the same species as the bacterium.

7. The method of claim 1, wherein said inserted infC promoter is from the same strain as the bacterium.

8. The method of claim 1, wherein said genetically engineered bacterium fixes atmospheric nitrogen under non nitrogen limiting conditions.

9. The method of claim 1, wherein said genetically engineered bacterium fixes more atmospheric nitrogen than a non-engineered bacterium of the same species.

10. The method of claim 1, wherein said inserted infC promoter is a native sequence inserted in a non-native context.

11. The method of claim 1, wherein the method decreases the amount of nitrogen fertilizer required between planting and harvesting of a crop of said plant.

12. The method of claim 1, wherein said genetically engineered bacterium is of the genus Klebsiella, Kosakonia, Rahnella, Enterobacter, or Kluyvera.

13. The method of claim 12, wherein said genetically engineered bacterium is of the genus Klebsiella.

14. The method of claim 12, wherein said genetically engineered bacterium is of the genus Klebsiella, and wherein the inserted infC promoter comprises a nucleotide sequence having at least 90% sequence identity to the sequence set forth in SEQ ID NO: 67.

15. The method of claim 14, wherein said genetically engineered bacterium is Klebsiella variicola.

16. The method of claim 12, wherein said genetically engineered bacterium is of the genus Kosakonia, and wherein the inserted infC promoter comprises a nucleotide sequence having at least 90% sequence identity to the sequence set forth in SEQ ID NO: 61.

17. The method of claim 12, wherein said genetically engineered bacterium is of the genus Rahnella, and wherein the inserted infC promoter comprises a nucleotide sequence having at least 90% sequence identity to the sequence set forth in SEQ ID NO: 41, 42, 59, or 63.

18. The method of claim 12, wherein said genetically engineered bacterium is of the genus Enterobacter, and wherein the inserted infC promoter comprises a nucleotide sequence having at least 90% sequence identity to the sequence set forth in SEQ ID NO: 62.

19. The method of claim 12, wherein said genetically engineered bacterium is of the genus Kluyvera, and wherein the inserted infC promoter comprises a nucleotide sequence having at least 90% sequence identity to the sequence set forth in SEQ ID NO: 60.

20. The method of claim 1, wherein a plant contacted with the genetically engineered bacterium exhibits an increase in the amount of atmosphere derived nitrogen present in the plant, in comparison to a control plant contacted with a bacterium of the same strain but without the inserted infC promoter.

21. A composition for increasing the amount of atmosphere derived nitrogen in a plant, said composition comprising:
a) Klebsiella variicola having an inserted promoter sequence that is at least 95% identical to SEQ ID NO: 67, operably linked to a nitrogen fixation or nitrogen assimilation coding sequence, and
b) a plant or a seed.

22. The composition of claim 21, wherein the plant or seed is selected from corn and wheat.
